(19) United States
(12) Patent Application Publication
(10) Pub. No.: US 2023/0340078 A1
(43) Pub. Date: Oct. 26, 2023

(54) TREATMENT OF HEREDITARY ANGIOEDEMA WITH LIVER-SPECIFIC GENE THERAPY VECTORS

(71) Applicant: BIOMARIN PHARMACEUTICAL INC., Novato, CA (US)

(72) Inventor: Peter COLOSI, Novato, CA (US)

(21) Appl. No.: 17/777,690

(22) PCT Filed: Nov. 13, 2020
(86) PCT No.: PCT/US2020/060337

§ 371 Date: May 18, 2022

Related U.S. Application Data
(60) Provisional application No. 63/016,365, filed on Apr. 28, 2020.
(60) Provisional application No. 62/935,359, filed on Nov. 14, 2019.

(51) Int. Cl. C07K 14/81; C12N 15/86; A61K 48/00; A61P 7/10; A61K 45/06

(52) U.S. Cl. CPC C07K 14/8121; A61K 45/06; A61K 48/0058; A61K 48/0066; A61P 7/10; C12N 15/86

(57) ABSTRACT

Provided herein are compositions and methods for treating a Cl esterase inhibitor deficiency by normalizing levels of the Cl esterase inhibitor protein in a subject having HAE.

### FIELD

Provided herein are recombinant adeno-associated virus (rAAV) vectors and virus particles for treating hereditary angioedema by achieving long term expression of C1 esterase inhibitor (C1EI, or C1-INH) in the liver of a subject.

### BACKGROUND

Hereditary angioedema (HAE) is caused by mutations in the C1EI gene, which is referred to as SERPING1. Most patients (85%) have low levels of C1EI (known as Type I HAE), while a minority (15%) have normal or elevated levels of mutated C1EI that is dysfunctional (known as Type II HAE). There is a third type of HAE (Type III HAE) in which patients have normal C1EI protein but have a mutation in other genes, such as the Factor XII gene, which causes the HAE. Type I and II HAE, characterized by a deficiency in functional plasma C1 esterase inhibitor, can result in inflammatory crises due to unregulated activation of the complement pathway and/or contact activation pathway. The crises present with symptoms of urticaria and/or angioedema, such as swelling of the skin and/or mucous membranes (subcutaneous edema or submucosal edema), including the respiratory and gastrointestinal tracts. Swelling of the larynx can cause fatal asphyxiation. The recurrent episodes of severe swelling can affect arms, legs, face, intestinal tract and airway which are painful, disfiguring and, sometimes, life threatening if they obstruct respiration. If left untreated, the condition has a 25% mortality rate. HAE is estimated to affect 1 in 50,000-100,000 individuals globally.

HAE crises can be triggered by minor surgical or dental procedures or trauma, infection, stress, and the use of medications, especially inhibitors of angiotensin-converting enzyme (ACE) and estrogens. Acute crises are typically treated with C1EI protein, fresh frozen plasma, plasma-derived C1EI protein, ecallantide (a kallikrein inhibitor) and/or icatibant (a bradykinin B2 receptor antagonist). Conventional prophylactic therapy includes plasma-derived C1EI protein, attenuated androgens such as danazol, antifibrinolytic agents and progesterone, although each of these has adverse effects. Treatment of pregnant women represents a problem because androgens and antifibinolytic agents are contraindicated during pregnancy.

C1EI is a serine protease inhibitor, which directly or indirectly inhibits several proteases associated with HAE attacks. It is a major inhibitor of several complement proteases such as C1r and C1s and contact proteases, including factor XIIa and kallikrein, and a minor inhibitor of fibrinolytic proteases such as plasmin and factor XIa. In patients with HAE, uninhibited activation of the contact pathway due to insufficient levels of functional C1-INH results in unregulated cleavage of high molecular weight kininogen by kallikrein, leading to generation of excessive free bradykinin, a potent vasoactive peptide that increases capillary permeability and edema. See, e.g., Riedl M. “Recombinant human C1 esterase inhibitor in the management of hereditary angioedema.” Clin Drug Investig. 2015;35(7):407-417.

### SUMMARY

The embodiments described herein relate to a vector construct, a recombinant replication deficient AAV particle, cells, and pharmaceutical compositions for delivering functional human C1 esterase inhibitor (C1EI) to a subject in need thereof, particularly a subject with hereditary angioedema, or a deficiency in functional C1EI. The embodiments described herein also relate to the use of such AAV particles or such vector constructs to deliver a gene encoding human C1EI to liver cells of patients (human subjects) diagnosed with hereditary angioedema, or a deficiency in functional C1EI.

In one aspect, the embodiments described herein provide a vector construct comprising a nucleic acid sequence that encodes a functional C1 esterase inhibitor (C1EI). In one or more embodiments, the functional C1EI comprises an amino acid sequence at least 90%, 95% or 98% identical to amino acids 23-500 of SEQ ID NO: 2 (a human C1EI, or “hC1EI”). In example embodiments, the nucleic acid sequence encoding the functional C1EI is a wild-type sequence, of which SEQ ID NO: 1 is one example, or is codon optimized, or is a variant. Alternative codon optimized human C1EI-encoding sequences are set out in SEQ ID NOs: 10-13, 59 or 60. In example embodiments, the nucleic acid sequence encoding the functional C1EI is comprises a nucleotide sequence having at least 90% homology to at least 100, 200, 300, 400, or 500 consecutive bases of SEQ ID NO: 1 or 10-13 or 59-60, and which encodes functional human C1 esterase inhibitor (hC1EI) at least 95% identical to amino acids 23-500 of SEQ ID NO: 2. The coding sequence for hC1EI is, in some embodiments, codon optimized for expression in humans. In one embodiment, the codon optimized hC1EI nucleic acid comprises a reduced CpG di-nucleotide content. In a specific embodiment, the CpG di-nucleotide content is less than 25.

In one or more embodiments, the nucleic acid sequence encoding C1EI is operably linked to one or more heterologous expression control elements. Preferably, expression of the hC1EI-encoding transgene is controlled by liver-specific expression control elements. Thus, in such embodiments, in the vector constructs described herein, the nucleic acid sequence encoding C1EI is operably linked to a heterologous liver-specific transcription regulatory region. In some embodiments, in the vector constructs described herein, the expression control elements include one or more of the following: a promoter and/or enhancer; optionally an intron; and a polyadenylation (polyA) signal. Such elements are further described herein.

The liver-specific transcription regulatory region may comprise one or more liver-specific expression control elements. In one or more embodiments, the liver-specific transcription regulatory region is a synthetic promoter sequence comprising portions of a human alpha-1-antitrypsin (hAAT) promoter, a hepatic control region (HCR) enhancer, and/or an apolipoprotein E (ApoE) enhancer. In some embodiments, the liver-specific transcription regulatory region comprises (a) a shortened ApoE enhancer sequence at least 90% identical to SEQ ID NO: 16; (b) an alpha anti-trypsin (hAAT) proximal promoter sequence at least 90% identical to SEQ ID NO: 3, (c) one or more enhancers selected from the group consisting of (i) an ApoE/HCR enhancer at least 90% identical to SEQ ID NO: 4, (ii) an AAT promoter distal X region, and (iii) an AAT promoter distal region. In an example embodiment, the sequence of the liver-specific transcription regulatory region comprises a nucleotide sequence at least 80%, 85%, 90% or 95% identical to SEQ ID NO: 5. In some embodiments, the liver-specific transcription regulatory region comprises (a) an α-microglobulin enhancer sequence at least 90% identical to SEQ ID NO: 17, and/or (b) an alpha anti-trypsin (AAT) proximal promoter at least 90% identical to SEQ ID NO: 3.

In some embodiments, the vector construct comprises one or more introns. In some embodiments, the intron also enhances expression of the C1EI-encoding nucleic acid, and optionally enhances expression in the liver. In one or more embodiments, the intron is a composite hAAT/hemoglobin intron sequence. In an example embodiment, the intron comprises a nucleotide sequence at least 80%, 85%, 90% or 95% identical to SEQ ID NO: 6, or a nucleotide sequence at least 80%, 85%, 90% or 95% identical to SEQ ID NO: 61. In some embodiments, the nucleic acid sequence that encodes the C1EI comprises the intron.

In some embodiments, the vector construct comprises a polyadenylation signal, optionally a bovine growth hormone (bGH) polyA signal (e.g., SEQ ID NO: 19) or preferably a human growth hormone (hGH) polyA signal (e.g., SEQ ID NO: 7).

The vector construct is preferably a recombinant AAV vector construct. In some embodiments, the vector construct comprises (a) one or both of (i) an AAV 5′ inverted terminal repeat (ITR) and (ii) an AAV3′ ITR; (b) a promoter and/or enhancer, e.g. a liver-specific transcription regulatory region; and (c) a nucleic acid sequence encoding a functionally active human C1 esterase inhibitor protein, or a fragment thereof. In some embodiments, the vector construct comprises (a) an AAV 5′ inverted terminal repeat (ITR) sequence (e.g., SEQ ID NO: 54); (b) a promoter and/or enhancer, e.g. a liver-specific transcription regulatory region; (c) a nucleic acid sequence encoding a functionally active human C1 esterase inhibitor protein; and (d) an AAV 3′ ITR (e.g., SEQ ID NO: 55). The AAV 5′ ITR and/or AAV 3′ ITR may be from a heterologous AAV pseudotype (which may or may not be modified as known in the art). In some embodiments, the 5′ ITR and 3′ ITR sequences are derived from AAV2. In one or more embodiments, the vector construct is an AAV vector genome about 3 kb to about 5 kb in size, or about 2.7 kb to about 4 kb in size. In one or more embodiments, the vector construct is an AAV vector genome about 2.7 kb to about 3.3 kb in size, or about 3.7kb to about 4.1 kb in size, e.g., SEQ ID NOs 57 and 58.

In example embodiments, the vector construct comprises a nucleotide sequence at least 80%, 85%, 90% or 95% identical to any one of SEQ ID NOs: 9, 20-36 or 57-58.

In another aspect, provided herein is a recombinant adeno-associated virus (rAAV) particle comprising an AAV capsid and the vector construct as described in one or more of the embodiments herein. In some embodiments, the recombinant AAV (rAAV) particle used for delivering the C1EI-encoding gene (“rAAV.SERPIN G1” or “AAV-SERPIN G1”) has tropism for the liver. In such embodiments, the rAAV comprises an AAV capsid with liver tropism, for example, an AAV5 capsid at least 90% identical to SEQ ID NO: 46, or a simian AAV capsid, optionally a baboon-derived AAV capsid, or a variant thereof, that exhibits liver tropism. In one or more embodiments, the AAV capsid is a capsid for which preexisting humoral immunity is similar to AAV5, or reduced compared to AAV5, e.g., when evaluated by IVIG neutralization in vitro.

In another aspect, provided herein are methods for the production of a AAV particle, useful as a gene delivery vector, the method comprising the steps of: (1) providing an insect cell comprising one or more nucleic acid constructs (a) comprising a vector construct as described herein comprising a nucleic acid as described herein that is flanked by two AAV ITR nucleotide sequences; (b) a nucleotide sequence encoding one or more AAV Rep proteins which is operably linked to a promoter that is capable of driving expression of the Rep protein(s) in an insect cell (c) a nucleotide sequence encoding one or more AAV capsid proteins which is operably linked to a promoter that is capable of driving expression of the capsid protein(s) in the insect cell; wherein (b) and (c) are in the same expression cassette or in two different expression cassettes; and (d) optionally genes encoding AAP and MAAP contained in the VP⅔; (2) culturing the insect cell defined in (1) under conditions conducive to the expression of the Rep and the capsid proteins; and, optionally (3) recovering the AAV particle.

In yet another aspect, provided herein are pharmaceutical compositions comprising the vector construct described herein or the rAAV particle described herein, and a sterile pharmaceutically acceptable diluent, excipient or carrier.

In a further aspect, provided herein are methods of delivering a C1EI gene to a mammalian subject. Such methods include methods of expressing C1EI in a mammalian subject comprising administering to the subject a composition comprising the vector construct described herein, the rAAV particle described herein, or the pharmaceutical composition described herein, thereby expressing the encoded C1EI protein in the subject. Preferably, in such methods, the mammal is a human and the C1EI is functional human C1EI as described herein. Such methods include a method of expressing C1EI in the liver of a mammal by administering an amount of the vector construct, rAAV particle or pharmaceutical composition effective to increase the level of C1EI expression in the liver of the mammal. Such methods also include a method of increasing the level of functional C1EI in the blood of a mammal by administering an amount of the vector construct, rAAV particle or pharmaceutical composition effective to increase the level of functional C1EI in the blood of a mammal. Such methods also include a method of treating a deficiency in functional C1EI in a mammal by administering an amount of the vector construct, rAAV particle or pharmaceutical composition effective to increase the level of functional C1EI in the blood of a mammal. In some embodiments, the amount of the vector construct, rAAV particle or pharmaceutical composition is effective to increase the level of functional C1EI in blood to about 0.4 IU/ml or 1 IU/ml or higher, or the level of C1EI to about 16 mg/dL or higher.

Such methods also include a method of treating hereditary angioedema in a mammal, or treating or preventing any symptom thereof, comprising administering a therapeutically effective amount of the vector construct, rAAV particle or pharmaceutical composition. In one or more embodiments, such methods reduce the frequency or severity of submucosal or subcutaneous edema in the mammal, acute HAE attacks, or amount of on-demand therapy administered to treat the acute HAE attacks.

In any of the methods described herein, the rAAV particle is delivered at a dose of about 1 × 10¹² to about 1 × 10¹⁴ vg/kg or 1 × 10¹⁵ vg/kg, or alternatively about 2 × 10¹² to about 6 × 10¹³ vg/kg, or alternatively about 6 × 10¹³ vg/kg to about 1 × 10¹⁵ vg/kg.

in an aqueous suspension. In any of the methods described herein, the administration of the vector construct, rAAV particle, or pharmaceutical composition may further comprise administration of prophylactic or therapeutic corticosteroid treatment, and/or may further include administration of a second therapy for treating HAE. In any of the methods herein, prior to administration of an AAV particle to a patient as described above, the prospective patient may be assessed for the presence of anti-AAV capsid antibodies or anti-AAV neutralizing antibodies that are capable of blocking cell transduction or otherwise reduce the overall efficiency of the treatment.

Other embodiments will be evident to one skilled in the art upon reading the present specification.

### BRIEF DESCRIPTION OF DRAWINGS

FIGS. 1A-1C depict schematics of the organization of a variety of vector constructs.

FIG. 2 depicts Serpin G1 ELISA results from transient transfection of HepG2 cells.

FIGS. 3A&B depict human C1EI levels in the blood of mice administered AAV particles comprising the vector construct ApoE/HCR-hAAT.hhI.SERPIN G1.hGH (HAE15) (FIG. 3A) in Example 2. Two different AAV capsids were exemplified, an AAV5 type capsid (greater than 90% identical to SEQ ID NO: 46) and a baboon-derived AAV capsid AAVBba49 (greater than 90% identical to SEQ ID NO: 56). FIG. 3B Illustrates the Serpin G1 ELISA results by individual animals/group. Mice were treated with a dose of AAV5 HAE15 2e14vg/kg or AAVBba49 HAE15 2e14vg/kg.

FIGS. 4A&B depict functional human C1EI levels in the same mice.

FIG. 5 depicts the body weight of the same mice.

FIG. 6 depicts Alanine aminotransferase (ALT) activity in plasma from mice treated with AAV5-HAE15 or Bba49-HAE15.

FIGS. 7A&B depict the hepatic expression of human C1 inhibitor in the liver of mice treated with AAV5-HAE15 or Bba49-HAE15 (FIG. 7A) and measurements of the % C1 Inhibitor(+) hepatocytes (FIG. 7B).

FIGS. 8A&B depict HAE15 DNA and RNA levels, respectively, in the liver as measured by qPCR.

FIGS. 9A&B depict human C1EI protein (FIG. 9A) and functional human C1EI (FIG. 9B) levels in the blood of mice administered various doses of an AAV5 type particle comprising vector construct ApoE/HCR-hAAT.hhI.SERPIN G1.hGH (HAE15) in Example 3. Mice were treated with four different doses of AAV5-HAE15: 6e13vg/kg; 2e13vg/kg; 6e12vg/kg; or 2e12vg/kg (first cohort).

FIGS. 10A-10B depict total human C1EI protein concentrations in plasma (mg/mL) and functional human C1EI protein concentrations in plasma (international units, IU/mL), respectively, for mice treated with five different doses of AAV5-HAE15: 2e14, 6e13vg/kg; 2e13vg/kg; 6e12vg/kg; or 2e12vg/kg (second cohort) in Example 3, through week 52.

FIG. 11 depicts ALT levels (IU/L) for the second cohort, through week 52.

FIG. 12 depicts the amount of vector-induced human SERPING1 DNA in liver (copies of DNA per µg of DNA), at week 12 for the first cohort, or at week 52 for the second cohort.

FIG. 13 depicts the percentage of hepatocytes positive for C1EI expression by immunohistochemistry, for the first cohort (at 12 weeks) and the second cohort (at 52 weeks). FIG. 13 also includes for comparison purposes (far left) the data from AAV5-HAE15 administration in Example 2.

FIG. 14 depicts functional human C1EI levels (IU/mL) in plasma over 6 weeks in an animal model of HAE (homozygous SERPING1-/- mice) treated with 6e13vg/kg; 2e13vg/kg; or 6e12vg/kg AAV5-HAE15.

FIGS. 15A, 15B and 15C depict the amount of Evan’s blue dye that accumulated in the ear pinna, small intestine, and kidney, respectively, of homozygous SERPING1-/- mice treated with 6e13vg/kg, 2e13vg/kg, or 6e12vg/kg AAV5-HAE15, as assessed by OD600 (optical density/tissue weight). The amount of blue dye detected correlates to vascular permeability in this animal model of HAE.

### DETAILED DESCRIPTION

Provided herein are nucleic acids or vector constructs encoding functionally active therapeutic C1EI proteins, AAV vector genomes and replication deficient rAAV particles comprising such vector constructs, and pharmaceutical compositions comprising such vector constructs, vector genomes and AAV particles. The compositions and methods of the invention may provide improved AAV virus production yield and/or simplified purification and/or enhanced expression, particularly enhanced liver-specific expression. Also provided herein are methods of making the vector constructs, AAV vector genomes and replication deficient rAAV particles comprising such vector constructs. Further provided herein are methods of treating a deficiency in functional C1EI, or hereditary angioedema.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present disclosure, the following terms are defined below.

As used herein, in the context of gene delivery, the term “vector” or “gene delivery vector” may refer to a particle that functions as a gene delivery vehicle, and which comprises nucleic acid (i.e., the vector genome comprising any of the vector constructs described herein) packaged within, for example, an envelope or capsid. A gene delivery vector may be a viral gene delivery vector or a non-viral gene delivery vector. Alternatively, in some contexts, the term “vector” may be used to refer only to the vector genome or vector construct. Viral vectors suitable for use herein may be a parvovirus, an adenovirus, a retrovirus, a lentivirus or a herpes simplex virus. The parvovirus may be an adenovirus-associated virus (AAV).

As used herein, the term “AAV” is a standard abbreviation for adeno-associated virus. Adeno-associated virus is a single-stranded DNA parvovirus that grows only in cells in which certain functions are provided by a co-infecting helper virus. There are numerous serotypes of AAV that have been characterized. General information and reviews of AAV can be found in, for example, Carter, 1989, Handbook of Parvoviruses, Vol. 1, pp. 169-228; and Berns, 1990, Virology, pp. 1743-1764, Raven Press, (New York); Gao et al., 2011, Methods Mol. Biol. 807: 93-118; Ojala et al., 2018, Mol. Ther. 26(1): 304-19. However, it is fully expected that these same principles will be applicable to additional AAV serotypes since it is well known that the various serotypes are quite closely related, both structurally and functionally, even at the genetic level. (See, e.g., Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs).

As used herein, an “AAV vector construct” refers to nucleic acids, either single-stranded or double-stranded, having at least one of (i) an AAV 5′ inverted terminal repeat (ITR) sequence and (ii) an AAV 3′ ITR flanking a protein-coding sequence (in one embodiment, a functional therapeutic protein-encoding sequence, e.g. C1EI) operably linked to transcription regulatory elements (also called “expression control elements”) that are heterologous to protein-encoding sequence and/or heterologous to the AAV viral genome, i.e., one or more promoters and/or enhancers and, optionally, a polyadenylation sequence and/or one or more introns inserted between exons of the protein-coding sequence. A single-stranded AAV vector refers to nucleic acids that are present in the genome of an AAV virus particle, and can be either the sense strand or the anti-sense strand of the nucleic acid sequences disclosed herein. The size of such single-stranded nucleic acids is provided in bases. A double-stranded AAV vector refers to nucleic acids that are present in the DNA of plasmids, e.g., pUC19, or genome of a double-stranded virus, e.g., baculovirus, used to express or transfer the AAV vector nucleic acids. The size of such double-stranded nucleic acids in provided in base pairs (bp).

The AAV vector constructs provided herein in single strand form are less than about 7.0 kb in length, or are less than 6.5 kb in length, or are less than 6.4 kb in length, or are less than 6.3 kb in length, or are less than 6.2 kb in length, or are less than 6.0 kb in length, or are less than 5.8 kb in length, or are less than 5.6 kb in length, or are less than 5.5 kb in length, or are less than 5.4 kb in length, or are less than 5.3 kb in length, or are less than 5.2 kb in length or are less than 5.0 kb in length, or are less than 4.8 kb in length, or are less than 4.6 kb in length, or are less than 4.5 kb in length, or are less than 4.4 kb in length, or are less than 4.3 kb in length, or are less than 4.2 kb in length, or are less than 4.1 kb in length, or are less than 4.0 kb in length, or are less than 3.9 kb in length, or are less than 3.8 kb in length, or are less than 3.7 kb in length, or are less than 3.6 kb in length, or are less than 3.5 kb in length, or are less than 3.4 kb in length, or are less than 3.3 kb in length, or are less than 3.2 kb in length, or are less than 3.1 kb in length, or are less than 3.0 kb in length. The AAV vector constructs provided herein in single strand form range from about 5.0 kb to about 6.5 kb in length, or range from about 4.8 kb to about 5.2 k in length, or 4.8 kb to 5.3 kb in length, or range from about 4.9 kb to about 5.5 kb in length, or about 4.8 kb to about 6.0 kb in length, or about 5.0 kb to 6.2 kb in length or about 5.1 kb to about 6.3 kb in length, or about 5.2 kb to about 6.4 kb in length, or about 5.5 kb to about 6.5 kb in length, or range from about 4.0 kb to about 5.0 kb in length, or range from about 3.8 kb to about 4.8 k in length, or 3.6 kb to 4.6 kb in length, or range from about 3.4 kb to about 4.4 kb in length, or range from about 3.2 kb to about 4.2 kb in length, or range from about 3.0 kb to 4.0 kb in length, or range from about 3.5 kb to about 4.0 kb in length, or range from about 3.0 kb to about 3.5 kb in length.

While AAV particles have been reported in the literature having AAV genomes of > 5.0 kb, in many of these cases the 5′ or 3′ ends of the encoded genes appear to be truncated (see Hirsch et al., Molec. Ther. 18:6-8, 2010, and Ghosh et al., Biotech. Genet. Engin. Rev. 24:165-178, 2007). It has been shown, however, that overlapping homologous recombination occurs in AAV infected cells between nucleic acids having 5′ end truncations and 3′ end truncations so that a “complete” nucleic acid encoding the large protein is generated, thereby reconstructing a functional, full-length gene.

Oversized AAV vectors are randomly truncated at the 5′ ends and lack a 5′ AAV ITR. Because AAV is a single-stranded DNA virus, and packages either the sense or antisense strand, the sense strand in oversized AAV vectors lacks the 5′ AAV ITR and possibly portions of the 5′ end of the target protein-coding gene, and the antisense strand in oversized AAV vectors lacks the 3′ ITR and possibly portions of the 3′ end of the target protein-coding gene. A functional transgene is produced in oversized AAV vector infected cells by annealing of the sense and antisense truncated genomes within the target cell. Thus, in certain embodiments, the AAV C1EI vectors and/or viral particles comprise at least one ITR.

The term “inverted terminal repeat (ITR)” as used herein refers to the art-recognized regions found at the 5′ and 3′ termini of the AAV genome which function in cis as origins of DNA replication and as packaging signals for the viral genome. AAV ITRs, together with the AAV rep coding region, provide for efficient excision and rescue from, and integration of a nucleotide sequence interposed between two flanking ITRs into a host cell genome. Sequences of certain AAV-associated ITRs are disclosed by Yan et al., J. Virol. (2005) vol. 79, pp. 364-379 which is herein incorporated by reference in its entirety. ITR sequences that find use herein may be full length, wild-type AAV ITRs or fragments thereof that retain functional capability, or may be sequence variants of full-length, wild-type AAV ITRs that are capable of functioning in cis as origins of replication. AAV ITRs useful in the recombinant AAV C1EI vectors of the embodiments provided herein may be derived from any known AAV serotype and, in certain embodiments, derived from the AAV2 or AAV5 serotype.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A “transcription regulatory element” refers to nucleotide sequences of a gene involved in regulation of genetic transcription including a promoter, plus response elements, activator and enhancer sequences for binding of transcription factors to aid RNA polymerase binding and promote expression, and operator or silencer sequences to which repressor proteins bind to block RNA polymerase attachment and prevent expression. The term “liver specific transcription regulatory element” or “liver-specific transcription regulatory region” refers to a regulatory element or region that produces preferred gene expression specifically in the liver tissue. Examples of liver specific regulatory elements include, but are not limited to, the mouse thyretin promoter (mTTR), the endogenous human factor VIII promoter (F8), human apolipoprotein E hepatic control region and active fragments thereof, human alpha-1-antitrypsin promoter (hAAT) and active fragments thereof, human alpha-1-microglobulin promoter and fragments thereof, human prothrombin promoter and active fragments thereof, human albumin minimal promoter, and mouse albumin promoter. Enhancers derived from liver-specific transcription factor binding sites are also contemplated, such as EBP, DBP, HNF1, HNF3, HNF4, HNF6, and Enh1.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

In one embodiment, the vector construct comprises a nucleic acid encoding a functionally active C1EI protein. The C1EI encoding sequence may be wild-type, codon optimized, or a variant.

As used herein, wild-type SERPIN G1 (C1EI-encoding gene) has the following nucleic acid sequence:

ATGGCCTCCAGGCTGACCCTGCTGACCCTCCTGCTGCTGCTGCTGGCTGG
GGATAGAGCCTCCTCAAATCCAAATGCTACCAGCTCCAGCTCCCAGGATC
CAGAGAGTTTGCAAGACAGAGGCGAAGGGAAGGTCGCAACAACAGTTATC
TCCAAGATGCTATTCGTTGAACCCATCCTGGAGGTTTCCAGCTTGCCGAC
AACCAACTCAACAACCAATTCAGCCACCAAAATAACAGCTAATACCACTG
ATGAACCCACCACACAACCCACCACAGAGCCCACCACCCAACCCACCATC
CAACCCACCCAACCAACTACCCAGCTCCCAACAGATTCTCCTACCCAGCC
CACTACTGGGTCCTTCTGCCCAGGACCTGTTACTCTCTGCTCTGACTTGG
AGAGTCATTCAACAGAGGCCGTGTTGGGGGATGCTTTGGTAGATTTCTCC
CTGAAGCTCTACCACGCCTTCTCAGCAATGAAGAAGGTGGAGACCAACAT
GGCCTTTTCCCCATTCAGCATCGCCAGCCTCCTTACCCAGGTCCTGCTCG
GGGCTGGGGAGAACACCAAAACAAACCTGGAGAGCATCCTCTCTTACCCC
AAGGACTTCACCTGTGTCCACCAGGCCCTGAAGGGCTTCACGACCAAAGG
TGTCACCTCAGTCTCTCAGATCTTCCACAGCCCAGACCTGGCCATAAGGG
ACACCTTTGTGAATGCCTCTCGGACCCTGTACAGCAGCAGCCCCAGAGTC
CTAAGCAACAACAGTGACGCCAACTTGGAGCTCATCAACACCTGGGTGGC
CAAGAACACCAACAACAAGATCAGCCGGCTGCTAGACAGTCTGCCCTCCG
ATACCCGCCTTGTCCTCCTCAATGCTATCTACCTGAGTGCCAAGTGGAAG
ACAACATTTGATCCCAAGAAAACCAGAATGGAACCCTTTCACTTCAAAAA
CTCAGTTATAAAAGTGCCCATGATGAATAGCAAGAAGTACCCTGTGGCCC
ATTTCATTGACCAAACTTTGAAAGCCAAGGTGGGGCAGCTGCAGCTCTCC
CACAATCTGAGTTTGGTGATCCTGGTACCCCAGAACCTGAAACATCGTCT
TGAAGACATGGAACAGGCTCTCAGCCCTTCTGTTTTCAAGGCCATCATGG
AGAAACTGGAGATGTCCAAGTTCCAGCCCACTCTCCTAACACTACCCCGC
ATCAAAGTGACGACCAGCCAGGATATGCTCTCAATCATGGAGAAATTGGA
ATTCTTCGATTTTTCTTATGACCTTAACCTGTGTGGGCTGACAGAGGACC
CAGATCTTCAGGTTTCTGCGATGCAGCACCAGACAGTGCTGGAACTGACA
GAGACTGGGGTGGAGGCGGCTGCAGCCTCCGCCATCTCTGTGGCCCGCAC
CCTGCTGGTCTTTGAAGTGCAGCAGCCCTTCCTCTTCGTGCTCTGGGACC
AGCAGCACAAGTTCCCTGTCTTCATGGGGCGAGTATATGACCCCAGGGCC
TGA (SEQ ID NO: 1).

As used herein, wild-type C1-INH (C1EI protein) has the following amino acid sequence:

MASRLTLLTLLLLLLAGDRASSNPNATSSSSQDPESLQDRGEGKVATTVI
SKMLFVEPILEVSSLPTTNSTTNSATKITANTTDEPTTQPTTEPTTQPTI
QPTQPTTQLPTDSPTQPTTGSFCPGPVTLCSDLESHSTEAVLGDALVDFS
LKLYHAFSAMKKVETNMAFSPFSIASLLTQVLLGAGENTKTNLESILSYP
KDFTCVHQALKGFTTKGVTSVSQIFHSPDLAIRDTFVNASRTLYSSSPRV
LSNNSDANLELINTWVAKNTNNKISRLLDSLPSDTRLVLLNAIYLSAKWK
TTFDPKKTRMEPFHFKNSVIKVPMMNSKKYPVAHFIDQTLKAKVGQLQLS
HNLSLVILVPQNLKHRLEDMEQALSPSVFKAIMEKLEMSKFQPTLLTLPR
IKVTTSQDMLSIMEKLEFFDFSYDLNLCGLTEDPDLQVSAMQHQTVLELT
ETGVEAAAASAISVARTLLVFEVQQPFLFVLWDQQHKFPVFMGRVYDPRA
(SEQ ID NO: 2).

The vector constructs described herein may comprise a nucleotide sequence that differs from wild type nucleotide sequence but still encodes a functional C1 esterase inhibitor amino acid sequence at least 90%, 95% or 98% identical to amino acids 23-500 of SEQ ID NO: 2. According to this aspect, the nucleotide sequence may comprise a portion having at least 80%, 85%, or 90% homology to at least 100 consecutive bases of SEQ ID NO: 1 or 10-12, as long as the nucleotide sequence encodes functional human C1 esterase inhibitor at least 90%, 95% or 98% identical to amino acids 23-500 of SEQ ID NO: 2. In example embodiments, the nucleotide sequence may comprise a portion having at least 90% homology to at least 100, 200, 300, 400, or 500 consecutive bases of SEQ ID NO: 1 or 10-12, as long as the nucleotide sequence encodes functional human C1 esterase inhibitor at least 90% identical to amino acids 23-500 of SEQ ID NO: 2. In example embodiments, the nucleotide sequence has substantial homology to the nucleotide sequence of SEQ ID NO: 1 or 10-12 and encodes functional C1EI. The term substantial homology can be further defined with reference to a percent (%) homology, e.g. at least 80%, 85%, 90% or 95% homologous. This is discussed in further detail elsewhere herein.

The term “isolated” when used in relation to a nucleic acid molecule of the present disclosure typically refers to a nucleic acid sequence that is identified and separated from at least one contaminant nucleic acid with which it is ordinarily associated in its natural source. Isolated nucleic acid may be present in a form or setting that is different from that in which it is found in nature. Isolated nucleic acid molecules therefore are distinguished from the nucleic acid molecule as it exists in natural cells.

As used herein, the term “variant” refers to a polynucleotide (or polypeptide) having a sequence substantially similar to a reference polynucleotide (or polypeptide). Procedures for the introduction of nucleotide and amino acid changes in a polynucleotide, protein or polypeptide are known to the skilled artisan (see, e.g., Sambrook et al. (1989)). In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by skilled artisans.

The term “identity,” “homology” and grammatical variations thereof, mean that two or more referenced entities are the same, when they are “aligned” sequences. Thus, by way of example, when two polypeptide sequences are identical, they have the same amino acid sequence, at least within the referenced region or portion. Where two polynucleotide sequences are identical, they have the same polynucleotide sequence, at least within the referenced region or portion. The identity can be over a defined area (region or domain) of the sequence. An “area” or “region” of identity refers to a portion of two or more referenced entities that are the same. Thus, where two protein or nucleic acid sequences are identical over one or more sequence areas or regions they share identity within that region. An “aligned” sequence refers to multiple polynucleotide or protein (amino acid) sequences, often containing corrections for missing or additional bases or amino acids (gaps) as compared to a reference sequence. “Substantial homology” means that a molecule is structurally or functionally conserved such that it has or is predicted to have at least partial structure or function of one or more of the structures or functions (e.g., a biological function or activity) of the reference molecule, or relevant/corresponding region or portion of the reference molecule to which it shares homology.

“Percent (%) nucleic acid sequence identity or homology” is defined as the percentage of nucleotides in a candidate sequence that are identical with a reference sequence after aligning the respective sequences and introducing gaps, if necessary, to achieve the maximum percent sequence identity. Alignment for purposes of determining percent nucleic acid sequence identity can be achieved in various ways that are within the skill in the art, for instance, using publicly available computer software such as ALIGN or Megalign (DNASTAR) software. Those skilled in the art can determine appropriate parameters for measuring alignment, including any algorithms needed to achieve maximal alignment over the full length of the sequences being compared.

“Percent (%) amino acid sequence identity or homology” with respect to the C1EI amino acid sequences identified herein is defined as the percentage of amino acid residues in a candidate sequence that are identical to the amino acid residues in a C1EI polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as ALIGN or Megalign (DNASTAR) software. Those skilled in the art can determine appropriate parameters for measuring alignment, including any algorithms needed to achieve maximal alignment over the full length of the sequences being compared.

“Codon optimization” or “codon optimized” refers to changes made in the nucleotide sequence so that it is more likely to be expressed at a relatively high level compared to the non-codon optimized sequence. It does not change the amino acid for which each codon encodes.

As used herein, an “intron” is broadly defined as a sequence of nucleotides that is removable by RNA splicing. “RNA splicing” means the excision of introns from a pre-mRNA to form a mature mRNA. Introns may be upstream, downstream, or within the coding region of a gene. Insertion of an intron into a nucleotide sequence can be accomplished by any method known in the art. The only limitation of where the intron is inserted is in consideration of the packaging limitations of the AAV virus particles (about 5 kbp).

In certain embodiments, the recombinant AAV vector construct comprises (a) a nucleic acid comprising an AAV2 5′ inverted terminal repeat (ITR) (which may or may not be modified as known in the art), (b) a liver-specific transcription regulatory region, (c) a functional C1EI protein coding region, (d) optionally one or more introns, (e) a polyadenylation sequence, and (f) an AAV2 3′ ITR (which may or may not be modified as known in the art).

Other embodiments provided herein are directed to vector constructs encoding a functional C1EI polypeptide, wherein the constructs comprise one or more of the individual elements of the above described constructs and combinations thereof, in one or more different orientation(s). Another embodiment provided herein is directed to the above described constructs in an opposite orientation. In another embodiment, provided are recombinant AAV virus particles comprising the herein described AAV vector constructs and their use for the treatment of HAE or deficiency in functional C1EI in subjects. In one embodiment the subjects are juvenile subjects.

An “AAV virion” or “AAV viral particle” or “AAV vector particle” or “AAV virus” refers to a viral particle composed of at least one AAV capsid protein and an encapsidated AAV vector construct as described herein. If the particle comprises a heterologous polynucleotide (i.e., a polynucleotide other than a wild-type AAV genome such as a transgene to be delivered to a mammalian cell), it is typically referred to as a “recombinant AAV vector particle” or simply an “AAV vector”. Production of AAV vector particles necessarily includes production of AAV vector genome, as such a vector genome is contained within an AAV vector particle. It is understood that reference to the polynucleotide AAV vector construct encapsulated within the vector particle, and replication thereof, refers to the AAV vector genome.

As used herein “therapeutic AAV virus” refers to an AAV virion, AAV viral particle, AAV vector particle, or AAV virus that comprises a heterologous polynucleotide that encodes a therapeutic protein such as the C1EI described herein. An “AAV vector construct” or “AAV vector genome” as used herein refers to a vector construct comprising one or more polynucleotide encoding a protein of interest (also called transgenes) that are flanked by at least one AAV terminal repeat sequences (ITRs) and operably linked to one or more expression control elements. Such AAV vector constructs can be replicated and packaged into infectious viral particles when present in a host cell that has been transfected with a vector encoding and expressing rep and cap gene products.

As used herein “therapeutic protein” refers to a polypeptide that has a biological activity that replaces or compensates for the loss or reduction of activity of an endogenous protein. For example, a functional C1 esterase inhibitor (C1EI) is a therapeutic protein for hereditary angioedema (HAE).

“Hereditary angioedema (HAE)” as used herein refers to an inherited metabolic disease that is characterized by recurrent attacks or symptoms of subcutaneous and/or submucosal edema (swelling), particularly in the skin, gastrointestinal tract and respiratory tract due to activation of the complement pathway and/or contact activation pathway. The recurrent episodes of severe swelling can affect arms, legs, face, intestinal tract and airway which are painful, disfiguring and, sometimes, life threatening if they obstruct respiration. If left untreated, the condition has a 25% mortality rate.

Type I HAE and Type II HAE are caused by a deficiency of functional C1 esterase inhibitor (C1EI) protein. Type I HAE is characterized by low expression levels of C1EI. Type II HAE is characterized by normal or elevated expression levels of a non-functional C1EI. Type III HAE is characterized by normal levels of functional C1EI but a mutation in other genes such as Factor XII.

“C1 esterase inhibitor (C1EI) deficiency” or a “deficiency in functional C1EI” as used herein refers to an inherited condition caused by a deficiency of functional C1 esterase inhibitor (C1EI) protein. This includes Type I and Type II HAE. The uninhibited activation of the complement and/or contact activation pathway due to insufficient levels of functional C1EI results in unregulated cleavage of high molecular weight kininogen by kallikrein, leading to generation of excessive free bradykinin, a potent vasoactive peptide which increases capillary permeability and edema .

“Therapeutically effective for HAE” or “HAE therapy” as used herein refers to any therapeutic intervention of a subject having HAE that ameliorates HAE symptoms or reduces the frequency, duration or severity of acute HAE attacks, or reduces the amount of on-demand therapy (e.g. human C1EI protein, kallikrein inhibitor, bradykinin antagonist, etc.) required to treat acute HAE attacks, or reduces the frequency with which on-demand therapy is administered to treat acute HAE attacks.. “HAE gene therapy” as used herein refers to any therapeutic intervention of a subject having HAE that involves the replacement or restoration or increase of C1EI activity through the delivery of one or more nucleic acid molecules to the cells of the subject that express functional C1EI protein. In certain embodiments, HAE gene therapy refers to gene therapy involving an adeno associated viral (AAV) particle comprising a vector construct that expresses human C1EI.

“Treat” or “treatment” as used herein refers to therapeutic treatment which refers to a treatment administered to a subject who exhibits signs or symptoms of pathology, i.e., HAE, for the purpose of diminishing or eliminating those signs or symptoms. The signs or symptoms can be biochemical, cellular, histological, functional, subjective or objective. “Treat” or “treatment” or “therapeutically effective” refers to the reduction or amelioration of the progression, severity, and/or duration of a disease (or symptom related thereto) associated with C1EI deficiency or HAE, e.g. frequency or severity of subcutaneous edema and/or submucosal edema, or abnormally elevated bradykinin levels. Treatment can occur before or after the edema. Reduced symptoms can occur with treatment that restores normal levels of functional C1EI in blood, e.g. about 16 mg/dL (about 1 IU/ml) to about 32 mg/dL, or that restores about 40% or more of normal C1EI levels which may be expected to ameliorate HAE symptoms. See, e.g., Zuraw et al., Allergy 2015; 70: 1319-1328, suggesting that clinically meaningful effects are seen with about 40% of normal levels of C1EI. Treatment preferably is stable treatment that restores functional C1EI to therapeutically effective levels for a clinically significant length of time.

“Ameliorate” as used herein refers to the action of lessening the severity of symptoms, progression, or duration of a disease.

As used herein “stably treating” or “stable treatment” refers to using a therapeutic vector construct, AAV particle or cell administered to a subject where the subject stably expresses a therapeutic protein expressed by the vector construct, AAV particle or cell. Stably expressed therapeutic protein means that the protein is expressed for a clinically significant length of time. “Clinically significant length of time” as used herein means expression at therapeutically effective levels for a length of time that has a meaningful impact on the quality of life of the subject. In certain embodiments a meaningful impact on the quality of life is demonstrated by the lack of a need to administer alternative therapies intravenously or subcutaneously. In certain embodiments clinically significant length of time is expression for at least six months, for at least eight months, for at least one year, for at least two years, for at least three years, for at least four years, for at least five years, for at least six years, for at least seven years, for at least eight years, for at least nine years, for at least ten years, or for the life of the subject. Preferably, therapeutically effective expression continues for at least a year.

As used herein, the term “effective amount” refers to an amount sufficient to effect beneficial or desirable biological and/or clinical results.

As used herein, a “subject” refers to an animal that is the object of treatment, observation or experiment. “Animal” includes cold- and warm-blooded vertebrates and invertebrates such as fish, shellfish, reptiles, and in particular, mammals. The term “avian” as used herein includes, but is not limited to, chickens, ducks, geese, quail, turkeys and pheasants. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human.

In general, a “pharmaceutically acceptable carrier” is one that is not toxic or unduly detrimental to cells. Exemplary pharmaceutically acceptable carriers include sterile, pyrogen-free water and sterile, pyrogen-free, phosphate buffered saline. Pharmaceutically acceptable carriers include physiologically acceptable carriers. The term “pharmaceutically acceptable carrier” includes any and all solvents, dispersion media, coatings, antibacterial and antifungal agents, isotonic and absorption delaying agents, and the like that are physiologically compatible.

In another embodiment, provided are methods of producing recombinant adeno-associated virus (AAV) particles comprising any of the AAV vector constructs provided herein. The methods comprise the steps of culturing a cell that has been transfected with any of the AAV vector constructs provided herein (in association with various AAV cap and rep genes) and recovering recombinant therapeutic AAV particles from the supernatant of the transfected cell.

The cells useful for recombinant AAV production provided herein are any cell type susceptible to baculovirus infection, including insect cells such as High Five, Sf9, Se301, SeIZD2109, SeUCR1, Sf9, Sf900+, Sf21, BTI-TN-5B1-4, MG-1, Tn368, HzAm1, BM-N, Ha2302, Hz2E5, and Ao38. In another embodiment, mammalian cells such as HEK293, HeLa, CHO, NSO, SP2/0, PER.C6, Vero, RD, BHK, HT 1080, A549, Cos-7, ARPE-19, and MRC-5 can be used.

In another embodiment, provided herein is the use of an effective amount of vector nucleic acid, vector construct, or AAV particle for the preparation of a medicament for the treatment of a subject suffering from HAE or C1EI deficiency. In one embodiment, the subject suffering from HAE is a human. In one embodiment, the medicament is administered by intravenous (IV) administration. In another embodiment, administration of the medicament results in expression of C1EI protein in the bloodstream of the subject sufficient to increase levels of functional C1EI protein in the blood in the subject, to ameliorate HAE symptoms. In certain embodiments, the medicament is also for co-administration with a prophylactic and/or therapeutic corticosteroid for the prevention and/or treatment of any hepatotoxicity associated with administration of the AAV-C1EI virus. The prophylactic or therapeutic corticosteroid treatment may comprise at least 5, 10, 15, 20, 25, 30, 35, 40, 45, 50, 55, 60, or more mg/day of the corticosteroid. In certain embodiments, the prophylactic or therapeutic corticosteroid may be administered over a continuous period of at least about 3, 4, 5, 6, 7, 8, 9, 10 weeks, or more.

In another embodiment, the HAE therapy provided herein optionally further includes administration, e.g. concurrent administration, of other therapies that are used to treat HAE, for example an attenuated androgen such as danazol, stanozolol, oxandrolone, methyltestosterone, tibolone, oxymetholone. In some embodiments, the HAE therapy provided herein comprises adjunct administration of one or more of the following: a C1EI protein, optionally recombinant or plasma-derived, a kallikrein inhibitor, a bradykinin antagonist, and/or an attenuated androgen, for acute HAE attacks.

### Vector Constructs and AAV Vectors

The recombinant vector construct of the disclosure may be used itself as gene therapy, or may be used to produce rAAV particles by methods described herein, comprising providing to a suitable host cell the recombinant vector construct, together with Rep and Cap genes. The vector constructs described herein comprise a nucleic acid sequence that encodes a functional C1 esterase inhibitor (C1EI). The recombinant vector construct may comprise a nucleic acid encoding functional human C1EI operably linked to a heterologous expression control element, e.g. a promoter and/or enhancer; optionally an intron; and optionally a polyadenylation (polyA) signal. The heterologous expression control element may be a heterologous liver-specific transcription regulatory region, e.g., as described herein.

When used to produce rAAV particles, the recombinant vector construct may comprise (a) one or both of (i) an AAV 5′ inverted terminal repeat (ITR) sequence and (ii) an AAV 3′ ITR, (b) a heterologous liver-specific transcription regulatory region, and (c) a nucleic acid encoding a functional human C1EI, optionally wherein the AAV ITRs are AAV2 ITRs. Preferably, the nucleic acid encoding the functional C1EI is operably linked to liver-specific expression control elements. The vector construct may include additional expression control elements, for example: a promoter and/or enhancer; an intron; optionally an exon from the same gene as the intron; and a polyadenylation (polyA) signal. Such elements are further described herein. Preferably, the rAAV particles also comprise an AAV capsid with liver tropism, optionally an AAV5 type capsid.

In one or more embodiments, the functional C1EI comprises an amino acid sequence at least 90%, 95% or 98% identical to amino acids 23-500 of SEQ ID NO: 2 (a human C1EI, or “hC1EI”). In example embodiments, the nucleic acid sequence encoding the functional C1EI is a wild-type SERPIN G1 sequence, of which SEQ ID NO: 1 is one example, or is codon optimized, or is a variant.

In one or more embodiments, the nucleic acid sequence encoding C1EI is operably linked to one or more heterologous expression control elements. Preferably, the expression control element is a liver-specific expression control element. Examples of liver specific control elements include, but are not limited to, the mouse thyretin promoter (mTTR), the endogenous human factor VIII promoter (F8), human apolipoprotein E hepatic control region and active fragments thereof, human alpha-1-antitrypsin promoter (hAAT) and active fragments thereof, human alpha-1-microglobulin promoter and fragments thereof, human prothrombin promoter and active fragments thereof, human albumin minimal promoter, and mouse albumin promoter. Enhancers derived from liver-specific transcription factor binding sites are also contemplated, such as EBP, DBP, HNF1, HNF3, HNF4, HNF6, and Enh1.

In some embodiments, in the vector constructs comprise a nucleic acid sequence encoding functional C1EI that is operably linked to a heterologous liver-specific transcription regulatory region. The vector constructs may comprise other regulatory elements. In some embodiments, in the vector constructs described herein, the expression control elements include one or more of the following: a promoter and/or enhancer; optionally an intron; and a polyadenylation (polyA) signal.

The liver-specific transcription regulatory region may comprise one or more liver-specific expression control elements. In one or more embodiments, the liver-specific transcription regulatory region is a synthetic promoter sequence comprising portions of a human alpha-1-antitrypsin (hAAT) promoter, a hepatic control region (HCR) enhancer, and/or an apolipoprotein E (ApoE) enhancer.

In some embodiments, the vector construct comprises at least one or both of a 5′ inverted terminal repeat (ITR) of AAV and a 3′ AAV ITR, a promoter, a nucleic acid encoding functional C1EI, and optionally a posttranscriptional regulatory element, where the promoter, the nucleic acid encoding C1EI and the posttranscription regulatory element are located downstream of the 5′ AAV ITR and upstream of the 3′ AAV ITR. The vector construct can, for example, be used to produce high levels of C1EI in a subject for therapeutic purposes.

In certain embodiments, the recombinant AAV vector construct comprises a nucleic acid comprising (a) an AAV2 5′ inverted terminal repeat (ITR) (which may or may not be modified as known in the art), (b) a liver-specific transcription regulatory region, a functional C1EI protein coding region, (d) optionally one or more introns, (e) a polyadenylation sequence, and (f) an AAV2 3′ ITR (which may or may not be modified as known in the art).

In some embodiments, the liver-specific transcription regulatory region comprises a shortened ApoE enhancer sequence (SEQ ID NO: 16) or a nucleotide sequence at least 80%, 85%, 90%, 95% or 98% identical thereto; a 186 base human alpha anti-trypsin (hAAT) proximal promoter, including 42 bases of the 5′ untranslated region (UTR) (SEQ ID NO: 15) or a nucleotide sequence at least 80%, 85%, 90%, 95% or 98% identical thereto; one or more enhancers selected from the group consisting of (i) a 34 base human ApoE/HCR enhancer (SEQ ID NO: 4) or a nucleotide sequence at least 80%, 85%, 90%, 95% or 98% identical thereto, (ii) a 32 base human AAT promoter distal X region or a nucleotide sequence at least 80%, 85%, 90%, 95% or 98% identical thereto, and (iii) 80 additional bases of distal element of the human AAT proximal promoter or a nucleotide sequence at least 80%, 85%, 90%, 95% or 98% identical thereto; and a nucleic acid encoding human C1EI. In another embodiment, the liver-specific transcription regulatory region comprises an α-microglobulin enhancer sequence (SEQ ID NO: 17) or a nucleotide sequence at least 80%, 85%, 90%, 95% or 98% identical thereto and the 186 base human alpha anti-trypsin (AAT) proximal promoter (SEQ ID NO: 15) or a nucleotide sequence at least 80%, 85%, 90%, 95% or 98% identical thereto.

Other embodiments provided herein are directed to vector constructs encoding a functional C1EI polypeptide, wherein the constructs comprise one or more of the individual elements of the above described constructs and combinations thereof, in one or more different orientation(s). Another embodiment provided herein is directed to the above described constructs in an opposite orientation. In another embodiment, provided are recombinant AAV particles comprising the herein described vector constructs and their use for the treatment of HAE or C1EI deficiency in subjects. In one embodiment the subjects are juvenile subjects.

The AAV vector constructs provided herein in single strand form are less than about 7.0 kb in length, or are less than 6.5 kb in length, or are less than 6.4 kb in length, or are less than 6.3 kb in length, or are less than 6.2 kb in length, or are less than 6.0 kb in length, or are less than 5.8 kb in length, or are less than 5.6 kb in length, or are less than 5.5 kb in length, or are less than 5.4 kb in length, or are less than 5.3 kb in length, or are less than 5.2 kb in length or are less than 5.0 kb in length, or are less than 4.8 kb in length, or are less than 4.6 kb in length, or are less than 4.5 kb in length, or are less than 4.4 kb in length, or are less than 4.3 kb in length, or are less than 4.2 kb in length, or are less than 4.1 kb in length, or are less than 4.0 kb in length, or are less than 3.9 kb in length, or are less than 3.8 kb in length, or are less than 3.7 kb in length, or are less than 3.6 kb in length, or are less than 3.5 kb in length, or are less than 3.4 kb in length, or are less than 3.3 kb in length, or are less than 3.2 kb in length, or are less than 3.1 kb in length, or are less than 3.0 kb in length. The AAV vector constructs provided herein in single strand form range from about 5.0 kb to about 6.5 kb in length, or range from about 4.8 kb to about 5.2 k in length, or 4.8 kb to 5.3 kb in length, or range from about 4.9 kb to about 5.5 kb in length, or about 4.8 kb to about 6.0 kb in length, or about 5.0 kb to 6.2 kb in length or about 5.1 kb to about 6.3 kb in length, or about 5.2 kb to about 6.4 kb in length, or about 5.5 kb to about 6.5 kb in length, or range from about 4.0 kb to about 5.0 kb in length, or range from about 3.8 kb to about 4.8 k in length, or 3.6 kb to 4.6 kb in length, or range from about 3.4 kb to about 4.4 kb in length, or range from about 3.2 kb to about 4.2 kb in length, or range from about 3.0 kb to 4.0 kb in length, or range from about 3.5 kb to about 4.0 kb in length, or range from about 3.0 kb to about 3.5 kb in length. The AAV vector constructs provided herein may also range from about 2.7 kb to about 3.3 kb in length, or about 3.7kb to about 4.1 kb in length, or about 2.7 kb to about 4 kb in length, or about 2.7 kb to about 4.1 kb in length, e.g., SEQ ID NOs 57 (HAE23) and 58 (HAE24). Among the vectors constructs of the disclosure, vector constructs of a smaller size range provide higher expression levels.

When AAV vectors are produced from oversized recombinant vector constructs, they may lack a portion of the 5′ or 3′ ends of the recombinant vector construct. Because AAV is a single-stranded DNA virus, and packages either the sense or antisense strand, the sense strand in oversized AAV vectors lacks the 5′ AAV ITR and possibly portions of the 5′ end of the target protein-coding gene, and the antisense strand in oversized AAV vectors lacks the 3′ ITR and possibly portions of the 3′ end of the target protein-coding gene. A functional transgene is produced in oversized AAV vector infected cells by annealing of the sense and antisense truncated genomes within the target cell. Thus, in certain embodiments, the rAAV particles of the invention may comprise recombinant vector constructs that comprise at least one ITR, and a substantial portion of a nucleotide sequence encoding a functional C1EI, such as a fragment of any of SEQ ID NO: 10-13, 59 or 60 that is greater than 50%, 60%, 70%, 80%, or 90% of the length of the nucleotide sequence. For example, the recombinant vector construct may comprise at least one ITR, a liver-specific transcription regulatory region, and a substantial portion of a nucleotide sequence encoding a functional C1EI. The rAAV particles of the invention may also comprise a substantial portion of any of any one of SEQ NOs: 8, 9, 20-36, 57 and 58 e.g. a fragment that is greater than 50%, 60%, 70%, 80%, or 90% of the length of the nucleotide sequence set forth in any one of SEQ ID NOs: 8, 9, 20-36, 57 and 58, including the liver-specific transcription regulatory region.

Generation of the vector constructs can be accomplished using any suitable genetic engineering techniques well known in the art, including, without limitation, the standard techniques of restriction endonuclease digestion, ligation, transformation, plasmid purification, and DNA sequencing, for example as described in Sambrook et al. (Molecular Cloning: A Laboratory Manual. Cold Spring Harbor Laboratory Press, N.Y. (1989)).

The vector constructs can incorporate sequences from the genome of any known organism. The sequences can be incorporated in their native form or can be modified in any way to obtain a desired activity. For example, the sequences can comprise insertions, deletions or substitutions.

AAV vector constructs can be replicated and packaged into infectious AAV particles, preferably replication deficient AAV particles, when present in a host cell that has been transfected with a polynucleotide encoding and expressing rep and cap gene products.

The vector constructs or AAV particles described herein may also produce beneficial effects in a C1EI-deficient mouse model that shares characteristics associated with HAE in humans including decreased plasma C1EI levels. Phenotypically, these mice have increased vascular permeability of skin and internal organs.

### Transcription Regulatory Elements or Region

### Promoters and Enhancers

Various promoters can be operably linked with a nucleic acid comprising the coding region of the protein of interest, human C1EI, in the vector constructs disclosed herein. In some embodiments, the promoter can drive the expression of the protein of interest in a cell infected with a virus derived from the viral vector, such as a target cell. The promoter can be naturally-occurring or non-naturally occurring. In some embodiments the promoter is a synthetic promoter. In one embodiment the synthetic promoter comprises sequences that do not exist in nature and which are designed to regulate the activity of an operably linked gene. In another embodiment the synthetic promoter comprises fragments of natural promoters to form new stretches of DNA sequence that do not exist in nature. Synthetic promoters are typically comprised of regulatory elements, promoters, enhancers, introns, splice donors and acceptors that are designed to produce enhanced tissue specific expression. Examples of promoters, include, but are not limited to, viral promoters, plant promoters and mammalian promoters. In another embodiment the promoter is a liver specific promoter. Specific examples of liver specific promoters include LP1, HLP, HCR-hAAT, ApoE-hAAT, LSP, TBG and TTR. These promoters are described in more detail in the following references: LP1 (human ApoE HCR core sequence (192 bp) with human AAT promoter (255 bp)): Nathwani A. et al. Blood. 2006 April 1; 107(7): 2653-2661; hybrid liver specific promoter (HLP) (human apolipoprotein E (ApoE) hepatic control region (HCR) fragment (34 bp) with modified human α -1-antitrypsin (αAT) promoter (217 bp)): McIntosh J. et al. Blood. 2013 Apr 25; 121(17): 3335-3344; HCR-hAAT (ApoE-HCR (319 bp) with ApoE enhancer (1-4x154 bp) with human AAT promoter (408 bp) and including an Intron A (1.4 kbp) and 3′UTR (1.7 kbp)): Miao CH et al. Mol Ther. 2000; 1: 522-532; ApoE-hAAT: Okuyama T et al. Human Gene Therapy, 7, 637-645 (1996); LSP: Wang L et al. Proc Natl Acad Sci U S A. 1999 March 30; 96(7): 3906-3910, thyroxine binding globulin (TBG) promoter: Yan et al., Gene 506:289-294 (2012), and transthyretin (TTR) promoter: Costa et al., Mol. Cell. Biol. 8:81-90 (1988)

For example, De Simone et al. (EMBO Journal vol.6 no.9 pp.2759-2766, 1987) describes a number of promoters derived from human α-1-antitrypsin promoter. For example, it characterizes the cis- and trans-acting elements required for liver-specific activity within the human AAT promoter from -1200 to +44. The human AAT promoter in HLP consists of the distal X element (32 bp) and the proximal A and B elements (185 bp). Frain et al. (MOL CELL BIO, March 1990, Vol. 10, No.3, p. 991-999) describes a number of promoters derived from human albumin promoter. For example, it characterizes promoter and enhancer elements within the human albumin gene from -1022 to -1.

Dang et al. (J BIOL CHEM, Vol. 270, No. 38, Issue of September 22, pp. 22577-22585, 1995) describes the hepatic control region (HCR) of human apolipoprotein E gene (774 bp). Shachter et al. (J. Lipid Res. 1993. Vol.34: pp1699-1707) characterizes a liver-specific enhancer in the ApoE HCR (154 bp). These HCR fragments can be combined with other transcription regulatory elements such as the human AAT promoter or fragments thereof. Chow et al. (J Biol Chem. 1991 Oct 5;266(28):18927-33) characterizes the human prothrombin enhancer from -940 to -860 (80 bp). Rouet et al. (Vol. 267, No. 29, Issue of October 15, PP. 20765-20773,1992; Nucleic Acids Res. 1995 Feb 11; 23(3): 395-404; and Biochemical Journal Sep. 15, 1998, 334 (3) 577-584) characterize the sequence of the liver-specific human α-1-microglobulin/bikunin enhancer. U.S. Pat. No. 7,323,324 also describes human AAT promoter, human α-microglobulin/bikunen enhancers, human albumin promoter, and human prothrombin enhancers.

In some embodiments, the promoter comprises the human alpha1 anti-trypsin (hAAT) promoter complex. In some embodiments, the promoter comprises at least a portion of the hAAT promoter. The portion of the hAAT promoter can comprise a nucleic acid sequence having at least about 90%, at least about 95%, at least about 96%, at least about 97%, at least about 98%, at least about 99%, or more, sequence identity to SEQ ID NO: 3.

In some embodiments, the promoter comprises a liver specific enhancer. In some embodiments, the promoter comprises an apolipoprotein E (ApoE) / hepatic control region (HCR) enhancer. In some embodiments, the promoter comprises at least a portion of the ApoE/HCR enhancer. For example, the ApoE/HCR enhancer can comprise a nucleic acid sequence having at least about 90%, at least about 95%, at least about 96%, at least about 97%, at least about 98%, at least about 99%, or more, sequence identity to SEQ ID NO: 4.

In some embodiments, the promoter is a synthetic promoter comprising at least a portion of the hAAT promoter, at least a portion of the ApoE/HCR enhancer. In some embodiments, the promoter can include a nucleic acid sequence having at least about 90%, at least about 95%, at least about 96%, at least about 97%, at least about 98%, at least about 99%, or more, sequence identity to SEQ ID NO: 5.

In some embodiments, the promoter comprises multiple copies of one or more of the enhancers identified above. In some embodiments, the promoter constructs comprise one or more of the individual enhancer elements described above and combinations thereof, in one or more different orientation(s).

In some embodiments, the promoter is operably linked with a polynucleotide encoding one or more proteins of interest. In some embodiments, the promoter is operably linked with a polynucleotide encoding the C1EI protein.

The size of the promoter can vary. Because of the limited packaging capacity of AAV, it is preferred to use a promoter that is small in size, but at the same time allows high level production of the protein(s) of interest in host cells. For example, in some embodiments the promoter is at most about 1.5 kb, at most about 1.4 kb, at most about 1.35 kb, at most about 1.3 kb, at most about 1.25 kb, at most about 1.2 kb, at most about 1.15 kb, at most about 1.1 kb, at most about 1.05 kb, at most about 1 kb, at most about 800 base pairs, at most about 600 base pairs, at most about 400 base pairs, at most about 200 base pairs, or at most about 100 base pairs.

### Other Regulatory Elements

Various additional regulatory elements can be used in the vector constructs, for example enhancers to further increase expression level of the protein of interest in a host cell, a polyadenylation signal, a ribosome binding sequence, and/or a consensus splice acceptor or splice donor site. In some embodiments, the regulatory element can facilitate maintenance of the recombinant DNA molecule extrachromosomally in a host cell and/or improve vector potency (e.g. scaffold/matrix attachment regions (S/MARs)). Such regulatory elements are well known in the art.

The vectors constructs disclosed herein may include regulatory elements such as a transcription initiation region and/or a transcriptional termination region. Examples of a transcription termination region include, but are not limited to, polyadenylation signal sequences. Examples of polyadenylation signal sequences include, but are not limited to, human growth hormone (hGH) poly(A), bovine growth hormone (bGH) poly(A), SV40 late poly(A), rabbit beta-globin (rBG) poly(A), thymidine kinase (TK) poly(A) sequences, and any variants thereof. In some embodiments, the transcriptional termination region is located downstream of the posttranscriptional regulatory element. In some embodiments, the transcriptional termination region is a polyadenylation signal sequence. In some embodiments, the transcriptional termination region is hGH poly(A) sequence (SEQ ID NO:7).

In some embodiments, the vector constructs can include additional transcription and translation initiation sequences, and/or additional transcription and translation terminators, which are known in the art.

### Protein of Interest and Nucleic Acids Encoding the Protein of Interest

As used herein, a “protein of interest” is any functional C1EI protein, including naturally-occurring and non-naturally occurring variants thereof. In some embodiments, a polynucleotide encoding one or more C1EI proteins of interest can be inserted into the viral vectors disclosed herein, wherein the polynucleotide is operably linked with the promoter. In some instances, the promoter can drive the expression of the protein(s) of interest in a host cell (e.g., a human liver cell).

In a first aspect, the present disclosure provides an isolated nucleic acid molecule comprising a nucleotide sequence which encodes functional wild-type C1EI protein (e.g., SEQ ID NO: 2). The nucleotide sequence may be homologous to the wild-type nucleotide sequence of SEQ ID NO: 1.

As described herein, the nucleotide sequence encoding the C1EI protein can be modified to improve expression efficiency of the protein. The methods that can be used to improve the transcription and/or translation of a gene herein are not particularly limited. For example, the nucleotide sequence can be modified to better reflect host codon usage to increase gene expression (e.g., protein production) in the host (e.g., a mammal). As another non-limiting example for the modification, one or more of the splice donors and/or splice acceptors in the nucleotide sequence of the protein of interest is modified to reduce the potential for extraneous splicing. As another non-limiting example for the modification, one or more introns can be inserted within or adjacent to the nucleotide sequence of the protein of interest to optimize AAV vector packaging and enhance expression.

The nucleic acid molecule encodes a functional C1EI protein at least 90% identical to amino acids 23-500 of SEQ ID NO: 2, and preferably at least 95% or 98% identical to a wild type amino acid sequence. If the nucleic acid encodes a protein comprising a sequence having changes to any of the wild-type amino acids, the protein should still be a functional protein. A skilled person will appreciate that minor changes can be made to some of the amino acids of the protein without adversely affecting the function of the protein.

In certain embodiments, the nucleic acid molecule has at least 75%, at least 80%, at least 85%, at least 90%, at least 95% homology, or at least 98% homology to the nucleotide sequence of SEQ ID NO: 1 or 10-12, or at least 100, 200, 300, 400 or 500 consecutive nucleotides of SEQ ID NO: 1 or 10-12. In one embodiment, the nucleic acid molecule encodes for a functional C1EI protein, that is to say it encodes for C1EI which, when expressed, has the functionality of wild type C1EI. In certain embodiments, the nucleic acid molecule, when expressed in a suitable system (e.g. a host cell), produces a functional C1EI protein and at a relatively high level. Since the C1EI that is produced is functional, it will have a conformation which is the same as at least a portion of the wild type C1EI. In certain embodiments, a functional C1EI protein produced as described herein effectively treats a subject suffering from C1EI deficiency and/or HAE.

In another embodiment, the nucleotide sequence coding for a functional C1EI has an improved codon usage bias for the human cell as compared to naturally occurring nucleotide sequence coding for the corresponding non-codon optimized sequence. The adaptiveness of a nucleotide sequence encoding a functional C1EI to the codon usage of human cells may be expressed as codon adaptation index (CAI). A codon adaptation index is herein defined as a measurement of the relative adaptiveness of the codon usage of a gene towards the codon usage of highly expressed human genes. The relative adaptiveness (w) of each codon is the ratio of the usage of each codon, to that of the most abundant codon for the same amino acid. The CAI is defined as the geometric mean of these relative adaptiveness values. Non-synonymous codons and termination codons (dependent on genetic code) are excluded. CAI values range from 0 to 1, with higher values indicating a higher proportion of the most abundant codons (see Sharp and Li, 1987, Nucleic Acids Research 15: 1281-1295; also see: Kim et al., Gene. 1997, 199:293-301; zur Megede et al., Journal of Virology, 2000, 74: 2628-2635). In certain embodiments, a nucleic acid molecule encoding a C1EI has a CAI of at least 0.75, 0.80, 0.85, 0.90, 0.95, or 0.99

The nucleotide sequence of SEQ ID NOS: 10-12 are codon optimized human C1EI nucleic acid sequences which were based on the sequence of the wild-type human C1EI nucleotide sequence (SEQ ID NO: 1).

Codon optimization can be performed, for example, using the DNA2.0 codon optimization algorithm, see Villalobos et al., “Gene Designer: a synthetic biology tool for constructing artificial DNA segments,” BMC Bioinformatics, vol. 7, article no: 285 (2006) or Operon/Eurofins Genomics codon optimization software.

For example, the nucleotide sequence can be modified to better reflect host codon usage to increase gene expression (e.g., protein production) in the host (e.g., a mammal). As another non-limiting example for the modification, one or more of the splice donors and/or splice acceptors in the nucleotide sequence of the protein of interest is modified to reduce the potential for extraneous splicing. As another non-limiting example for the modification, one or more introns can be inserted within or adjacent to the nucleotide sequence of the protein of interest to optimize AAV vector packaging and enhance expression.

In another embodiment, the nucleotide sequence coding for protein of interest has an improved codon usage bias for the human cell as compared to naturally occurring nucleotide sequence coding for the corresponding non-codon optimized sequence. The adaptiveness of a nucleotide sequence encoding a protein of interest to the codon usage of human cells may be expressed as codon adaptation index (CAI). A codon adaptation index is herein defined as a measurement of the relative adaptiveness of the codon usage of a gene towards the codon usage of highly expressed human genes. The relative adaptiveness (w) of each codon is the ratio of the usage of each codon, to that of the most abundant codon for the same amino acid. The CAI is defined as the geometric mean of these relative adaptiveness values. Non-synonymous codons and termination codons (dependent on genetic code) are excluded. CAI values range from 0 to 1, with higher values indicating a higher proportion of the most abundant codons (see Sharp and Li, 1987, Nucleic Acids Research 15: 1281-1295; also see: Kim et al., Gene. 1997, 199:293-301; zur Megede et al., Journal of Virology, 2000, 74: 2628-2635). In certain embodiments, a nucleic acid molecule encoding a protein of interest has a CAI of at least 0.75, 0.80, 0.85, 0.90, 0.95, or 0.99.

This can be done in conjunction with manually reducing CpG di-nucleotide content and removing any extra ORF in the sense and anti-sense direction. CpG di-nucleotide content has been shown to activate TLR9 in dendritic cells leading to potential immune activation and CTL responses. Our product in the AAV-vector genome delivered is ssDNA, thus reducing the CpG content, which may reduce liver inflammation and ALT.

Generally, codon optimization does not change the amino acid for which each codon encodes. It simply changes the nucleotide sequence so that it is more likely to be expressed at a relatively high level compared to the non-codon optimized sequence. This means that the nucleotide sequences of the nucleic acid provided herein and, for example, SEQ ID NO: 1 or 10-12 may be different but when they are translated the amino acid sequence of the protein that is produced is the same.

In some embodiments, the codon optimized hC1EI nucleic acid molecule has a CpG di-nucleotide content of less than 25, less than 20, less than 15, or less than 10. In another embodiment, the codon optimized hC1EI nucleic acid molecule has a GC content of less than 65%, less than 60%, or less than 58%.

It would be well within the capabilities of a skilled person to produce a nucleic acid molecule provided herein. This could be done, for example, using chemical synthesis of a given sequence. Further, suitable methods would be apparent to those skilled in the art for determining whether a nucleic acid described herein expresses a functional protein. For example, one suitable in vitro method involves inserting the nucleic acid into a vector, such as an AAV vector, transducing host cells, such as 293T or HeLa cells, with the vector, and assaying for C1EI activity. Alternatively, a suitable in vivo method involves transducing a vector containing the nucleic acid into HAE mice and assaying for functional C1EI in the plasma of the mice. Suitable methods are described in more detail below.

In some embodiments, the vector comprises one or more introns. The introns may facilitate processing of the RNA transcript in mammalian host cells, increase expression of the protein of interest and/or optimize packaging of the vector into AAV particles. Non-limiting examples of such an intron are a hemoglobin (β-globin) intron, hAAT intron and/or A1AT intron. In some embodiments, the intron is a synthetic intron. For example, the synthetic intron can include a nucleotide sequence having at least about 80%, 85%, 90%, at least about 95%, at least about 96%, at least about 97%, at least about 98%, at least about 99%, or more, sequence identity to SEQ ID NO: 6. The location and size of the intron in the vector can vary. In some embodiments, the intron is located between the promoter and the sequence encoding the protein of interest. In some embodiments, the intron is located downstream of the sequence encoding the protein of interest. In some embodiments, the intron is located within the promoter. In some embodiments, the intron includes an enhancer element. In some embodiments, the intron is located within the sequence encoding the protein of interest, preferably between exons of the sequence encoding the protein of interest. In some embodiments, the intron may comprise all or a portion of a naturally occurring intron within the sequence encoding the protein of interest. In some embodiments, the intron is a C1EI intron, for example, the second C1EI intron. In other embodiments, the intronic sequence is a composite hAAT/hemoglobin intron. In some embodiments, the intron also enhances expression of the C1EI-encoding nucleic acid.

In some embodiments, the vector construct may further comprise an exon sequence or fragment thereof, preferably adjacent to an intron sequence, e.g. an hAAT intron adjacent to an hAAT exon (SEQ ID NO: 72) or fragment thereof and/or a hemoglobin intron (SEQ ID NO: 70) adj acent to a hemoglobin exon (SEQ ID NO: 71) or fragment thereof.

In one or more embodiments, the intron comprises a nucleotide sequence at least 80% or 85% or 90% or 95% identical to SEQ ID NO: 61, and the intron may be about 100 to about 300 nucleotides in length, or about 150 to about 250 nucleotides in length. In example embodiments, the intron comprises a nucleotide sequence at least 80% or 85% or 90% or 95% identical to SEQ ID NO: 61 and is about 50-300 nucleotides, about 100-250 nucleotides, about 100-225 nucleotides, about 100-200 nucleotides, about 150-225 nucleotides, about 150-200 nucleotides, about 175-300 nucleotides, about 175-250 nucleotides, or about 150-250 nucleotides in length.

In some embodiments, the intron comprises SEQ ID NO: 67 or a fragment thereof. In one or more embodiments, the intron comprises a nucleotide sequence at least 80% or 85% or 90% or 95% identical to SEQ ID NO: 64, and the intron may be about 300 to about 600 nucleotides in length, or about 400 to about 500 nucleotides in length. In example embodiments, the intron comprises SEQ ID NO: 64 or a fragment thereof and is about 100-900 nucleotides, about 200-800 nucleotides, about 200-700 nucleotides, about 200-600 nucleotides, about 200-500 nucleotides, about 300-700 nucleotides, about 300-600 nucleotides, about 300-500 nucleotides, about 400-700 nucleotides, about 400-600 nucleotides, or about 400-500 nucleotides in length.

In one or more embodiments, the intron comprises a nucleotide sequence at least 80% or 85% or 90% or 95% identical to SEQ ID NO: 62, and the intron may be about 200 to about 500 nucleotides in length, or about 300 to about 400 nucleotides in length.

In one or more embodiments, the intron comprises a nucleotide sequence at least 80% or 85% or 90% or 95% identical to SEQ ID NO: 63, and the intron may be about 200 to about 500 nucleotides in length, or about 300 to about 400 nucleotides in length.

In one or more embodiments, the intron comprises a nucleotide sequence at least 80% or 85% or 90% or 95% identical to SEQ ID NO: 65, and the intron may be about 600 to about 1000 nucleotides in length, or about 800 to about 900 nucleotides in length.

In one or more embodiments, the intron comprises a nucleotide sequence at least 80% or 85% or 90% or 95% identical to SEQ ID NO: 66, and the intron may be about 1000 to about 2000 nucleotides in length, or about 1300 to about 1500 nucleotides in length.

In one or more embodiments, the intron comprises a nucleotide sequence at least 80% or 85% or 90% or 95% identical to SEQ ID NO: 67, and the intron may be about 1500 to about 2000 nucleotides in length, or about 1800 to about 1900 nucleotides in length.

In one or more embodiments, the intron comprises a nucleotide sequence at least 80% or 85% or 90% or 95% identical to SEQ ID NO: 68, and the intron may be about 50 to about 150 nucleotides in length.

In one or more embodiments, the intron comprises a nucleotide sequence at least 80% or 85% or 90% or 95% identical to SEQ ID NO: 69, and the intron may be about 50 to about 125 nucleotides in length.

In some embodiments, the vector constructs may further comprise an exon sequence or fragment thereof, preferably adjacent to an intron sequence. In an example embodiment, the vector construct comprises an hAAT intron adjacent to an exon comprising a nucleotide sequence at least 80% or 85% or 90% or 95% identical to SEQ ID NO: 72. In a further example embodiment, the vector construct comprises a hemoglobin intron adjacent to an exon sequence comprising a nucleotide sequence at least 80% or 85% or 90% or 95% identical to SEQ ID NO: 70. In an example embodiment, the vector comprises both (a) an hAAT intron adjacent to an exon comprising a nucleotide sequence at least 80% or 85% or 90% or 95% identical to SEQ ID NO: 72 and (b) a hemoglobin intron adjacent to an exon sequence comprising a nucleotide sequence at least 80% or 85% or 90% identical to SEQ ID NO: 70. In an example embodiment, the vector construct comprises an hAAT intron and a hemoglobin intron adj acent to a hemoglobin exon sequence comprising a nucleotide sequence at least 80% or 85% or 90% identical to SEQ ID NO: 71.

Inclusion of an intron element may enhance expression compared with expression in the absence of the intron element (see e.g. Kurachi et al., 1995, J Biol Chem. 1995 Mar 10; 270(10):5276-81). AAV vectors typically accept inserts of DNA having a defined size range which is generally about 4 kb to about 5.2 kb, or slightly more. However, there is no minimum size for packaging and small vector genomes package very efficiently. Introns and intron fragments fulfill this requirement while also enhancing expression. Thus, the present disclosure is not limited to the inclusion of C1EI intron sequences in the AAV vector, and include other introns or other DNA sequences in place of portions of a C1EI intron. Additionally, other 5′ and 3′ untranslated regions of nucleic acid may be used in place of those recited for human C1EI.

Polynucleotides and polypeptides including modified forms can be made using various standard cloning, recombinant DNA technology, via cell expression or in vitro translation and chemical synthesis techniques known to those of skill in the art (Sambrook et al., Molecular Cloning: A Laboratory Manual, 2nd edition).

### Methods of Gene Delivery

Also provided is a method of using vector construct or AAV particle as described herein to deliver a gene encoding the protein of interest. In one embodiment, a gene delivery vector may be a viral gene delivery vector, such as a viral particle, or a non-viral gene delivery vector, such as a vector construct or nucleic acid encoding the protein of interest. Viral vectors include lenti-, adeno-, herpes viral vectors. It is preferably a recombinant adeno-associated viral (rAAV) vector. Alternatively, non-viral systems may be used, including using naked DNA (with or without chromatin attachment regions) or conjugated DNA that is introduced into cells by various transfection methods such as lipids or electroporation.

A non-limiting example of a viral vector construct as described herein is provided in SEQ ID NO: 9, and includes an ApoE/HCR-hAAT promoter, hAAT/hemoglobin intron (hhI), wild-type coding sequence for human C1EI, and human growth hormone (hGH) poly(A) sequence (“HAE15” or “ApoE/HCR-hAAT.hhI.SERPIN G1.hGH”). Other non-limiting examples of a viral vector construct as described herein are provided in any one of SEQ ID NOs: 20-36, 57 and 58. Another vector construct comprising a promoter derived from the chicken β-actin (CBA) promoter sequence, a wild-type coding sequence for human C1EI, and a bovine growth factor (bGH) poly(A) sequence is set forth in SEQ ID NO: 8 (“CBA-HAE” or “CBA.SERPIN G1.bGH”).

In some embodiments, the vector construct or AAV vector genome comprises a nucleotide sequence having at least about 80%, 85%, 90%, at least about 95%, at least about 96%, at least about 97%, at least about 98%, at least about 99%, or more, sequence identity to SEQ ID NO: 9. In some embodiments, the vector construct or AAV vector genome comprises a nucleotide sequence having at least about 80%, 85%, 90%, at least about 95%, at least about 96%, at least about 97%, at least about 98%, at least about 99%, or more, sequence identity to any one of SEQ ID NO: 20-36. In some embodiments, the vector construct or AAV vector genome comprises a nucleotide sequence having at least about 80%, 85%, 90%, at least about 95%, at least about 96%, at least about 97%, at least about 98%, at least about 99%, or more, sequence identity to SEQ ID NO: 57. In some embodiments, the vector construct or AAV vector genome comprises a nucleotide sequence having at least about 80%, 85%, 90%, at least about 95%, at least about 96%, at least about 97%, at least about 98%, at least about 99%, or more, sequence identity to SEQ ID NO: 58.

The present disclosure finds use in both veterinary and medical applications. Suitable subjects for gene delivery methods as described herein include both avians and mammals, with mammals being preferred and humans being most preferred. Human subjects include neonates, infants, juveniles, and adults.

### Non-Viral Gene Delivery

Non-viral gene delivery may be carried out using naked DNA which is the simplest method of non-viral transfection. It may be possible, for example, to administer the vector constructs provided herein using naked plasmid DNA. Alternatively, methods such as electroporation, sonoporation or the use of a “gene gun”, which shoots DNA coated gold particles into the cell using, for example, high pressure gas or an inverted .22 calibre gun, may be used (Helios® Gene Gun System (BIO-RAD)).

To improve the delivery of a vector construct into a cell, it may be necessary to protect it from damage and its entry into the cell may be facilitated. To this end, lipoplexes and polyplexes may be used that have the ability to protect a nucleic acid from undesirable degradation during the transfection process.

Vector constructs may be coated with lipids in an organized structure such as a micelle or a liposome. When the organized structure is complexed with DNA it is called a lipoplex. Anionic and neutral lipids may be used for the construction of lipoplexes for synthetic vectors. In one embodiment, cationic lipids, due to their positive charge, may be used to condense negatively charged DNA molecules so as to facilitate the encapsulation of DNA into liposomes. If may be necessary to add helper lipids (usually electroneutral lipids, such as DOPE) to cationic lipids so as to form lipoplexes (Dabkowska et al., J R Soc Interface. 2012 Mar 7; 9(68): 548-561).

In certain embodiments, complexes of polymers with DNA, called polyplexes, may be used to deliver a vector construct. Most polyplexes consist of cationic polymers and their production is regulated by ionic interactions. Polyplexes typically cannot release their DNA load into the cytoplasm. Thus, co-transfection with endosome-lytic agents (to lyse the endosome that is made during endocytosis, the process by which the polyplex enters the cell), such as inactivated adenovirus, may be necessary (Akinc et al., The Journal of Gene Medicine. 7 (5): 657-63).

In certain embodiments, hybrid methods may be used to deliver a vector construct that combines two or more techniques. Virosomes are one example; they combine liposomes with an inactivated HIV or influenza virus. In another embodiment, other methods involve mixing other viral vectors with cationic lipids or hybridizing viruses and may be used to deliver a nucleic acid (Khan, Firdos Alam, Biotechnology Fundamentals, CRC Press, Nov. 18, 2015, p. 395).

In certain embodiments, a dendrimer may be used to deliver a vector construct, in particular, a cationic dendrimer, i.e. one with a positive surface charge. When in the presence of genetic material as DNA or RNA, charge complementarity leads to a temporary association of the nucleic acid with the cationic dendrimer. On reaching its destination the dendrimer-nucleic acid complex is then imported into the cell via endocytosis (Amiji, Mansoor M. ed., Polymeric Gene Delivery: Principles and Applications, CRC Press, Sep. 29, 2004, p. 142.)

### Viral Particles

In one embodiment, a suitable viral gene delivery vector such as a viral particle may be used to deliver a nucleic acid. In certain embodiments, viral gene delivery vectors suitable for use herein may be a parvovirus, an adenovirus, a retrovirus, a lentivirus or a herpes simplex virus. The parvovirus may be an adenovirus-associated virus (AAV).

Accordingly, the present disclosure provides viral particles for use as gene delivery vectors (comprising a vector construct provided herein) based on animal parvoviruses, in particular dependoviruses such as infectious human or simian AAV, and the components thereof (e.g., an animal parvovirus genome) for introduction and/or expression of a C1EI in a mammalian cell. The term “parvoviral” as used herein thus encompasses dependoviruses such as any type of AAV.

Viruses of the Parvoviridae family are small DNA animal viruses. The family Parvoviridae may be divided between two subfamilies: the Parvovirinae, which infect vertebrates, and the Densovirinae, which infect insects. Members of the subfamily Parvovirinae are herein referred to as the parvoviruses and include the genus Dependovirus. As may be deduced from the name of their genus, members of the Dependovirus are unique in that they usually require coinfection with a helper virus such as adenovirus or herpes virus for productive infection in cell culture. The genus Dependovirus includes AAV, which normally infects humans (e.g., serotypes 1, 2, 3A, 3B, 4, 5, and 6), primates (e.g., serotypes 1 and 4), and related viruses that infect other warm-blooded animals (e.g., bovine, canine, equine, mice, rats, and ovine adeno-associated viruses) in addition to birds and reptiles. Further information on parvoviruses and other members of the Parvoviridae is described in Kenneth I. Berns, “Parvoviridae: The Viruses and Their Replication,” Chapter 69 in Fields Virology (3d Ed. 1996). For convenience the present disclosure is further exemplified and described herein by reference to AAV. It is, however, understood that the present disclosure is not limited to AAV but may equally be applied to other parvoviruses.

Production of AAV particles requires AAV “rep” and “cap” genes, which are genes encoding replication and encapsidation proteins, respectively. AAV rep and cap genes have been found in all AAV serotypes examined to date, and are described herein and in the references cited. In wild-type AAV, the rep and cap genes are generally found adjacent to each other in the viral genome (i.e., they are “coupled” together as adjoining or overlapping transcriptional units), and they are generally conserved among AAV serotypes. AAV rep and cap genes are also individually and collectively referred to as “AAV packaging genes.” The AAV cap genes for use herein encode Cap proteins which are capable of packaging AAV vectors in the presence of rep and adeno helper function and are capable of binding target cellular receptors. In some embodiments, the AAV cap gene encodes a capsid protein having an amino acid sequence derived from a particular AAV serotype.

The AAV sequences employed for the production of AAV can be derived from the genome of any AAV serotype. Generally, the AAV serotypes have genomic sequences of significant homology at the amino acid and the nucleic acid levels, provide a similar set of genetic functions, produce virions which are essentially physically and functionally equivalent, and replicate and assemble by practically identical mechanisms. For the genomic sequence of AAV serotypes and a discussion of the genomic similarities. (See, e.g., GenBank Accession number U89790; GenBank Accession number J01901; GenBank Accession number AF043303; GenBank Accession number AF085716; Chiorini et al., J. Vir. (1997) vol. 71, pp. 6823-6833; Srivastava et al., J. Vir. (1983) vol. 45, pp. 555-564; Chiorini et al., J. Vir. (1999) vol. 73, pp. 1309-1319; Rutledge et al., J. Vir. (1998) vol. 72, pp. 309-319; and Wu et al., J. Vir. (2000) vol. 74, pp. 8635-8647).

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins form the capsid. The assembly-activating protein (AAP) rapidly chaperones capsid assembly and prevents degradation of free capsid proteins (Grosse et al., J. Virol. 91(20):e01198-17, 2017). The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. The Rep genes encode the Rep proteins, Rep78, Rep68, Rep52, and Rep40. Rep78 and Rep68 are transcribed from the p5 promoter, and Rep 52 and Rep40 are transcribed from the p19 promoter. The cap genes encode the VP proteins, VP1, VP2, and VP3. The cap genes are transcribed from the p40 promoter. The ITRs employed in the vectors of the present embodiment may correspond to the same serotype as the associated cap genes, or may differ. In one embodiment, the ITRs employed herein correspond to an AAV2 serotype and the cap genes correspond to an AAV5 serotype.

The AAV VP proteins are known to determine the cellular tropicity of the AAV virion. The VP protein-encoding sequences are significantly less conserved than Rep proteins and genes among different AAV serotypes. The ability of Rep and ITR sequences to cross-complement corresponding sequences of other serotypes allows for the production of pseudotyped AAV particles comprising the capsid proteins of a serotype (e.g., AAV1, 5 or 8) and the Rep and/or ITR sequences of another AAV serotype (e.g., AAV2). Such pseudotyped rAAV particles are a part of the present disclosure.

The AAV particles described herein (and the encoding AAV vector genomes) may comprise any of the capsid proteins described in WO 2018/022608 or PCT/US 19/32097, incorporated by reference herein in its entirety for its disclosure of human and simian AAV capsids and their properties such as transduction efficiency, tissue tropism, glycan-binding, and resistance to neutralization by IVIG, including but not limited to any of the capsids in the sequence listing and variants thereof, e.g. with chimeric swapped variable regions and/or glycan binding sequences and/or GH loop.

In one embodiment, the AAV ITR sequences for use in the context of the present disclosure are derived from AAV1, AAV2, AAV4 and/or AAV6. Likewise, the Rep (e.g., Rep78 and Rep52) coding sequences are in one embodiment derived from AAV1, AAV2, AAV4 and/or AAV6. The sequences coding for the VP1, VP2, and VP3 capsid proteins for use in the context of the present disclosure may however be taken from any serotype, such as from AAV1, AAV2, AAV3, AAV4, AAV5, AAV6, AAV7, AAV8, AAV9, AAV10, AAV11 or AAV 12, or from simian AAVs, including any of the capsid proteins described in WO 2018/022608 or PCT/US19/32097, or newly developed AAV-like particles obtained by e.g. capsid shuffling techniques and AAV capsid libraries, or any capsid at least 90% identical to any of SEQ ID NO: 37-53 or 56.

For example, the amino acid sequences of various capsids are published. See, e.g.,
AAVRh.1 / hu.14 / AAV9 AAS99264.1 (SEQ ID NO: 37)
AAVRh.8 SEQ97 of U.S. Pat. Pub. 2013/0045186 (SEQ ID NO: 38)
AAVRh.10 SEQ81 of U.S. Pat. Pub. 2013/0045186 (SEQ ID NO: 39)
AAVRh.74 SEQ 1 of Int′l. Pat. Pub. WO 2013/123503(SEQ ID NO: 40)
AAV1 AAB_95452.1 (SEQ ID NO: 41)
AAV2 YP_680426.1 (SEQ ID NO: 42)
AAV3 NP_043941.1 (SEQ ID NO: 43)
AAV3B AAB95452.1 (SEQ ID NO: 44)
AAV4 NP_044927.1 (SEQ ID NO: 45)
AAV5 YP_068409.1 (SEQ ID NO: 46)
AAV6 AAB95450.1 (SEQ ID NO: 47)
AAV7 YP_077178.1 (SEQ ID NO: 48)
AAV8 YP_077180.1 (SEQ ID NO: 49)
AAV10 AAT46337.1 (SEQ ID NO: 50)
AAV11 AAT46339.1 (SEQ ID NO: 51)
AAV12 ABI16639.1 (SEQ ID NO: 52)
AAV13 ABZ10812.1 (SEQ ID NO: 53)

Modified “AAV” sequences also can be used in the context of the present disclosure, e.g. for the production of AAV gene therapy vectors. Such modified sequences e.g. sequences having at least about 70%, at least about 75%, at least about 80%, at least about 85%, at least about 90%, at least about 95%, or more nucleotide and/or amino acid sequence identity (e.g., a sequence having about 75-99% nucleotide sequence identity) to an AAV1, AAV2, AAV3, AAV4, AAV5, AAV6, AAV7, AAV8 or AAV9 ITR, Rep, or VP, can be used in place of wild-type AAV ITR, Rep, or VP sequences.

In some embodiments, a nucleic acid sequence encoding an AAV capsid protein is operably linked to expression control sequences for expression in a specific cell type, such as Sf9 or HEK cells. Techniques known to one skilled in the art for expressing foreign genes in insect host cells or mammalian host cells can be used to practice the embodiment. Methodology for molecular engineering and expression of polypeptides in insect cells is described, for example, in Summers and Smith (1986) A Manual of Methods for Baculovirus Vectors and Insect Culture Procedures, Texas Agricultural Experimental Station Bull. No. 7555, College Station, Tex.; Luckow (1991) In Prokop et al., Cloning and Expression of Heterologous Genes in Insect Cells with Baculovirus Vectors’ Recombinant DNA Technology and Applications, 97-152; King, L. A. and R. D. Possee (1992) The baculovirus expression system, Chapman and Hall, United Kingdom; O’Reilly, D. R., L. K. Miller, V. A. Luckow (1992) Baculovirus Expression Vectors: A Laboratory Manual, New York; W.H. Freeman and Richardson, C. D. (1995) Baculovirus Expression Protocols, Methods in Molecular Biology, volume 39; U.S. Pat. No. 4,745,051; US2003148506; and WO 03/074714, all of which are incorporated by reference in their entireties. A particularly suitable promoter for transcription of a nucleotide sequence encoding an AAV capsid protein is e.g. the polyhedron promoter. However, other promoters that are active in insect cells are known in the art, e.g. the p10, p35 or IE-1 promoters and further promoters described in the above references are also contemplated.

Use of insect cells for expression of heterologous proteins is well documented, as are methods of introducing nucleic acids, such as vectors, e.g., insect-cell compatible vectors, into such cells and methods of maintaining such cells in culture. (See, e.g., METHODS IN MOLECULAR BIOLOGY, ed. Richard, Humana Press, N J (1995); O’Reilly et al., BACULOVIRUS EXPRESSION VECTORS, A LABORATORY MANUAL, Oxford Univ. Press (1994); Samulski et al., J. Vir. (1989) vol. 63, pp.3822-3828; Kajigaya et al., Proc. Nat′l. Acad. Sci. USA (1991) vol. 88, pp. 4646-4650; Ruffing et al., J. Vir. (1992) vol. 66, pp. 6922-6930; Kirnbauer et al., Vir. (1996) vol. 219, pp. 37-44; Zhao et al., Vir. (2000) vol. 272, pp. 382-393; and U.S. Pat. No. 6,204,059). In some embodiments, the nucleic acid construct encoding AAV in insect cells is an insect cell-compatible vector. An “insect cell-compatible vector” or “vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cells genome but the presence of the vector in the insect cell need not be permanent and transient episomal vectors are also included. The vectors can be introduced by any means known, for example by chemical treatment of the cells, electroporation, or infection. In some embodiments, the vector is a baculovirus, a viral vector, or a plasmid. In one embodiment, the vector is a baculovirus, i.e. the construct is a baculoviral vector. Baculoviral vectors and methods for their use are described in the above cited references on molecular engineering of insect cells.

### Methods for Producing Recombinant AAV Particles

The present disclosure provides materials and methods for producing recombinant AAV particles in insect or mammalian cells that comprise any of the vector constructs described herein. In some embodiments, the vector construct further comprises a promoter and a restriction site downstream of the promoter to allow insertion of a polynucleotide encoding one or more proteins of interest, wherein the promoter and the restriction site are located downstream of the 5′ AAV ITR and upstream of the 3′ AAV ITR. In some embodiments, the vector construct further comprises a posttranscriptional regulatory element downstream of the restriction site and upstream of the 3′ AAV ITR. In some embodiments, the vector construct further comprises a polynucleotide inserted at the restriction site and operably linked with the promoter, where the polynucleotide comprises the coding region of a protein of interest. As a skilled artisan will appreciate, any one of the AAV vector constructs disclosed in the present application can be used in methods to produce the recombinant AAV particle.

In some embodiments, the helper functions are provided by one or more helper plasmids or helper viruses comprising adenoviral or baculoviral helper genes. Non-limiting examples of the adenoviral or baculoviral helper genes include, but are not limited to, E1A, E1B, E2A, E4 and VA, which can provide helper functions to AAV packaging.

Helper viruses of AAV are known in the art and include, for example, viruses from the family Adenoviridae and the family Herpes viridae. Examples of helper viruses of AAV include, but are not limited to, SAdV-13 helper virus and SAdV-13-like helper virus described in U.S. Publication No. 20110201088 (the disclosure of which is incorporated herein by reference), and helper vectors pHELP (Applied Viromics). A skilled artisan will appreciate that any helper virus or helper plasmid of AAV that can provide adequate helper function to AAV can be used herein.

In some embodiments, the AAV cap genes are present in a plasmid. The plasmid can further comprise an AAV rep gene which may or may not correspond to the same serotype as the cap genes. The cap genes and/or rep gene from any AAV serotype described herein (including, but not limited to, AAV1, AAV2, AAV4, AAV5, AAV6, AAV7, AAV8, AAV9, AAV10, AAV11, AAV12, AAV13 and any variants thereof) can be used to produce the recombinant AAV. In some embodiments, the AAV cap genes encode a capsid from serotype 1, serotype 2, serotype 4, serotype 5, serotype 6, serotype 7, serotype 8, serotype 9, serotype 10, serotype 11, serotype 12, serotype 13 or a variant thereof.

In some embodiments, the insect or mammalian cell can be transfected with the helper plasmid or helper virus, the vector construct and the plasmid encoding the AAV cap genes; and the recombinant AAV virus can be collected at various time points after co-transfection. For example, the recombinant AAV virus can be collected at about 12 hours, about 24 hours, about 36 hours, about 48 hours, about 72 hours, about 96 hours, about 120 hours, or a time between any of these two time points after the co-transfection.

Recombinant AAV particles can also be produced using any conventional methods known in the art suitable for producing infectious recombinant AAV. In some instances, a recombinant AAV can be produced by using an insect or mammalian cell that stably expresses some of the necessary components for AAV particle production. For example, a plasmid (or multiple plasmids) comprising AAV rep and cap genes, and a selectable marker, such as a neomycin resistance gene, can be integrated into the genome of the cell. The insect or mammalian cell can then be co-infected with a helper virus (e.g., adenovirus or baculovirus providing the helper functions) and the viral vector construct comprising the 5′ and 3′ AAV ITR (and the nucleotide sequence encoding the heterologous protein, if desired). The advantages of this method are that the cells are selectable and are suitable for large-scale production of the recombinant AAV particle. As another non-limiting example, adenovirus or baculovirus rather than plasmids can be used to introduce rep and cap genes into packaging cells. As yet another non-limiting example, both the viral vector construct containing the 5′ and 3′ AAV ITRs and the rep-cap genes can be stably integrated into the DNA of producer cells, and the helper functions can be provided by a wild-type adenovirus to produce the recombinant AAV.

In one aspect, provided herein are methods for the production of a AAV particle, useful as a gene delivery vector, the method comprising the steps of:
(a) providing a cell permissive for AAV replication (e.g. an insect cell or a mammalian cell) with one or more nucleic acid constructs comprising:
   (i) a nucleic acid molecule (e.g. recombinant vector construct) provided herein that is flanked by at least one AAV inverted terminal repeat nucleotide sequence;
   (ii) a nucleotide sequence encoding one or more AAV Rep proteins which is operably linked to a promoter that is capable of driving expression of the Rep protein(s) in the cell;
   (iii) a nucleotide sequence encoding one or more AAV capsid proteins which is operably linked to a promoter that is capable of driving expression of the capsid protein(s) in the cell;
   (iv) and optionally AAP and MAAP contained in the VP⅔ mRNA
(b) culturing the cell defined in (a) under conditions conducive to the expression of the Rep and the capsid proteins; and,
optionally, (c) recovering the AAV gene delivery vector, and
optionally (d) purifying the AAV particle. For example, the recombinant vector construct of (i) comprises (1) at least one AAV ITR, (2) a heterologous liver-specific transcription regulatory region as described herein, and (3) a nucleic acid encoding a functional C1EI. Preferably the recombinant vector construct of (i) comprises both a 5′ and 3′ AAV ITR.

Typically then, a method provided herein for producing a AAV gene delivery vector comprises: providing to a cell permissive for AAV replication (a) a nucleotide sequence encoding a template for producing vector genome, e.g. vector construct of the present disclosure (as described in detail herein); (b) nucleotide sequences sufficient for replication of the template to produce a vector genome (the first expression cassette defined above); (c) nucleotide sequences sufficient to package the vector genome into an AAV capsid (the second expression cassette defined above), under conditions sufficient for replication and packaging of the vector genome into the AAV capsid, whereby AAV particles comprising the vector genome encapsidated within the AAV capsid are produced in the cell.

Transient transfection of adherent HEK293 cells (Chahal et al., J. Virol. Meth. 196: 163-73 (2014)) and transfection of Sf9 cells, using the baculovirus expression vector system (BEVS) (Mietzsch et al., Hum. Gene Ther. 25: 212-22 (2014)), are two of the most commonly used methods to produce AAV vectors.

A method provided herein may comprise the step of affinity-purification of the (virions comprising the) recombinant parvoviral (rAAV) vector using an anti-AAV antibody, in one embodiment an immobilized antibody. In another embodiment,the anti-AAV antibody is a monoclonal antibody. One antibody for use herein is a single chain camelid antibody or a fragment thereof as e.g. obtainable from camels or llamas (see e.g. Muyldermans, 2001, Biotechnol. 74: 277-302). The antibody for affinity-purification of rAAV is an antibody that specifically binds an epitope on an AAV capsid protein, whereby in one embodiment the epitope is an epitope that is present on capsid protein of more than one AAV serotype. For example, the antibody may be raised or selected on the basis of specific binding to AAV5 capsid but at the same time also it may also specifically bind to AAV1, AAV2, AAV3, AAV6, AAV8 or AAV9 capsids

The methods provided herein for producing rAAV particles produce a population of rAAV particles. In some embodiments, the population is enriched for particles comprising full length or nearly full-length vector genomes by steps that reduce the number of empty capsids.

The population of rAAV particles produced by the methods provided herein are used, for example, for administration in any of the treatment methods described herein.

### Cell Types Used in AAV Particle Production

The viral particles comprising the vector constructs described herein may be produced using any invertebrate cell type which allows for production of AAV or biologic products and which can be maintained in culture. For example, the insect cell line used can be from Spodoptera frugiperda, such as SF9, SF21, SF900+, drosophila cell lines, mosquito cell lines, e.g., Aedes albopictus derived cell lines, domestic silkworm cell lines, e.g. Bombyx mori cell lines, Trichoplusia ni cell lines such as High Five cells or Lepidoptera cell lines such as Ascalapha odorata cell lines. In one embodiment, insect cells are cells from the insect species which are susceptible to baculovirus infection, including High Five, Sf9, Se301, SeIZD2109, SeUCR1, Sf9, Sf900+, Sf21, BTI-TN-5B1-4, MG-1, Tn368, HzAm1, BM-N, Ha2302, Hz2E5 and Ao38.

Baculoviruses are enveloped DNA viruses of arthropods, two members of which are well known expression vectors for producing recombinant proteins in cell cultures. Baculoviruses have circular double-stranded genomes (80-200 kbp) which can be engineered to allow the delivery of large genomic content to specific cells. The viruses used as a vector are generally Autographa californica multicapsid nucleopolyhedrovirus (AcMNPV) or Bombyx mori nucleopolyhedrovirus (BmNPV) (Kato et al., (2010), Applied Microbiology and Biotechnology, vol. 85, Issue 3, pp 459-470).

Baculoviruses are commonly used for the infection of insect cells for the expression of recombinant proteins. In particular, expression of heterologous genes in insects can be accomplished as described in for instance U.S. Pat. No. 4,745,051; EP 127,839; EP 155,476; Vlak et al., (1988), Journal of General Virology, vol. 68, pp 765-776; Miller et al., (1988), Annual Review of Microbiology, vol. 42, pp 177-179; Carbonell et al., (1998), Gene, vol. 73, Issue 2, pp 409-418; Maeda et al., (1985), Nature, vol. 315, pp 592-594; Lebacq-Veheyden et al., (1988), Molecular and Cellular Biology, vol. 8, no. 8, pp 3129-3135; Smith et al., (1985), PNAS, vol. 82, pp 8404-8408; and Miyajima et al., (1987), Gene, vol. 58, pp 273-281. Numerous baculovirus strains and variants and corresponding permissive insect host cells that can be used for protein production are described in Luckow et al., (1988), Nature Biotechnology, vol. 6, pp 47-55; Maeda et al., (1985), Nature, vol. 315, pp 592-594; and McKenna et al., (1998), Journal of Invertebrate Pathology, vol. 71, Issue 1, pp 82-90.

In another embodiment, the methods provided herein are carried out with any mammalian cell type which allows for replication of AAV or production of biologic products, and which can be maintained in culture. In one embodiment, mammalian cells used can be HEK293, HeLa, CHO, NSO, SP2/0, PER.C6, Vero, RD, BHK, HT 1080, A549, Cos-7, ARPE-19, and MRC-5 cells.

### Host Organism and/or Cells

In a further embodiment, a host cell is provided comprising the vector described above. In one embodiment, the vector construct is capable of expressing the nucleic acid molecule provided herein in the host cell. In some embodiments, provided herein are HAE therapeutics that are host cells comprising a vector construct comprising a nucleic acid encoding hC1EI, for use in HAE cell therapy.

As used herein, the term “host” refers to organisms and/or cells which harbour a nucleic acid molecule or a vector construct of the present disclosure, as well as organisms and/or cells that are suitable for use in expressing a recombinant gene or protein. It is not intended that the present disclosure be limited to any particular type of cell or organism. Indeed, it is contemplated that any suitable organism and/or cell will find use herein as a host. A host cell may be in the form of a single cell, a population of similar or different cells, for example in the form of a culture (such as a liquid culture or a culture on a solid substrate), an organism or part thereof. In one embodiment, a host cell may permit the expression of a nucleic acid molecule provided herein. Thus, the host cell may be, for example, a bacterial, a yeast, an insect or a mammalian cell, or a human cell.

In another embodiment, provided is a means for delivering a nucleic acid provided herein into a broad range of cells, including dividing and non-dividing cells. The present disclosure may be employed to deliver a nucleic acid provided herein to a cell in vitro, e. g. to produce a polypeptide encoded by such a nucleic acid molecule in vitro or for ex vivo gene therapy.

The nucleic acid molecule, vector construct, cells and methods/use of the present disclosure are additionally useful in a method of delivering a nucleic acid provided here into a host, typically a host suffering from HAE.

### Pharmaceutical Formulations

In one embodiment, provided is a pharmaceutical composition comprising a nucleic acid or a vector provided herein and a pharmaceutically acceptable diluent, excipient, carrier and/or other medicinal agent, pharmaceutical agent or adjuvant, etc.

By “pharmaceutically acceptable” it is meant a material that is not biologically or otherwise undesirable, i.e., the material may be administered to a subject without causing any undesirable biological effects. Thus, such a pharmaceutical composition may be used, for example, in transfection of a cell ex vivo or in administering a viral particle or cell directly to a subject.

A carrier may be suitable for parenteral administration, which includes intravenous, intraperitoneal or intramuscular administration. Alternatively, the carrier may be suitable for sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions provided herein is contemplated.

In other embodiments, provided herein are pharmaceutical compositions (i.e. formulations) of AAV particles useful for administration to subjects suffering from a genetic disorder to deliver gene encoding a protein of interest. In certain embodiments, the pharmaceutical formulations provided herein are liquid formulations that comprise recombinant AAV particles comprising any of the vector constructs disclosed herein. The concentration of recombinant AAV virions in the formulation may vary. In certain embodiments, the concentration of recombinant AAV particle in the formulation may range from 1E12 vg/ml to 5E14 vg/ml. In one embodiment, the concentration of recombinant AAV particle in the formulation is about 6E13 vg/ml.

In other embodiments, the AAV particle pharmaceutical formulation provided herein comprises one or more sterile pharmaceutically acceptable excipients to provide the formulation with advantageous properties for storage and/or administration to subjects for the treatment of the genetic disorder. In certain embodiments, the pharmaceutical formulations provided herein are capable of being stored at -65° C. for a period of at least 2 weeks, in one embodiment at least 4 weeks, in another embodiment at least 6 weeks and yet another embodiment at least about 8 weeks, without detectable change in stability. In this regard, the term “stable” means that the recombinant AAV particle present in the formulation essentially retains its physical stability, chemical stability and/or biological activity during storage. In certain embodiments, the recombinant AAV particle present in the pharmaceutical formulation retains at least about 80% of its biological activity in a human patient during storage for a determined period of time at -65° C., in other embodiments at least about 85%, 90%, 95%, 98% or 99% of its biological activity in a human subject. In one embodiment the subjects are juvenile human subjects.

In certain aspects, the formulation comprising recombinant AAV particle further comprises one or more buffering agents. For example, in various embodiments, the formulation provided herein comprises sodium phosphate dibasic at a concentration of about 0.1 mg/ml to about 3 mg/ml, about 0.5 mg/ml to about 2.5 mg/ml, about 1 mg/ml to about 2 mg/ml, or about 1.4 mg/ml to about 1.6 mg/ml. In one embodiment, the AAV particle formulation provided herein comprises about 1.42 mg/ml of sodium phosphate, dibasic (dried). Another buffering agent that may find use in the recombinant AAV particle formulations provided herein is sodium phosphate, monobasic monohydrate which, in some embodiments, finds use at a concentration of from about 0.1 mg/ml to about 3 mg/ml, about 0.5 mg/ml to about 2.5 mg/ml, about 1 mg/ml to about 2 mg/ml, or about 1.3 mg/ml to about 1.5 mg/ml. In one embodiment, the AAV particle formulation of the present embodiment comprises about 1.38 mg/ml of sodium phosphate, monobasic monohydrate. In another embodiment, the recombinant AAV particle formulation provided herein comprises about 1.42 mg/ml of sodium phosphate, dibasic and about 1.38 mg/ml of sodium phosphate, monobasic monohydrate.

In another embodiment, the recombinant AAV particle formulation provided herein may comprise one or more isotonicity agents, such as sodium chloride, in one embodiment at a concentration of about 1 mg/ml to about 20 mg/ml, for example, about 1 mg/ml to about 10 mg/ml, about 5 mg/ml to about 15 mg/ml, or about 8 mg/ml to about 20 mg/ml. In another embodiment, the recombinant AAV particle formulation provided herein comprises about 8.18 mg/ml sodium chloride. Other buffering agents and isotonicity agents known in the art are suitable and may be routinely employed for use in the formulations provided herein.

In another embodiment, the recombinant AAV particle formulations provided herein may comprise one or more bulking agents. Exemplary bulking agents include without limitation mannitol, sucrose, dextran, lactose, trehalose, and povidone (PVP K24). In certain embodiments, the formulations provided herein comprise mannitol, which may be present in an amount from about 5 mg/ml to about 40 mg/ml, or from about 10 mg/ml to about 30 mg/ml, or from about 15 mg/ml to about 25 mg/ml. In another embodiment, mannitol is present at a concentration of about 20 mg/ml.

In yet another embodiment, the recombinant AAV particle formulations provided herein may comprise one or more surfactants, which may be non-ionic surfactants. Exemplary surfactants include ionic surfactants, non-ionic surfactants, and combinations thereof. For example, the surfactant can be, without limitation, TWEEN 80 (also known as polysorbate 80, or its chemical name polyoxyethylene sorbitan monooleate), sodium dodecylsulfate, sodium stearate, ammonium lauryl sulfate, TRITON AG 98 (Rhone-Poulenc), poloxamer 407, poloxamer 188 and the like, and combinations thereof. In one embodiment, the formulation of the present embodiment comprises poloxamer 188, which may be present at a concentration of from about 0.1 mg/ml to about 4 mg/ml, or from about 0.5 mg/ml to about 3 mg/ml, from about 1 mg/ml to about 3 mg/ml, about 1.5 mg/ml to about 2.5 mg/ml, or from about 1.8 mg/ml to about 2.2 mg/ml. In another embodiment, poloxamer 188 is present at a concentration of about 2.0 mg/ml.

The recombinant AAV particle formulations provided herein are stable and can be stored for extended periods of time without an unacceptable change in quality, potency, or purity. In one aspect, the formulation is stable at a temperature of about 5° C. (e.g., 2° C. to 8° C.) for at least 1 month, for example, at least 1 month, at least 3 months, at least 6 months, at least 12 months, at least 18 months, at least 24 months, or more. In another embodiment, the formulation is stable at a temperature of less than or equal to about -20° C. for at least 6 months, for example, at least 6 months, at least 12 months, at least 18 months, at least 24 months, at least 36 months, or more. In another embodiment, the formulation is stable at a temperature of less than or equal to about -40° C. for at least 6 months, for example, at least 6 months, at least 12 months, at least 18 months, at least 24 months, at least 36 months, or more. In another embodiment, the formulation is stable at a temperature of less than or equal to about -60° C. for at least 6 months, for example, at least 6 months, at least 12 months, at least 18 months, at least 24 months, at least 36 months, or more.

Pharmaceutical compositions are typically sterile and stable under the conditions of manufacture and storage. Pharmaceutical compositions may be formulated as a solution, microemulsion, liposome, or other ordered structure suitable to accommodate high drug concentration. The carrier may be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In some embodiments, isotonic agents, for example, sugars, polyalcohols such as mannitol, sorbitol, or sodium chloride are included in the composition. Prolonged absorption of the injectable compositions can be brought about by including in the composition an agent which delays absorption, for example, monostearate salts and gelatin. In certain embodiments, a nucleic acid or vector construct provided herein may be administered in a time or controlled release formulation, for example in a composition which includes a slow release polymer or other carriers that will protect the compound against rapid release, including implants and microencapsulated delivery systems. Biodegradable, biocompatible polymers may for example be used, such as ethylene vinyl acetate, polyanhydrides, polyglycolic acid, collagen, polyorthoesters, polylactic acid and polylactic, polyglycolic copolymners (PLG).

In certain embodiments, the pharmaceutical composition comprising the vector construct or AAV particle provided herein may be of use in transferring genetic material to a cell. Such transfer may take place in vitro, ex vivo or in vivo. Accordingly, one embodiment provides a method for delivering a nucleotide sequence to a cell, which method comprises contacting a nucleic acid, a vector construct, or a pharmaceutical composition as described herein under conditions such the nucleic acid or vector provided herein enters the cell. The cell may be a cell in vitro, ex vivo or in vivo.

### Methods of Treatment

In certain embodiments, provided herein are methods for treating a subject suffering from a genetic disorder comprising administering to the subject a therapeutically effective amount of a nucleic acid encoding C1EI, a vector construct, an AAV particle, or a host cell expressing a C1EI, or a pharmaceutical composition comprising the same. In this instance, a “therapeutically effective amount” is an amount that after administration results in the expression of the therapeutic protein in a level sufficient to at least partially and preferably fully ameliorate the symptoms of the genetic disorder.

In one embodiment, provided herein is a method of treating C1EI deficiency comprising administering a therapeutically effective amount of a nucleic acid, a vector construct, an AAV particle, a host cell or a pharmaceutical composition provided herein to a patient suffering from a C1EI deficiency, for example HAE. In one embodiment, the patient is human. In one embodiment, the subject patient population is patients with moderate to severe C1EI deficiency, including those with HAE, or variant forms of HAE. In one embodiment, the goal for the treatment is conversion of severe HAE patients to either moderate or mild HAE thus lessening the burden associated with a recurrent acute HAE attacks. In one embodiment, the treatment increases functional C1EI levels in blood to normal range or at least 40% of the normal range of about 16 mg/dL (or 1 IU/ml) to about 32 mg/dL. In related embodiments, the treatment ameliorates HAE symptoms or reduces the frequency, duration or severity of acute HAE attacks. In some embodiments, the treatment reduces the amount of on-demand therapy (e.g. human C1EI protein, kallikrein inhibitor, bradykinin antagonist, etc.) required to treat acute HAE attacks, or reduces the frequency with which on-demand therapy is administered to treat acute HAE attacks. In some embodiments, subjects that received the treatment experience at least a 50%, 60%, 70%, 80% or 90% reduction in attack frequency compared to subjects that did not receive the treatment.

In one embodiment, provided herein are methods for increasing circulating C1EI protein levels in the blood of a subject in need thereof comprising administering to the subject any of the nucleic acids, vector constructs, AAV particles, host cells, or pharmaceutical compositions provided herein, that express the C1EI protein.

In another embodiment, provided herein is the use of an effective amount of recombinant AAV particle described herein for the preparation of a medicament for the treatment of a subject suffering from deficiency of functional C1EI or HAE. In one embodiment, the subject suffering from HAE is a human. In one embodiment, the medicament is administered by intravenous (IV) administration. In another embodiment, administration of the medicament results in expression of C1EI protein in the bloodstream of the subject sufficient to increase functional C1EI levels in blood in the subject to at least normal range or at least 40% of the normal range of about 16 mg/dL (or 1 IU/ml) to about 32 mg/dL.

In one or more embodiments, the treatment methods provided herein also comprise administration of a prophylactic and/or therapeutic corticosteroid for the prevention and/or treatment of any hepatotoxicity associated with administration of the AAV C1EI virus. The prophylactic or therapeutic corticosteroid treatment may comprise at least 5, 10, 15, 20, 25, 30, 35, 40, 45, 50, 55, 60, or more mg/day of the corticosteroid. In certain embodiments, the prophylactic or therapeutic corticosteroid may be administered over a continuous period of at least about 3, 4, 5, 6, 7, 8, 9, 10 weeks, or more.

In or more embodiments, the treatment methods provided herein optionally include administration, e.g. concurrent administration, of other therapies that are used to treat HAE, e.g. an attenuated androgen such as danazol, stanozolol, oxandrolone, methyltestosterone, tibolone, oxymetholone. In some embodiments, the treatment methods provided herein comprise adjunct administration of one or more of the following: a C1EI protein, optionally recombinant or plasma-derived, a kallikrein inhibitor, a bradykinin antagonist, and/or an attenuated androgen, for acute HAE attacks.

A “therapeutically effective amount” of a nucleic acid, vector construct, AAV particle, host cell, or a pharmaceutical composition comprising the same for purposes of treatment as described herein may be determined empirically and in a routine manner. In certain embodiments, however, a “therapeutically effective amount” of recombinant AAV particle ranges from about 1 × 10¹² to about 1 × 10¹⁴ or 1 × 10¹⁵ vg/kg. In another embodiment, the rAAV particle is delivered at about 2 x 10¹² to about 2 x 10¹⁴ vg/kg. In yet another embodiment, the rAAV particle is delivered at about 2 × 10¹² to about 6 × 10¹³ vg/kg. In yet another embodiment, the rAAV particle is delivered at about 1 × 10¹³ to about 1 × 10¹⁵ vg/kg.

In one embodiment, recombinant vector constructs or AAV particles provided herein may be administered to a subject, in one embodiment a mammalian subject, or a human subject, through a variety of known administration techniques. In some embodiments, the vector construct or recombinant AAV particle is administered by intravenous injection either as a single bolus or over a prolonged time period, which may be at least about 1, 5, 10, 15, 30, 45, 60, 75, 90, 120, 150, 180, 210 or 240 minutes, or more.

In any of the treatment methods described herein, the effectiveness of the treatment can be monitored by measuring levels of expressed functional C1EI levels in the blood of the treated subject. Precise quantitate assays for quantifying circulating levels of C1EI are well known in the art and include ELISA, Western blotting assays, fluorometric assays (see, McCaman, M.W. and Robins, E., (1962) J. Lab. Clin. Med., vol. 59, pp. 885-890); mass spectroscopy, thin layer chromatography based assays (see, Tsukerman, G. L. (1985) Laboratornoe delo, vol. 6, pp. 326-327); enzymatic assays (see, La Du, B. N., et al. (1963) Pediatrics, vol. 31, pp. 39-46; and Peterson, K., et al. (1988) Biochem. Med. Metab. Biol., vol. 39, pp. 98-104); methods employing high pressure liquid chromatography (HPLC) (see, Rudy, J. L., et al. (1987) Clin. Chem., vol. 33, pp. 1152-1154); and high-throughput automation (see, Hill, J. B., et al. (1985) Clin. Chem., vol. 5, pp. 541-546). Functional assays for confirming C1EI activity are commercially available, e.g. TECHNOCHROM® chromogenic kit in which C1-inh is titrated against an excess of C1-esterase to form an inhibitory complex, and the residual C1-esterase activity is measured using a chromogenic substrate. In addition, effectiveness of the treatment can be monitored with respect to reducing the frequency (number) of acute HAE attacks and/or severity of HAE attacks, and reduction in use of on-demand therapy for treating acute HAE attacks.

Administration of an AAV particle of the present disclosure may, in some cases, result in an observable degree of hepatotoxicity. Hepatotoxicity may be measured by a variety of well-known and routinely used techniques for example, measuring concentrations of certain liver-associated enzyme(s) (e.g., alanine transaminase, ALT) in the bloodstream of a subject both prior to AAV administration (i.e., baseline) and after AAV administration. An observable increase in ALT concentration after AAV administration (as compared to prior to administration) is indicative of drug-induced hepatotoxicity. In certain embodiments, in addition to administration of a therapeutically effective amount of AAV virus, the subject may be treated either prophylactically, therapeutically, or both with a corticosteroid to prevent and/or treat any hepatotoxicity associated with administration of the AAV virus.

“Prophylactic” corticosteroid treatment refers to the administration of a corticosteroid to prevent hepatotoxicity and/or to prevent an increase in measured ALT levels in the subject. “Therapeutic” corticosteroid treatment refers to the administration of a corticosteroid to reduce hepatotoxicity caused by administration of an AAV virus and/or to reduce an elevated ALT concentration in the bloodstream of the subject caused by administration of an AAV virus. In certain embodiments, prophylactic or therapeutic corticosteroid treatment may comprise administration of at least 5, 10, 15, 20, 25, 30, 35, 40, 45, 50, 55, 60, or more mg/day of the corticosteroid to the subject. In certain embodiments, prophylactic or therapeutic corticosteroid treatment of a subject may occur over a continuous period of at least about 3, 4, 5, 6, 7, 8, 9, 10 weeks, or more. Corticosteroids that find use in the methods described herein include any known or routinely-employed corticosteroid including, for example, dexamethasone, prednisone, fludrocortisone, hydrocortisone, and the like.

### Detection of Anti-AAV Antibodies

To maximize the likelihood of successful liver transduction with systemic AAV-mediated therapeutic gene transfer, prior to administration of an AAV particle in a therapeutic regimen to a human patient as described above, the prospective patient may be assessed for the presence of anti-AAV capsid antibodies or anti-AAV neutralizing antibodies that are capable of blocking cell transduction or otherwise reduce the overall efficiency of the therapeutic regimen. Such antibodies may be present in the serum of the prospective patient and may be directed against an AAV capsid of any serotype. In one embodiment, the serotype against which pre-existing antibodies are directed is AAV5.

Methods to detect pre-existing AAV immunity are well known and routinely employed in the art and include cell-based in vitro transduction inhibition (TI) assays, in vivo (e.g., in mice) TI assays, and ELISA-based detection of total anti-capsid antibodies (TAb) (see, e.g., Masat et al., Discov. Med., vol. 15, pp. 379-389 and Boutin et al., (2010) Hum. Gene Ther., vol. 21, pp. 704-712). TI assays may employ host cells into which an AAV-inducible reporter vector has been previously introduced. The reporter vector may comprise an inducible reporter gene such as GFP, etc. whose expression is induced upon transduction of the host cell by an AAV virus. Anti-AAV capsid antibodies present in human serum that are capable of preventing/reducing host cell transduction would thereby reduce overall expression of the reporter gene in the system. Therefore, such assays may be employed to detect the presence of anti-AAV capsid antibodies in human serum that are capable of preventing/reducing cell transduction by the therapeutic AAV-C1EI virus.

The assays to detect anti-AAV capsid antibodies may employ solid-phase-bound AAV capsid as a “capture agent” over which human serum is passed, thereby allowing anti-capsid antibodies present in the serum to bind to the solid-phase-bound capsid “capture agent”. Once washed to remove non-specific binding, a “detection agent” may be employed to detect the presence of anti-capsid antibodies bound to the capture agent. The detection agent may be an antibody, an AAV capsid, or the like, and may be detectably-labeled to aid in detection and quantitation of bound anti-capsid antibody. In one embodiment, the detection agent is labeled with ruthenium or a ruthenium-complex that may be detected using electrochemiluminescence techniques and equipment.

The same above-described methodology may be employed to assess and detect the generation of an anti-AAV capsid immune response in a patient previously treated with a therapeutic AAV virus of interest. As such, not only may these techniques be employed to assess the presence of anti-AAV capsid antibodies prior to treatment with a therapeutic AAV virus, they may also be employed to assess and measure the induction of an immune response against the administered therapeutic AAV virus after administration. As such, contemplated herein are methods that combine techniques for detecting anti-AAV capsid antibodies in human serum and administration of a therapeutic AAV virus for the treatment of HAE, wherein the techniques for detecting anti-AAV capsid antibodies in human serum may be performed either prior to or after administration of the therapeutic AAV virus.

Other aspects and advantages of the present disclosure will be understood upon consideration of the following illustrative examples.

### EXAMPLES

### Example 1: Evaluation of a C1ei Expression Cassette Driven by a Liver Specific Promoter

### Generation of Vectors Expressing Wild-Type Human C1EI Operably Linked to a Liver Specific Promoter

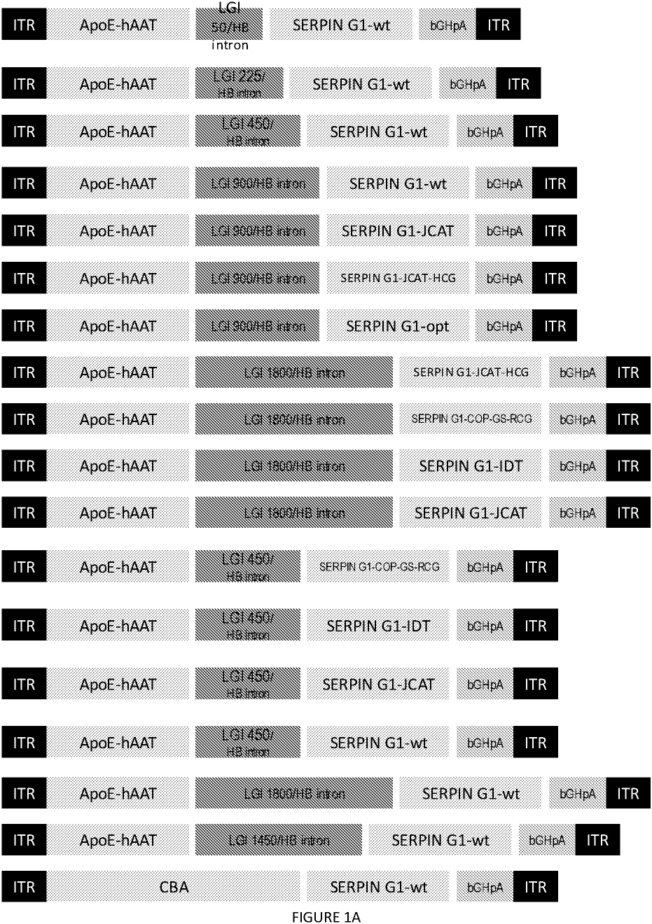

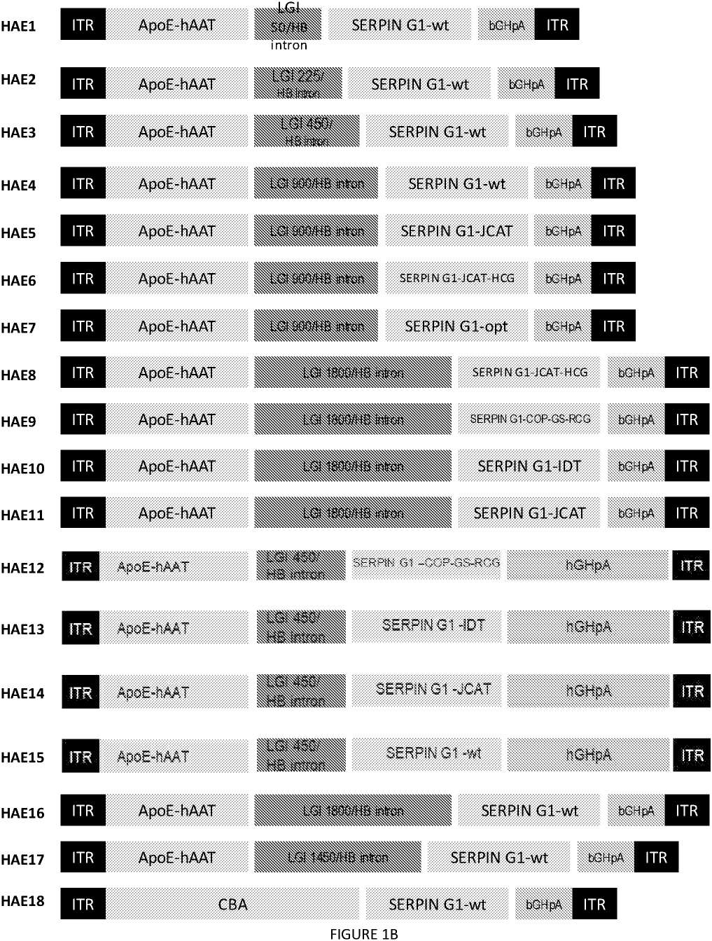

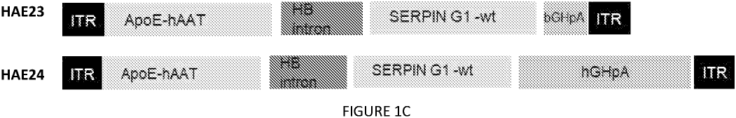

A variety of recombinant AAV gene therapy vectors were designed comprising a wild type or codon-optimized SERPING1 cDNA operably linked to a hybrid human apolipoprotein E (ApoE)/HCR enhancer / human alpha anti-trypsin (AAT) promoter (Table 1). Representative depictions of the vector configurations are further provided in FIG. 1. The vector genome configurations optionally included a hAAT/hemoglobin intron sequence (hhI), as well as a bovine or human growth hormone polyadenylation signal (bGHpA or hGHpA, respectively). The vector genomes are flanked by AAV serotype 2 (AAV2) derived inverted terminal repeats (ITRs) and ranged in size from 3087 bp to 4779 bp in length. The vectors were prepared using conventional cloning techniques as described e.g., by Gibson et al. (2009). “Enzymatic assembly of DNA molecules up to several hundred kilobases”. Nature Methods. 6 (5): 343-345, and Gibson DG. (2011). “Enzymatic assembly of overlapping DNA fragments”. Methods in Enzymology. 498: 349-361, which are incorporated herein by reference.

TABLE 1

AAV-C1EI Vector Constructs

| Code | Construct | SEQ ID NO: |
|---|---|---|
| HAE1 | pFB-ApoE-hAAT-LGI-50-SERPIN G1 | 20 |
| HAE2 | pFB-ApoE-hAAT-LGI-225-SERPIN G1 | 21 |
| HAE3 | pFB-ApoE-hAAT-LGI-450-SERPIN G1 | 22 |
| HAE4 | pFB-ApoE-hAAT-LGI-900- SERPIN G1 | 23 |
| HAE5 | pFB-ApoE-hAAT-LGI-900-SEPRIN G1-JCAT | 24 |
| HAE6 | pFB-ApoE-hAAT-LGI-900-SERPIN G1-JCAT-HCG | 25 |
| HAE7 | pFB-ApoE-hAAT-LGI-900-SERPIN G1-opt | 26 |
| HAE8 | pFB-ApoE-hAAT-LGI-SERPIN G1-JCAT-HCG | 27 |
| HAE9 | pFB-ApoE-hAAT-LGI-SERPIN G1-Cop-GS-RCG | 28 |
| HAE10 | pFB-ApoE-hAAT-LGI-SERPIN G1-IDT | 29 |
| HAE11 | pFB-ApoE-hAAT-LGI-SERPIN G1-JCAT | 30 |
| HAE12 | pFB-ApoE-hAAT-SERPIN G1-Cop-GS-RCG-hGHpA-4300 | 31 |
| HAE13 | pFB-ApoE-hAAT-SERPIN G1-IDT-hGHPA-4300 | 32 |
| HAE14 | pFB-ApoE-hAAT-SERPIN G1-JCAT-hGHPA-4300 | 33 |
| HAE15 | pFB-ApoE-hAAT-SERPIN G1-P1-hGHPA-4300 | 9 |
| HAE16 | pFB-ApoE-hAAT-LGI-SERPIN G1-WT | 34 |
| HAE17 | pFB-ApoE-hAAT-LGI-SERPIN G1-WT deletion marked for 4399 | 35 |
| HAE23 | pFB-ApoE-hAAT-HB intron-SERPIN G1-WT-bGHpA | 57 |
| HAE24 | pFB-ApoE-hAAT-HB intron-SERPIN G1-WT-hGHpA | 58 |

### Assays to Test the Expression and Activity of AAV-C1EI Vectors

Assays to test the recombinant AAV-C1EI vectors provided herein include, for example, (1) transient transfection of double-stranded DNA plasmids comprising the AAV vector nucleic acids in HepG2 cells, a cell line derived from human liver, to check liver-specific C1EI protein production and secretion in vitro; (2) production of AAV virions comprising the AAV-C1EI vectors in 293 cells and baculovirus-infected insect cells, followed by confirmation of the AAV-C1EI vector in 293 cells and baculovirus-infected insect cells, followed by confirmation of the AAV vector nucleic acids and capsid protein integrity; and (3) evaluation of C1EI expression and C1EI activity in Rag2⁻/⁻ mice.

### Transient Transfection Assays

A preliminary in vitro assay was performed to compare the C1EI expression and activity from the recombinant AAV gene therapy vectors described above (see also FIG. 1).

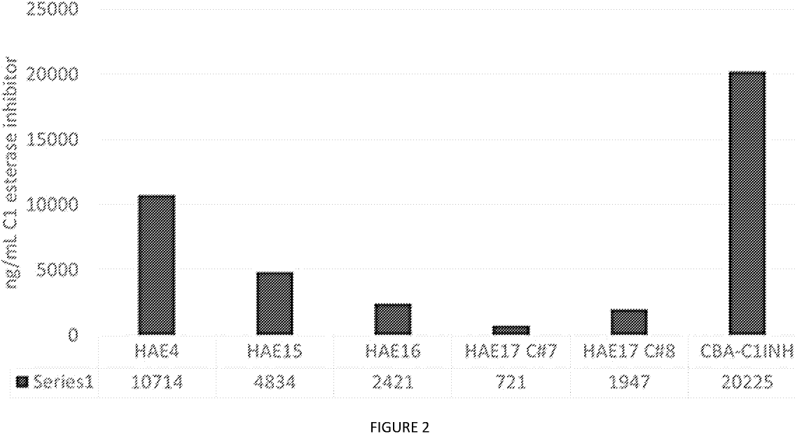

In one embodiment, plasmids of the vector constructs are transiently transfected into the human liver cell line, HepG2. After transfection, for example, 24 or 48 or 72 hours later, C1EI expression is measured. Using this assay, the recombinant AAV gene therapy vectors were demonstrated to be capable of expressing C1EI protein in transiently transfected HepG2 cells at levels of about 20220 to 721 ng/mL as depicted in FIG. 2.

### Production of AAV-C1EI Virions in 293 Cells and Baculovirus-Infected Insect Cells

To demonstrate that the recombinant vectors of the present embodiment indeed package the nucleic acids encoding C1EI, the double-stranded forms of the AAV-C1EI vectors generated as described above were introduced into cells capable of producing AAV virions. Baculovirus constructs were generated expressing the AAV-C1EI vector nucleic acids and the AAV Cap and Rep proteins, and then were co-infected into insect cells, preferably rhabdovirus-free, derived from Sf9 cells. The resultant AAV virions were purified and analyzed by standard methods known in the art. In an alternative AAV virus production system, plasmids comprising the AAV-C1EI vector nucleic acids in double-stranded form were co-transfected into 293 cells together with a plasmid that expresses the AAV Cap and Rep proteins and a plasmid that expresses adenovirus helper functions needed to for AAV virion production.

An alkaline gel electrophoresis assay was performed to determine the size of the packaged nucleic acid. The results showed that the nucleic acids were of the expected length. Alternate assays include a replication center assay to determine which AAV-C1EI vectors are packaged in an intact form. A primer extension assay is used to quantify the amount of AAV-C1EI vector nucleic acids that have complete ends, i.e., terminate at the 5′ end of the hairpin loop in the AAV2 5′ ITR (sense strand) or 3′ ITR (anti-sense strand). Alternatively, a PCR assay is used to determine whether the AAV-C1EI vectors nucleic acids have complete ends, i.e., terminate at the 5′ end of the hairpin loop in the AAV2 5′ ITR (sense strand) or 3′ ITR (anti-sense strand).

Transient transfection assays showed that the vector was capable of expressing high levels of exogenous C1EI in liver cells. AAV virions were produced generally as described herein using two different types of capsids, AAV5-type capsid and a baboon AAV-derived capsid. Evaluation of the thus-produced nucleic acids showed that they were of the expected length.

### Evaluation of Transducibility in HepG2 Cells

AAV virions as produced above using two different types of capsids, AAV5 type and a baboon AAV-derived (Bba49) capsid, were further evaluated via in vitro transduction ofHepG2 cells. HepG2 cells were transduced at 3 different MOIs 20,000; 100,000; and 500,000 with several preparations of AAV particles with AAV5 type or AAVBba49 capsids. 96 hours post transduction the media was collected and bCG protein was measured by mass spec. showing good transducibility and similar transduction results across all preparations.

### Example 2: Effect of Hae15 in Rag2⁻/⁻ MICE

AAV5 HAE15 (or AA V5-ApoE/HCR-hAAT.hhI.SERPIN G1.hGH) was produced using a baculovirus expression vector (BEV) system and another liver-tropic baboon-derived AAV capsid AAVBba49 HAE15 (or AAVBba-ApoE/HCR-hAAT.hhI.SERPIN G1.hGH) was produced using triple transfection of 293 cells. The purified vectors were quantified by qPCR and dosed at 2e14vg/kg into 8 week old Rag2-/- (knockout) mice alongside a vehicle control group. Post-dosing, serum and plasma was collected at weeks 2, 4, 6, 8, 10, and 12. Human C1EI levels were measured by a paired ELISA kit from Sino Biological Inc (Cat# SEK10995) and functional C1EI levels were measured using Technochrom C1-inh kit from DiaPharma (Cat# 5345003).

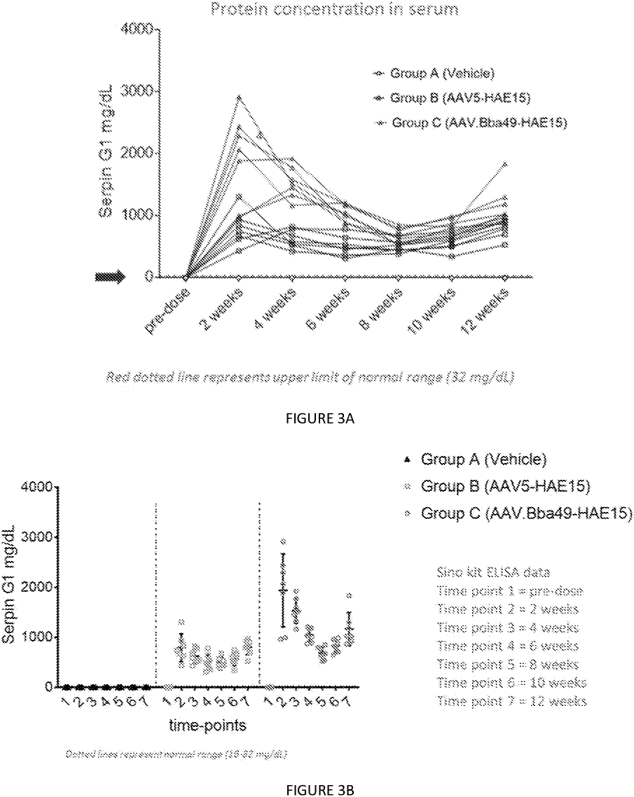

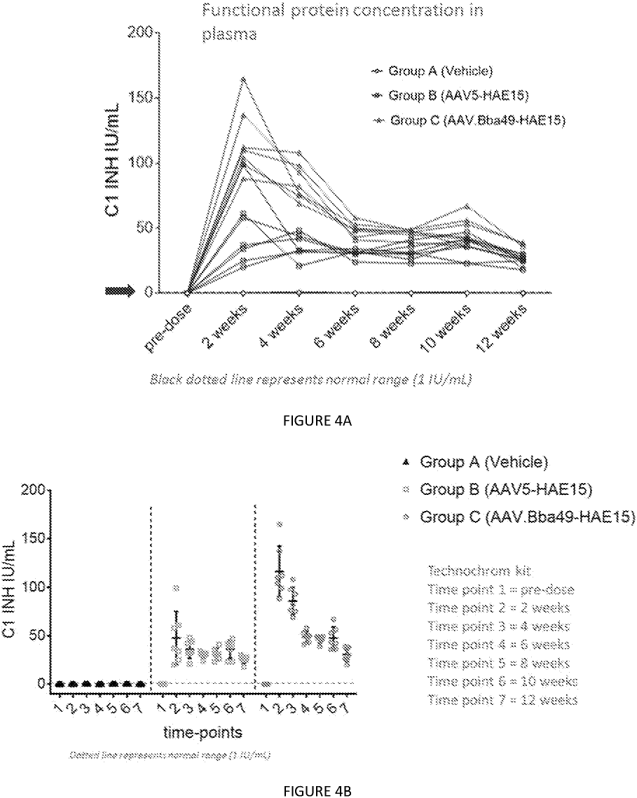

In both groups dosed with the two different rAAV particles there was supraphysiological levels of human C1EI expression (FIGS. 3A and B), as well as functional protein concentration in the serum (FIGS. 4A and B) well above vehicle background. The group dosed with AAVBba49-HAE15 had the highest protein expression with >50 times normal levels of functional human C1EI protein being secreted. In both AAV5 and AAVBba49 treated groups, a sustained expression was seen over the 12 week study. Expression of human C1EI levels in the AAVBba49 HAE15 group decreased from 2 to 6 weeks and stabilized by 6 weeks at higher levels 40-70IU/mL) of human C1EI protein expression compared to the AAV5 HAE15 treated group (25-48 IU/mL). Expression of C1EI protein detected in the plasma in Group B: 2e14vg/kg AAV5 HAE15 held steady from week 4 to week 12 (both protein quantification and functional C1EI levels). Group C: 2e14vg/kg AAVBba49 HAE15 plasma C1EI levels held steady from week 6 to week 12 (both protein quantification and functional C1EI levels). All remained high from 25-50x normal.

### Growth Rate

Growth rates were also measured in Rag2-/- mice treated with vehicle as controls and compared with the AAV-C1EI treated mice based on body weight measurements before plasma sample retrieval (FIG. 5). Weight graphs from all groups through 10 weeks demonstrate no significant change in the rate of gain in body weight.

### Measurement of Toxicity Indicators

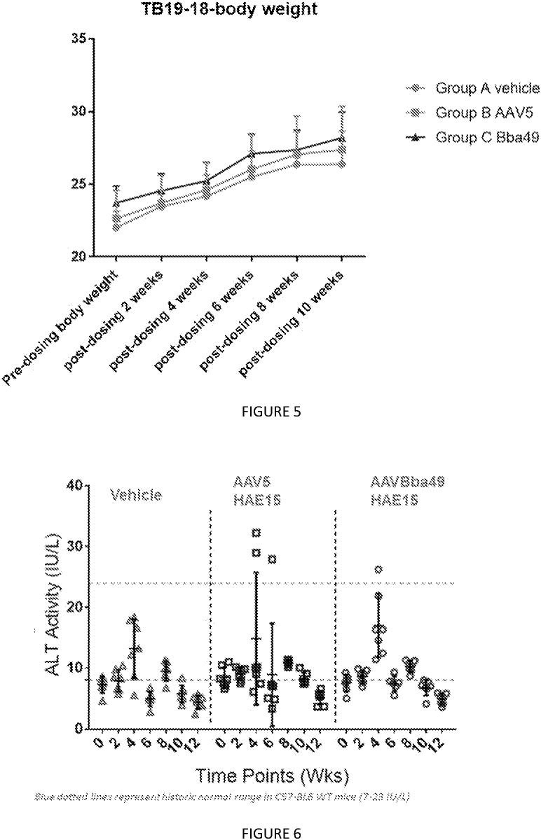

Alanine aminotransferase (ALT) activity in plasma can be used as an indicator of hepatocyte health, and higher levels of ALT are indicative of hepatocyte toxicity. Plasma samples were taken from these mice before administration of AAV5 or AAVBba49 treatment and every 2 weeks after administration (FIG. 6). Plasma ALT was measured using a commercial kit (Sigma). Graphs indicate that administration of either AAV5 or AAVBba49 with the HAE15 C1EI vector genome did not lead to appreciable change in ALT levels. The dashed lines represent historic normal range in C57-BL6 WT mice (7-23 IU/L).

### Histology of Rag2-/- Mice Treated with AAV5-HAE15 or Bba49-HAE15

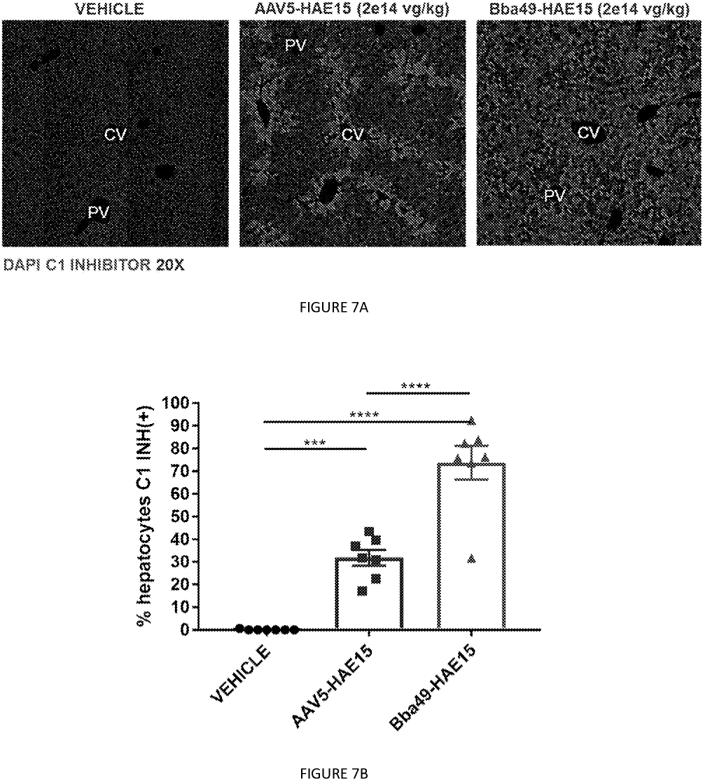

Livers of these treated mice were evaluated 12 weeks post-dose for histology and expression of C1EI. As depicted in FIG. 7A, dosing 8-week-old Rag2-/- mice with 2e14 vg/kg AAV5-HAE15 or Bba49-HAE15 results in increased hepatic expression of human C1EI. More specifically, peri-central signal was detected in AAV5-HAE, while a pan-liver signal was observed for Bba49-HAE15. Using a C1EI immunohistochemistry (IHC) assay, significant differences were observed between each construct, and compared to vehicle (FIG. 7B). % C1EI (+) hepatocytes were quantified based on a set threshold. Two to three regions of interest (2-3 ROIs) consisting of ~4600 +/- 770 hepatocytes were counted per animal. For the mice administered the 2e14 vg/kg dose of AAV5-HAE15, approximately 30% of the hepatocytes were C1EI positive, indicating that they were making vector derived human C1EI at 12 weeks post dose.

Dosing with AAV5 HAE15 or Bba49 HAE15 did not affect hepatic histo-architecture, based on qualitative assessment of factors such as enlargement of hepatic nuclei; collapse of hepatic sinusoids; and the presence of hepatic lesions. H&E tissues presenting with architectural pathology were utilized for comparative analysis.

IBA1 is a marker of both resident and infiltrating macrophages, and inclusive of basal and activated states. Dosing 8-week-old Rag2-/- mice with 2e14 vg/kg AAV5-HAE15 or Bba49-HAE15 results in elevated IBA-1(+) Foci within the livers of Rag2-/- mice. Using an IBA1 IHC assay, a significant increase was observed in IBA1 signal in both AAV5 and Bba49 dosed animals (increase approx. ~10%)(One-way ANOVA; Error Bar = SEM). The number of IBA-1 (+) foci were analyzed and divided by the total area of the image (excluding empty spaces) to calculate the #IBA-1 foci per pixel. Previous gene therapy projects have reported similar findings when dosing at 2e14vg/kg (and higher) concentrations.

### Liver DNA and RNA qPCR

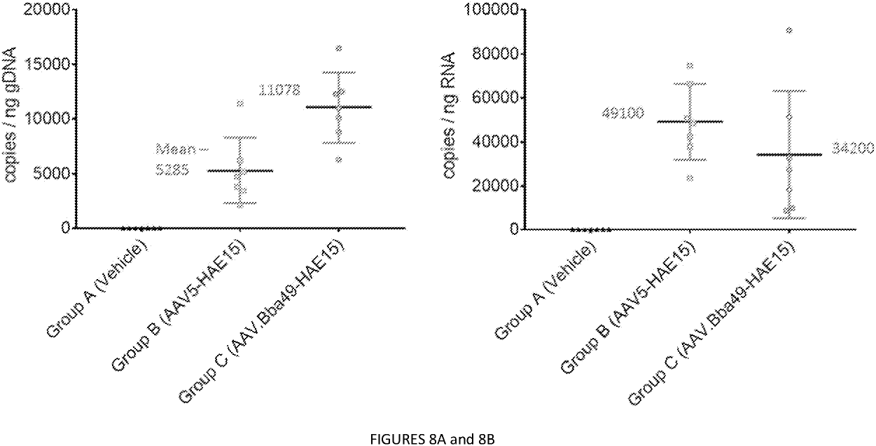

At 12 weeks post treatment the Rag2-/- mice dosed with 2e14 vg/kg of either AAV5-HAE15 or Bba49-HAE15 vector constructs were further evaluated for the HAE15 DNA and RNA in the liver as measured by qPCR. Fold difference between group B and group C RNA: group C/ group B - by copies / ng RNA - 0.69; by ΔΔct - 0.62 are depicted in FIG. 8. Dosing 8-week-old Rag2-/- mice with 2e14 vg/kg AAV5-HAE15 or Bba49-HAE15 results in increased HAE15 DNA and RNA in the liver of both treatment groups.

### Example 3: Dose Ranging Study

A dose response study was conducted with AAV5-HAE15 in two cohorts - the first cohort was evaluated for 12 weeks, and the second cohort was evaluated for 12 months.

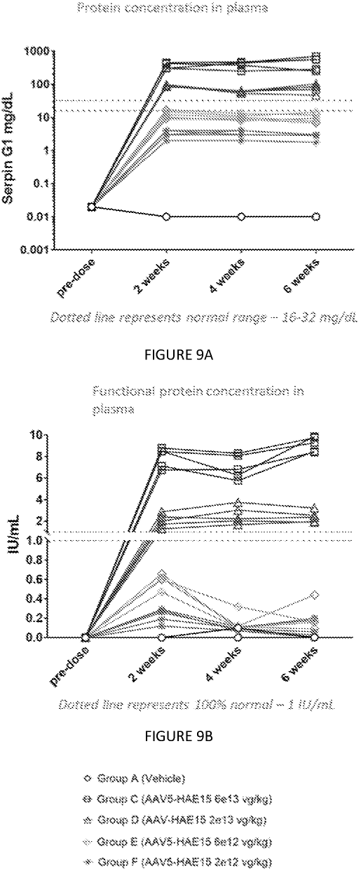

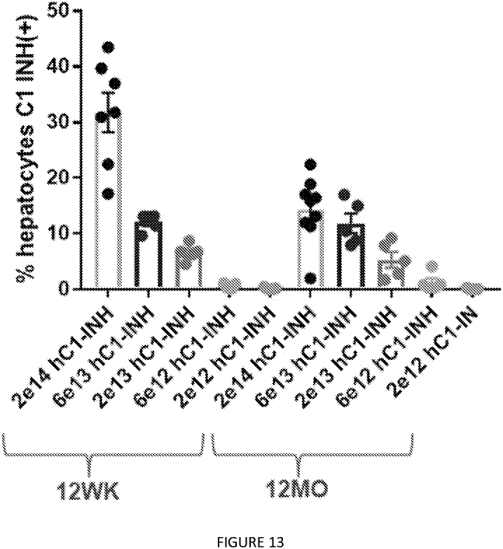

In the first cohort, 8 week old male Rag2-/- mice were treated with vehicle, or an AAV HAE15 dose of 6e13vg/kg; 2e13vg/kg; 6e12vg/kg; or 2e12vg/kg. Blood sampling and plasma collection was done by tail nick before injection and every two weeks post injection up to 12 weeks. At 12 weeks, the mice were evaluated for amounts of HAE15 DNA and RNA in the liver as measured by Droplet Digital PCR (ddPCR). Livers were also evaluated for histology and expression of C1EI by immunohistochemistry (IHC). At 2 weeks all treated groups had detectable levels of human C1EI levels with the 2 highest groups demonstrating supraphysiological levels of human C1EI protein expression (FIGS. 9A and B). A good dose response is seen with AAV5-HAE15. At 2 weeks post dose, the 6e13vg/kg dose group had average levels of 386 mg/dL and 7.94IU/mL human C1EI; the 2e13vg/kg dose group had average levels of 91 mg/dL and 2.06IU/mL human C1EI; the 6e12vg/kg dose group had average levels of 13 mg/dL and 0.59IU/mL human C1EI; the 2e12vg/kg dose had average levels of 3 mg/dL and 0.23IU/mL human C1EI. These supraphysiological levels were maintained in the 6e13vg/kg; 2e13vg/kg treatment groups at 4 and 6 weeks post treatment. Administration of AAV5-HAE15 also provided a dose-dependent increase in the amount of HAE15 DNA detected in liver (FIG. 12). Evaluation of C1EI(+) hepatocytes by IHC at 12 weeks post-administration showed a dose-dependent peri-central signal For mice administered the 2e13 vg/kg dose, approximately 7% of the hepatocytes were C1EI(+) at 12 weeks, while for mice administered the 6e13 vg/kg dose, approximately 12% of the hepatocytes were C1EI(+) at 12 weeks (FIG. 13).

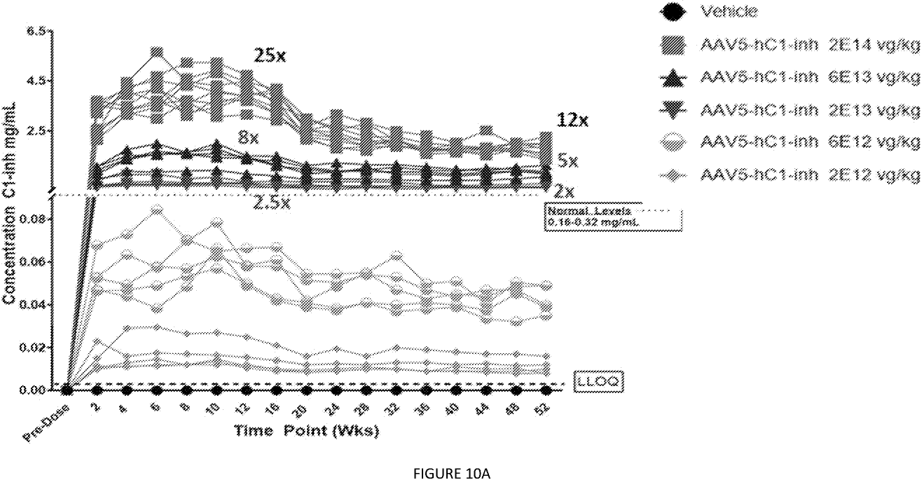

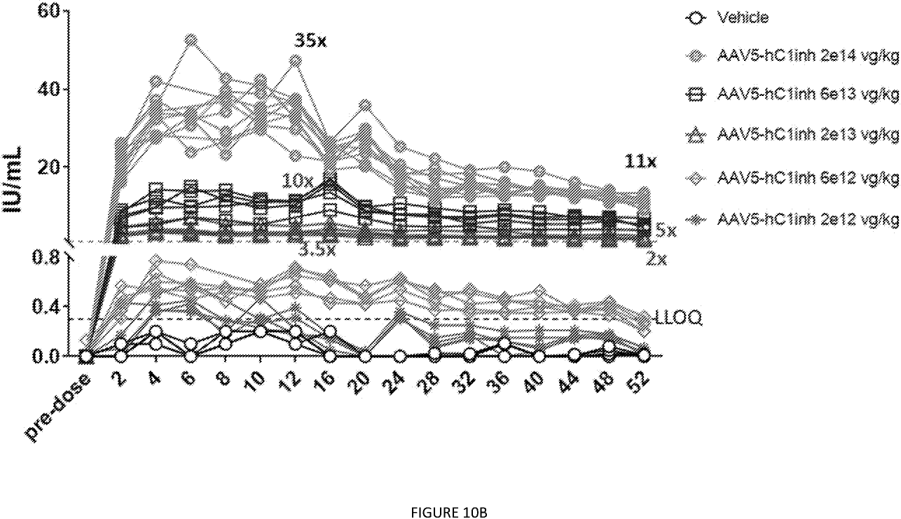

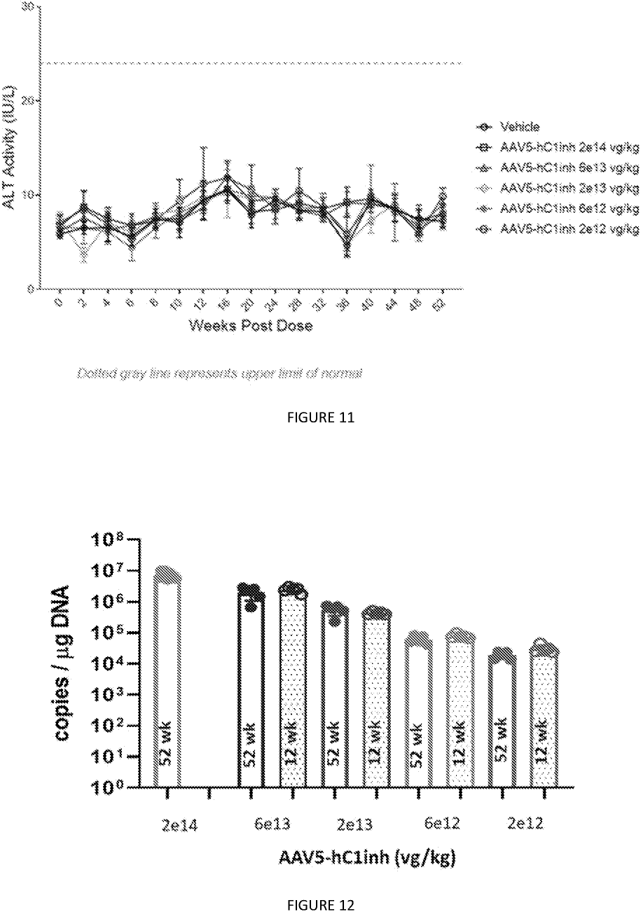

In the second cohort, 8 week old male Rag2-/- mice were treated with vehicle, or an AAV5-HAE15 dose of 2e14 vg/kg; 6e13vg/kg; 2e13vg/kg; 6e12vg/kg; or 2e12vg/kg. Blood and plasma were collected as described above every two weeks post injection up to 12 weeks, then every 4 weeks to week 52. Plasma was assessed for mg/mL of total human C1EI protein (FIG. 10A), IU/mL of functional C1EI protein (using an assay measuring C1 esterase inhibition) (FIG. 10B), and IU/L ALT, a biomarker indicative of liver toxicity (FIG. 11). The amount of human C1EI protein in mouse plasma was measured by liquid chromatography-mass spectrometry (LC-MS)/MS using a human-specific peptide TLYSS. Functional C1EI protein was measured with a commercially available kit (Technochrom). At 52 weeks, the mice were euthanized. Their livers were evaluated for amounts of HAE15 DNA and RNA (copies of HAE15 DNA per µg total DNA) as measured by Droplet Digital PCR (ddPCR) (FIG. 12). Livers were also evaluated for histology and expression of C1EI. The percentage of C1EI(+) hepatocytes were quantified based on a set threshold; vehicle treated animals were used to set the minimum threshold and to subtract out any background/ autofluorescent signal. Two to three ROI consisting of approximately 5000 +/- 580 hepatocytes were counted per animal.

Expression of both total human C1EI protein and functional C1EI protein was dose dependent and peaked between 4 and 12 weeks. The 2e13 vg/kg and higher doses all provided supraphysiological levels of human C1EI and functional human C1EI expression for at least a year. The human C1EI protein levels ranged from about 2.5x to 25x normal at 12 weeks, and gradually dropped to levels ranging from about 2x to about 12x normal at 52 weeks (FIG. 10A). The functional human C1EI levels followed a similar pattern, ranging from about 3.5x to about 35x normal at 12 weeks, and dropping to levels of about 2x to about 11x at 52 weeks (FIG. 10B). The levels of total C1EI protein and functional C1EI protein were well correlated.

These data indicate that the AAV5-HAE15 vector induced therapeutic levels of functional C1EI expression, with good durability over the one-year evaluation period, with sufficient levels expected to be expressed beyond the one year.

Administration of AAV5-HAE15 provided a dose-dependent increase in the amount of HAE15 DNA detected in liver at 52 weeks (FIG. 12). Evaluation of C1EI(+) hepatocytes by IHC at 52 weeks post-administration showed the expected dose-dependent signal, at levels comparable to the 12 week study of the first cohort (FIG. 13). At 52 weeks, approximately 5% of the hepatocytes remained C1EI positive with the 2e13 vg/kg dose, approximately 12% of the hepatocytes remained C1EI positive with the 6e13 vg/kg dose, and approximately 15% of the hepatocytes remained C1EI positive with the 2e14 vg/kg dose. For the 2e13 vg/kg and 6e13 vg/kg doses, there was no significant difference in percentage of C1EI(+) hepatocytes at 12 weeks vs. 52 weeks.

This data indicates good, consistent durability of hepatocyte transduction through the 52-week duration of the study, with durability of expression expected to continue beyond the one year.

ALT levels remained within normal range for all doses through the 52-week duration of the study, indicating normal liver function. The treated mice showed no significant change in the rate of gain in body weight, and no significant liver histology findings. This data indicates that the AAV vector administration was safe and tolerable.

### Example 4: Evaluation of AAV-c1ei Vectors in Non-Human Primates

A non-human primate study was conducted with 16 cynomolgus monkeys (Macaca fascicularis). The monkeys were administered vehicle or (a) a low dose of AAV5-SERPING1 vector encoding either cynomolgous C1EI (cC1EI) or human C1EI (hC1EI) each at approximately 2e14 vg/kg, or (b) or a higher dose of AAV5-SERPING1 vector encoding either cC1EI (approximately 6.5e14 vg/kg) or hC1EI (approximately 5e14 vg/kg). AAV5-HAE15 was administered as the vector encoding human C1EI. Plasma was collected weekly for 13 weeks (low dose) to 17 weeks (high dose) and assessed for hC1EI protein levels. At the end of the study, the amount of SERPING1 DNA and RNA in the liver was assessed (copies of DNA or RNA/µg total DNA or RNA, respectively). Clinical pathology and hematology readouts are monitored.

Both the high and low doses of AAV5-HAE15 produced efficacious levels of human protein, although lower levels than seen in mice. There was a trend towards a dose-dependent response, while fluctuating within the normal range of C1EI levels. Vector DNA copies corresponded to protein expression levels, and vector RNA copies trended to correspond to protein expression levels. For example, treatment with 3x more AAV particles produced 3x more copies of DNA in the livers.

Safety endpoints include weekly physical, and body weight measurements, as well as monitoring for anti-AAV5 antibody and anti-C1EI antibody responses. The primates are monitored for swelling and if seen additional analyses are performed. Thrombotic endpoints include APTT, PT, soluble fibrin, D-dimer, thrombin-anti-thrombin complex, fibrinogen. At the time of study termination gross necropsy is performed and the liver assessed for C1EI, while all major organs (including gonads) are assessed for H&E and fibrin stains.

Table 2 - Blood collection for biomarkers (baseline, weekly, study termination)
❖ Liver enzymes
   aspartate aminotransferase (AST)
   alanine aminotransferase (ALT)
❖ Evaluation of thrombosis:
   APTT (clotting performance by intrinsic pathway)
   PT (clotting performance by extrinsic pathway)
   D-Dimer (marker of presence of fibrin clots)
   Soluble fibrin monomer (marker of disseminated intravascular clotting)
   Thrombin-antithrombin complex (marker of hypercoagulative state)
   Fibrinogen (marker of inflammation and perturbations could indicate fibrin-clot formation)

No abnormal results were observed for liver enzymes, coagulation parameters, or inflammation markers showing that non-human primates tolerated high doses of AAV5 vector with no adverse effects.

### Example 5: Evaluation of AAV-c1ei Vectors in Mouse Model of Hereditary Angioedema

AAV-SERPING1 directed expression of hC1EI protein is assessed in a mouse model of HAE. SERPING1^{-/-} mice have markedly increased vascular permeability compared to wild-type mice that mimics the symptoms of HAE. Following injection with Evans blue dye, homozygous CIEI-deficient mice exhibit increased vascular permeability in comparison with wild-type littermates. This increased vascular permeability can be reversed by treatment with intravenous C1EI, with a kallikrein inhibitor, or with a bradykinin type 2 receptor antagonist. Han et al., J. Clin. Invest. 109:1057-1063 (2002).

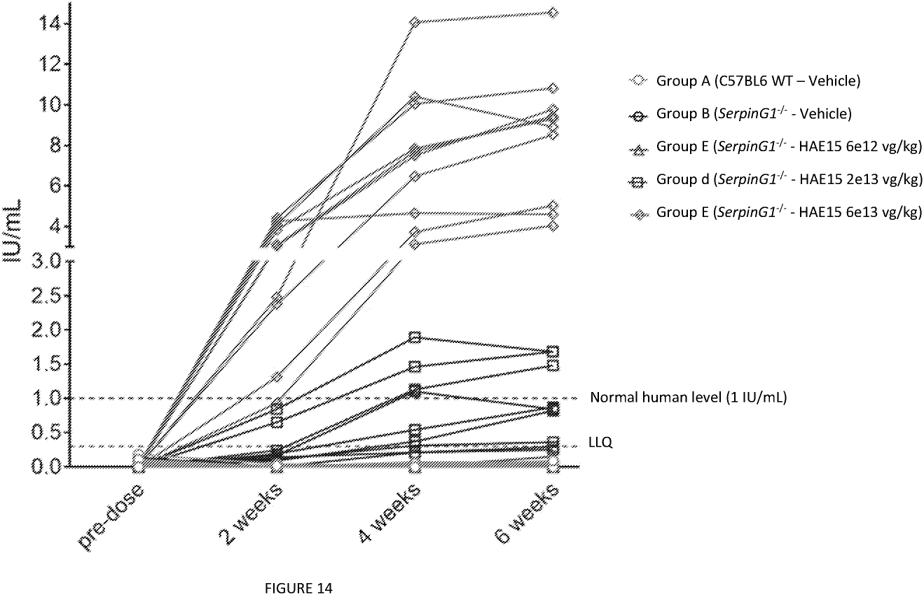

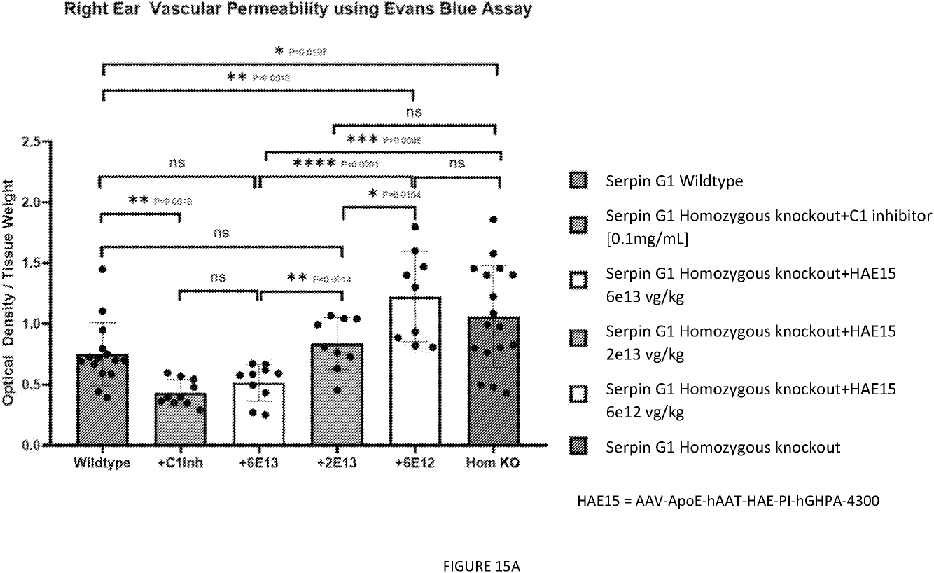

The therapeutic effect of AAV5-HAE15 was evaluated in homozygous SERPING1^{-/-} mice, age 6 to 8 weeks. Mice were treated with vehicle, with a positive control (4 mg/kg human C1EI protein), or with an AAV5-HAE15 dose of 6e13vg/kg; 2e13vg/kg; or 6e12vg/kg. Injections were performed intravenously at 4 µl/g body weight. Blood from the tail vein was collected at 2 week intervals over a 6-week period, processed to collect serum and assessed for levels and activity of hC1EI. Vector treated mice were compared to vehicle treated mice as controls. Functional human C1EI levels in plasma reached approximately therapeutic levels by week 4 to week 6 for both the 6e13vg/kg and 2e13vg/kg dose groups (FIG. 14). For the 6e13 vg/kg dose group, expression of functional human C1EI was substantially higher than normal for all mice in the group, ranging from about 4x to about 14x normal. Evaluation of C1EI(+) hepatocytes in the liver by IHC showed approximately 10% of the hepatocytes were C1EI(+) at 6 weeks with the 6e13 vg/kg dose.

At 2 weeks, 4 weeks or 6 weeks post-administration of AAV5-HAE15, groups of mice were also evaluated for vascular permeability. Mice were injected with 30 mg/kg Evans blue dye in phosphate buffered saline (PBS), followed by two applications over 15 minutes of an irritant (5% mustard oil) to the right ear surface. At 30 minutes after injection of the dye, the mice were euthanized. The dye was extracted from the right ear, small intestine and kidney, and measured spectrophotometrically at 600 nm.

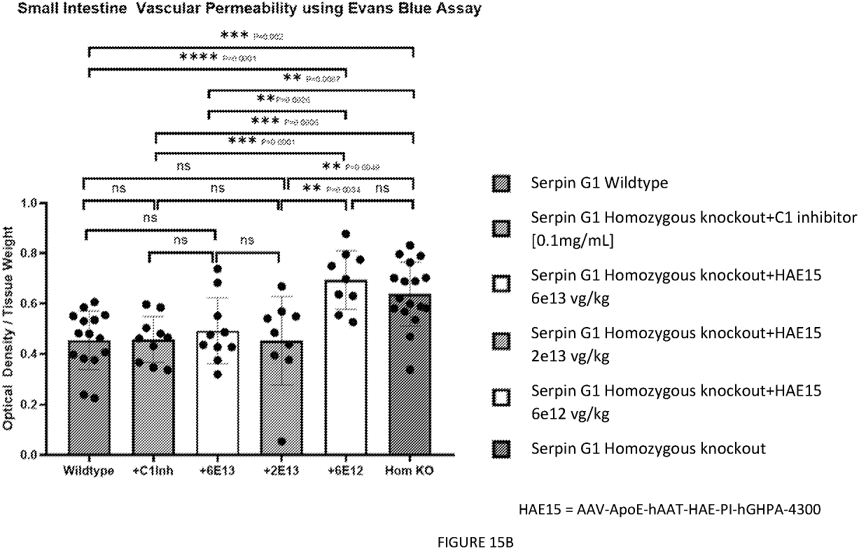

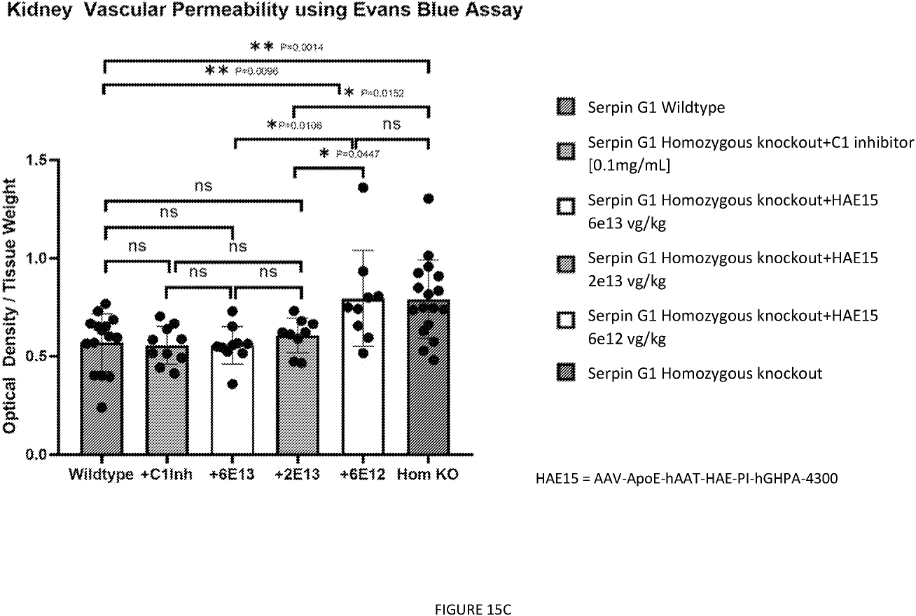

Control SERPING1 knockout mice treated with vehicle had significantly increased vascular permeability compared to the wild-type mice. As expected, treatment of mice with human plasma-derived C1EI protein normalized plasma levels of functional C1EI (approximately 1 IU/mL) and significantly reduced vascular permeability in the ear, small intestine, and kidney. In the ear pinna, the mice showed a dose-dependent response to administration of AAV5-HAE15. The vascular permeability in the ear pinna of mice dosed with 2e13 vg/kg was normalized (not significantly different to wild-type), while mice dosed with 6e13vg/kg showed even less vascular permeability compared to wild-type mice. (FIG. 15A). In the small intestine and kidney (FIGS. 15B and 15C), the vascular permeability of mice dosed with either 2e13 vg/kg or 6e13 vg/kg was normalized (not significantly different to wild-type).

### Example 6: Evaluation of Toxicity and Biodistribution of HAE15 in Cynomolgus Monkeys

A non-human primate study is conducted with cynomolgus monkeys, on HAE15, HAE23 or HAE24. Acute and chronic toxicity, pharmacodynamic and immunogenicity endpoints are followed throughout study. At 8 and 12-weeks post injection durability and chronic effects are assessed. Biodistribution is calculated, histopathology is assessed, and DNA and RNA in gonads are evaluated by in situ hybridization. The AAV vector is determined to be safe and tolerable.

The embodiments described herein are intended to be merely exemplary, and those skilled in the art will recognize, or will be able to ascertain using no more than routine experimentation, numerous equivalents of specific compounds, materials, and procedures. All such equivalents are considered to be within the scope of the disclosure.

All of the patents, patent applications and publications referred to herein are incorporated by reference herein in their entireties. Citation or identification of any reference in this application is not an admission that such reference is available as prior art to this application. The full scope of the disclosure is better understood with reference to the appended claims.

1. A recombinant vector construct comprising a nucleic acid sequence that encodes a functional C1 esterase inhibitor (C1EI) operably linked to a heterologous liver-specific transcription regulatory region, optionally a nucleic acid sequence comprising SEQ ID NO: 1, 10, 11, 12, 13, 59 or 60.

2. The vector construct of claim 1, wherein the mammal is a human and the C1EI is human C1EI.

3. The vector construct of claim 1 or 2 wherein the functional C1EI comprises an amino acid sequence at least 95% identical to amino acids 23-500 of SEQ ID NO: 2.

4. The vector construct of any of the preceding claims, wherein the liver-specific transcription regulatory region comprises a synthetic promoter sequence comprising portions of an hAAT promoter and an HCR enhancer/ApoE enhancer.

5. The vector construct of claim 4, wherein the liver-specific transcription regulatory region comprises (a) a shortened ApoE enhancer sequence at least 90% identical to SEQ ID NO: 16; (b) an alpha anti-trypsin (hAAT) proximal promoter sequence at least 90% identical to SEQ ID NO: 3, and/or (c) one or more enhancers selected from the group consisting of (i) an ApoE/HCR enhancer at least 90% identical to SEQ ID NO: 4.

6. The vector construct of claim 4, wherein the liver-specific transcription regulatory region comprises (a) an a-microglobulin enhancer sequence at least 90% identical to SEQ ID NO: 17, and (b) an alpha anti-trypsin (AAT) proximal promoter at least 90% identical to SEQ ID NO: 3.

7. The vector construct of claim 4, wherein the liver-specific transcription regulatory region comprises a nucleotide sequence at least 80% identical to SEQ ID NO: 5.

8. The vector construct of any of the preceding claims further comprising a polyadenylation signal.

9. The vector construct of claim 8 wherein the polyadenylation signal is a human growth hormone polyadenylation signal or functional fragment thereof.

10. The vector construct of any of the preceding claims further comprising an intron.

11. The vector construct of claim 10 wherein the intron is a composite hAAT/hemoglobin intron sequence.

12. The vector construct of claim 10, wherein the intron comprises a nucleotide sequence at least 80% identical to any of SEQ ID NOs: 6 or 61-69.

13. The vector construct of any of claims 10-12 wherein the nucleic acid sequence that encodes the functional C1 esterase inhibitor (C1EI) comprises the intron.

14. The vector construct of any of the preceding claims further comprising an AAV 5′ ITR and/or AAV3′ ITR from AAV2.

15. The vector construct of claim 1 that comprises a nucleotide sequence at least 80% identical to any one of SEQ ID NOs: 9, 20-36, 57 or 58.

16. The vector construct of any of the preceding claims that is an rAAV vector construct about 2.7 kb to about 4 kb, or about 4 kb to about 5 kb in size.

17. An rAAV particle comprising the vector construct of any of the preceding claims and an AAV capsid.

18. The rAAV particle of claim 17 that comprises an AAV5 type capsid.

19. The rAAV particle of claim 17 that comprises a simian AAV capsid.

20. The rAAV particle of claim 17 that comprises a baboon-derived AAV capsid.

21. The rAAV of any of claims 17-20 wherein the rAAV particle comprises an AAV capsid with liver tropism.

22. A method of producing an rAAV particle comprising the steps of (a) providing a cell permissive for AAV replication with one or more nucleic acid constructs comprising: (i) a recombinant vector construct comprising (1) at least one AAV ITR, (2) a heterologous liver-specific transcription regulatory region, and (3) a nucleic acid encoding a functional C1EI, (ii) a nucleotide sequence encoding one or more AAV Rep proteins which is operably linked to a promoter that is capable of driving expression of the Rep protein(s) in the cell; and (iii) a nucleotide sequence encoding one or more AAV capsid proteins which is operably linked to a promoter that is capable of driving expression of the capsid protein(s) in the cell; (b) culturing the cell under conditions permitting expression of the Rep and the capsid proteins; and optionally (c) recovering the AAV particle.

23. The method of claim 22, wherein the cell is an insect cell.

24. The method of claim 22, wherein the cell is a mammalian cell.

25. The method of claim 22 wherein the cell is provided with a recombinant vector construct of any of claims 1-16.

26. A population of rAAV particles produced by the method of any one of claims 22-25, optionally enriched for particles comprising full length or nearly full-length vector genomes by steps that reduce the number of empty capsids.

27. A pharmaceutical composition comprising the vector construct of any of claims 1-16 or the rAAV particle of any of claims 17-21 or the population of rAAV particles of claim 26 in an aqueous suspension with a sterile pharmaceutically acceptable excipient.

28. A method of treating hereditary angioedema in a mammal, or treating or preventing any symptom thereof, comprising administering a therapeutically effective amount of the vector construct of any of claims 1-16 or the rAAV particle of any of claims 17-21 or the population of rAAV particles of claim 26 or the pharmaceutical composition of claim 27.

29. A method of treating hereditary angioedema in a mammal, or treating or preventing any symptom thereof, comprising administering a therapeutically effective amount of an rAAV particle comprising a vector construct that comprises a nucleic acid sequence that encodes a functional C1EI, optionally linked to a heterologous transcription regulatory element.

30. The method of claim 29 wherein the C1EI is a functional human C1EI that comprises an amino acid sequence at least 95% identical to amino acids 23-500 of SEQ ID NO: 2.

31. The method of any of claims 28-30, wherein the method reduces the frequency or severity of submucosal or subcutaneous edema in the mammal.

32. A method of expressing C1EI in the liver of a mammal, comprising administering an amount of the vector construct of any of claims 1-16 or the rAAV particle of any of claims 17-21 or the population of rAAV particles of claim 26 or the pharmaceutical composition of claim 27 effective to increase the level of C1EI expression in the liver of the mammal.

33. A method of increasing the level of functional C1EI in the blood of a mammal, comprising administering an amount of the vector construct of any of claims 1-16 or the rAAV particle of any of claims 17-21 or the population of rAAV particles of claim 26 or the pharmaceutical composition of claim 27 effective to increase the level of functional C1EI in the blood of a mammal.

34. A method of treating a deficiency in functional C1EI in a mammal, comprising administering an amount of the vector construct of any of claims 1-16 or the rAAV particle of any of claims 17-21 or the population of rAAV particles of claim 26 or the pharmaceutical composition of claim 27 effective to increase the level of functional C1EI in the blood of a mammal.

35. The method of claim 33 or 34 wherein the amount is effective to increase the level of functional C1EI to at least about 0.4 IU/ml, or 1 IU/ml or higher, or about 16 mg/dL or higher.

36. The method of any of claims 28-35, wherein the rAAV particle or vector construct is administered intravenously.

37. The method of any of claims 28-36, wherein the mammal is concurrently administered corticosteroid therapy with the vector construct or rAAV particle or pharmaceutical composition.

38. The method of any of claims 28-37 wherein the mammal is administered the rAAV particle at a dose ranging from about 1 x 10¹² to about 1 x 10¹⁵ vg/kg.

39. The vector construct or rAAV particle or pharmaceutical composition of any of the preceding claims for use in treatment of HAE or for use in treating a deficiency in functional C1EI or for use in increasing blood levels of functional C1EI in a mammal.


SEQUENCE LISTING (72 sequences)

SEQ ID NO: 1 - DNA, 1503 bp, Homo sapiens
atggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctga

SEQ ID NO: 2 - protein, 500 aa, Homo sapiens
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 3 - DNA, 405 bp, Artificial Sequence
  Synethic polynucleotide
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagta

SEQ ID NO: 4 - DNA, 321 bp, Artificial Sequence
  Synethic polynucleotide
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 5 - DNA, 725 bp, Artificial Sequence
  Synethic polynucleotide
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 6 - DNA, 611 bp, Artificial Sequence
  Synethic polynucleotide
gtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaatt

SEQ ID NO: 7 - DNA, 1129 bp, Artificial Sequence
  Synethic polynucleotide
ctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcctgggcctagggctgtgccagctgcctcgtcccgtcaccttctggcttcttctctccctccatatcttagctgttttcctcatgagaatgttccaaattcgaaatttctatttaaccattatatatttacttgtttgctattatctctgcccccagtagattgttagctccagaagagaaaggatcatgtcttttgcttatctagatatgcccatctgcctggtacaatctctggcacatgttacaggcaacaactacttgtggaattggtgaatgcatgaatagaagaatgagtgaatgaatgaatagacaaaaggcagaaatccagcctcaaagagcttacagtctggtaagaggaataaaatgtctgcaaatagccacaggacaggtcaaaggaaggaggggctatttccagctgagggcaccccatcaggaaagcaccccagacttcctacaactactagacacatctcgatgcttttcacttctctatcaatgg

SEQ ID NO: 8 - DNA, 3765 bp, Artificial Sequence
  Synethic polynucleotide
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaacctgcaggccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 9 - DNA, 4280 bp, Artificial Sequence
  Synethic polynucleotide
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgacctcgagcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcctgggcctagggctgtgccagctgcctcgtcccgtcaccttctggcttcttctctccctccatatcttagctgttttcctcatgagaatgttccaaattcgaaatttctatttaaccattatatatttacttgtttgctattatctctgcccccagtagattgttagctccagaagagaaaggatcatgtcttttgcttatctagatatgcccatctgcctggtacaatctctggcacatgttacaggcaacaactacttgtggaattggtgaatgcatgaatagaagaatgagtgaatgaatgaatagacaaaaggcagaaatccagcctcaaagagcttacagtctggtaagaggaataaaatgtctgcaaatagccacaggacaggtcaaaggaaggaggggctatttccagctgagggcaccccatcaggaaagcaccccagacttcctacaactactagacacatctcgatgcttttcacttctctatcaatgggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 10 - DNA, 1503 bp, Artificial Sequence
  Synethic polynucleotide
atggcctccagactgaccctcctcacactgctcctgctgctccttgctggagatagagccagctccaaccccaatgccacaagcagcagcagccaggaccctgagagcctgcaggacagaggagagggcaaggtggccacaacagtgatctctaaaatgctcttcgttgaacccatcctggaagtgtcctctctgcctaccacaaacagcaccacaaattcagccaccaagatcacagccaataccacagatgagcccacaacccaacctaccacagaaccaacaactcagcctacaatccagcctactcagccaaccacccagctgcctacagacagccccacacagcctacaacaggcagcttctgccctggacctgtgaccctgtgcagtgatctggaatctcactctacagaggctgtgctgggagatgccctggtggacttcagcctgaaactgtatcacgccttctctgccatgaaaaaggtggagacaaatatggcttttagccctttttctattgccagcctgctgacccaagtgctgctgggagctggagagaacacaaagaccaacttggaaagcatcctgagctaccctaaggacttcacctgtgtgcaccaggccctgaagggcttcacaaccaagggagtgacaagtgtttcccaaatcttccacagccctgatctggccatcagagacacctttgtgaatgcctccagaaccctgtacagcagctctcctagagtgctgagcaacaactctgatgccaacctggaactgatcaacacctgggtggccaaaaacacaaacaacaagatcagcagactgcttgatagcctcccttctgatactagactggtcctgctgaatgctatctacctgtctgctaagtggaagaccacttttgaccccaagaaaaccagaatggaacctttccactttaagaacagtgtgatcaaggtgcctatgatgaactccaagaaataccctgtggcccacttcattgaccagaccctgaaggccaaggtgggccagctgcaactgagccacaacctgagcctggtgattctggttcctcagaacctgaagcacagactggaagatatggagcaggccctgtctccatctgtgttcaaggccatcatggagaagctggaaatgagcaagttccagcccaccctgctgacactccccagaatcaaggtgaccaccagccaggatatgctgtctatcatggaaaagcttgagttctttgacttctcctatgacctgaacctctgtggcctgacagaagatccagacctccaggtgtctgccatgcagcaccagacagtgttggagctgacagaaacaggagtggaagctgcagctgcctctgccatctctgtggccagaacactgctggtgtttgaggtgcagcagcctttcctgtttgtgctgtgggaccaacagcacaagttccctgtgtttatgggcagagtgtatgaccctagagcctga

SEQ ID NO: 11 - DNA, 1503 bp, Artificial Sequence
  Synethic polynucleotide
atggccagcagacttaccctgcttaccctgctcttgcttctgttggctggagacagagccagctccaatcctaatgctacctccagctccagtcaagatccagagagcctgcaggatagaggagagggcaaggtggccaccacagtcataagtaaaatgctctttgtggaacccatcctggaggtgagttctcttcctaccactaactctaccacaaactctgcaaccaagatcacagccaatactacagatgagcctacaactcaaccaactacagaacccacaactcaacctacaatccagcctactcaacccactacacaactgcccactgactcacctacccagcctaccacagggtcattttgtccagggccagttaccctgtgtagtgaccttgagagccatagtacagaggctgttctgggggatgctcttgtagattttagccttaaactgtaccatgctttcagtgctatgaaaaaagttgaaacaaacatggctttttccccttttagtattgcttctctccttactcaggttcttctgggagctggggagaacacaaaaactaatcttgaatctatcctcagctatcctaaagactttacttgtgtgcaccaagctcttaaaggatttacaactaagggagttactagtgtgagtcagatttttcacagtccagaccttgctatcagagatacatttgttaatgcttcaagaactctgtatagtagcagtcccagggtcttgtcaaataacagtgatgccaatcttgagctcataaatacctgggtggctaagaatacaaacaataaaatatcaaggcttctggactctctcccaagtgataccagacttgttctcctgaatgcaatatacctcagtgctaaatggaaaactacatttgatcccaaaaaaactagaatggagccatttcactttaaaaatagtgtaatcaaggtacctatgatgaacagcaaaaaatatcctgtagctcactttattgaccaaactctcaaagccaaagtggggcaactccagctgagtcacaacctgagtttggtaattctggtaccacaaaatctgaagcacagacttgaagatatggaacaagctctctctcccagtgtttttaaagccataatggaaaagcttgaaatgagtaaatttcaacccacactcctcaccctccctaggatcaaagttaccacatcccaggacatgctcagtataatggagaaacttgagttctttgattttagctatgatctgaatctttgtggcctgacagaggatccagatctccaggtatctgccatgcagcaccagactgtgctggagctgacagaaacaggtgtagaagctgcagcagcctcagctataagtgtggctagaacattgcttgtctttgaggtccaacagccttttttgtttgttctttgggaccaacagcataaatttcctgtctttatgggaagagtttatgacccaagagcttaa

SEQ ID NO: 12 - DNA, 1503 bp, Artificial Sequence
  Synethic polynucleotide
atggccagcagacttaccctgcttaccctgctcttgcttctgttggctggagacagagccagctccaatcctaatgctacctccagctccagtcaagatcccgagagcctgcaggatagaggagagggcaaggtggccaccacagtgatcagcaagatgctgtttgtggagcctatcctggaggtgagctctctgcctaccacaaatagcaccacaaattctgccaccaagatcacagccaataccactgatgaacccactacccagcccacaactgagcctacaacccaacctactatccaacctacccagcctaccactcaactgcccactgacagccctacccaacctacaactggctccttctgccctggccctgtgaccctgtgcagtgatctggagagccactctactgaggctgtgctgggagatgctctggtggacttcagcctgaagctgtaccacgccttctctgctatgaagaaggtggagaccaatatggctttcagccccttctctattgccagcctgctgacccaagtgctcctgggagctggagagaataccaagaccaatctggagtctatcctgagctaccccaaggatttcacctgtgtgcaccaagccctgaagggcttcaccacaaagggagtgacctctgtgagccagatcttccacagcccagatctggctatcagagatacctttgtgaatgccagcagaaccctgtacagctcttcccctagagtgctgagcaacaattctgatgctaatctggagctgatcaatacctgggtggccaagaataccaacaataagatcagcagactcctggactccctgccctctgataccagactggtgctcctgaatgctatctacctgtctgccaagtggaagaccacttttgaccccaagaagaccagaatggagcccttccacttcaagaattctgtgatcaaggtgcccatgatgaatagcaagaagtaccctgtggcccacttcattgatcagaccctgaaggctaaggtgggccaactccagctgagccacaatctcagcctggtgatcctggtgccccagaatctgaagcacagactggaggatatggagcaagccctgagcccctctgtgttcaaggctattatggaaaagcttgaaatgagcaagttccagcccacactcctgaccctgcccagaatcaaggtgaccactagccaagatatgctgtctattatggaaaaactggaattctttgacttcagctatgacctgaatctgtgtggcctgactgaagatcccgacctgcaagtgtctgccatgcagcaccaaactgtgctggagctcactgagacaggagtggaggctgcagctgcttctgccatctctgtggccagaaccctcctggtgtttgaggtgcaacagcctttcctgtttgtgctgtgggatcagcaacacaagttccctgtgttcatgggcagagtgtatgatcccagagcctaa

SEQ ID NO: 13 - DNA, 1503 bp, Artificial Sequence
  Synethic polynucleotide
atggcttctaggctgaccctgctgactctgctgctgctgctgctggccggcgacagggcttcttctaatcccaacgccacctctagcagcagccaggatcccgagagcctgcaggacaggggggaggggaaggtggccaccaccgtgatcagcaagatgctgttcgtggagcctatcctggaggtgtctagcctgcccaccaccaactctaccaccaacagcgccaccaagattactgctaacaccaccgacgagcccaccacccagcccactaccgagcctaccactcagcccaccattcagcccacccagcctactactcagctgcccactgacagccccacccagcctaccaccggcagcttttgccccggccccgtgaccctgtgcagcgacctggagagccattctaccgaggccgtgctgggcgacgccctggtggacttcagcctgaagctgtatcatgcctttagcgctatgaagaaggtggagactaacatggccttcagccccttctctatcgcctctctgctgacccaggtgctgctgggcgccggggagaacaccaagaccaatctggagagcatcctgagctaccccaaggatttcacttgcgtgcaccaggctctgaagggcttcaccaccaagggcgtgactagcgtgtctcagatcttccactctcccgacctggccattagggacactttcgtgaacgccagcaggactctgtacagcagcagccccagggtgctgagcaacaatagcgacgctaacctggagctgatcaacacctgggtggccaagaacaccaacaacaagatcagcaggctgctggacagcctgcccagcgataccaggctggtgctgctgaacgctatctacctgtctgctaagtggaaaactaccttcgatcctaagaaaaccaggatggagcccttccatttcaagaatagcgtgatcaaggtgcccatgatgaacagcaagaagtaccccgtggcccacttcatcgaccagaccctgaaggccaaggtgggccagctgcagctgagccacaacctgagcctggtgatcctggtgccccagaatctgaagcacaggctggaggacatggagcaggccctgagccccagcgtgttcaaggccatcatggagaagctggagatgtctaagttccagcccaccctgctgaccctgcccaggatcaaggtgaccactagccaggacatgctgagcattatggagaagctggagttcttcgacttctcttacgacctgaacctgtgcggcctgactgaggatcccgatctgcaggtgtctgctatgcagcaccagaccgtgctggagctgaccgagactggcgtggaggccgccgccgccagcgccatcagcgtggccaggaccctgctggtgttcgaggtgcagcagccctttctgtttgtgctgtgggatcagcagcacaagttccctgtgttcatggggagagtgtacgaccctagggcctga

SEQ ID NO: 14 - DNA, 32 bp, Artificial Sequence
  Synethic polynucleotide
tggacacaggacgctgtggtttctgagccagg

SEQ ID NO: 15 - DNA, 186 bp, Artificial Sequence
  Synethic polynucleotide
gggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 16 - DNA, 34 bp, Artificial Sequence
  Synethic polynucleotide
tgtttgctgcttgcaatgtttgcccattttaggg

SEQ ID NO: 17 - DNA, 351 bp, Artificial Sequence
  Synethic polynucleotide
gtcgacaggttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctctccccacccccaggagaggctcaggttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctctccccacccccaggagaggctgtcgac

SEQ ID NO: 18 - DNA, 252 bp, Artificial Sequence
  Synethic polynucleotide
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 19 - DNA, 227 bp, Artificial Sequence
  Synethic polynucleotide
agctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 20 - DNA, 3075 bp, Artificial Sequence
  Synethic polynucleotide
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgttcatgccttcttctttttcctacagctcctgggcaacgtgctggttgttgtgctgtctcatcattttggcaaacctgcaggccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 21 - DNA, 3134 bp, Artificial Sequence
  Synethic polynucleotide
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgttcatgccttcttctttttcctacagctcctgggcaacgtgctggttgttgtgctgtctcatcattttggcaaacctgcaggccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 22 - DNA, 3471 bp, Artificial Sequence
  Synethic polynucleotide
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgttcatgccttcttctttttcctacagctcctgggcaacgtgctggttgttgtgctgtctcatcattttggcaaacctgcaggccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 23 - DNA, 3900 bp, Artificial Sequence
  Synethic polynucleotide
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgttcatgccttcttctttttcctacagctcctgggcaacgtgctggttgttgtgctgtctcatcattttggcaaacctgcaggccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 24 - DNA, 3810 bp, Artificial Sequence
  Synethic polynucleotide
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggccagcaggctcactctgctgactctgctgctgctgctgctggctggggacagggccagcagcaaccccaatgccaccagcagcagcagccaggaccctgagagcctccaggacaggggggagggcaaggtggccaccactgtgatcagcaagatgctgtttgtggagcccatcctggaggtcagcagcctgcccaccaccaacagcaccaccaactctgccaccaagatcactgccaacactactgatgaacccactacccaacccaccactgagcccaccactcagcccactatccagcccacccagcccaccacccagctgcccactgacagccccacccagcccaccactggcagcttctgccctggccctgtgactctgtgcagtgatctggagagccacagcactgaggctgtgctgggggatgccctggtggacttcagcctgaagctgtaccacgccttcagtgctatgaagaaggtggagaccaatatggccttcagccccttcagtattgccagcctgctgactcaggtgctgctgggggctggggagaacactaagaccaacctggagagtatcctgagctaccccaaggacttcacttgtgtgcaccaggccctgaagggcttcactaccaagggggtcactagtgtgagccagatcttccacagccctgatctggccatcagggacacttttgtgaatgccagcaggaccctgtacagcagcagccccagggtgctgagcaacaacagtgatgccaacctggagctgatcaatacctgggtggccaagaataccaacaacaagatcagcaggctgctggacagcctgcccagtgataccaggctggtgctgctgaatgctatctacctgagtgccaagtggaagaccacctttgatcccaagaagaccaggatggagcccttccacttcaagaacagtgtgatcaaggtgcccatgatgaacagcaagaagtaccctgtggcccacttcattgatcagaccctgaaggccaaggtgggccagctgcagctgagccacaacctgagcctggtcatcctggtgccccagaacctgaagcacaggctggaggatatggagcaggccctgagccccagtgtgttcaaggctattatggagaagctcgaaatgagcaagttccagcccaccctgctgactctgcccaggatcaaggtcactaccagccaggatatgctgtctattatggagaaactggagttctttgacttcagctatgatctgaacctgtgtgggctgactgaggaccctgatctgcaagtgagtgccatgcagcaccagactgtgctggagctgactgagactggggtggaggctgctgctgctagtgccatcagtgtggccaggaccctgctggtctttgaggtgcagcagcccttcctgtttgtgctgtgggaccagcagcacaagttccctgtgttcatgggcagggtctatgatcccagggcctaacctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 25 - DNA, 3810 bp, Artificial Sequence
  Synethic polynucleotide
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggccagccgcctgaccctgctgaccctgctgctgctgctgctggccggcgaccgcgccagcagcaaccccaacgccaccagcagcagcagccaggaccccgagagcctgcaggaccgcggcgagggcaaggtggccaccaccgtgatcagcaagatgctgttcgtggagcccatcctggaggtgagcagcctgcccaccaccaacagcaccaccaacagcgccaccaagatcaccgccaacaccaccgacgagcccaccacccagcccaccaccgagcccaccacccagcccaccatccagcccacccagcccaccacccagctgcccaccgacagccccacccagcccaccaccggcagcttctgccccggccccgtgaccctgtgcagcgacctggagagccacagcaccgaggccgtgctgggcgacgccctggtggacttcagcctgaagctgtaccacgccttcagcgccatgaagaaggtggagaccaacatggccttcagccccttcagcatcgccagcctgctgacccaggtgctgctgggcgccggcgagaacaccaagaccaacctggagagcatcctgagctaccccaaggacttcacctgcgtgcaccaggccctgaagggcttcaccaccaagggcgtgaccagcgtgagccagatcttccacagccccgacctggccatccgcgacaccttcgtgaacgccagccgcaccctgtacagcagcagcccccgcgtgctgagcaacaacagcgacgccaacctggagctgatcaacacctgggtggccaagaacaccaacaacaagatcagccgcctgctggacagcctgcccagcgacacccgcctggtgctgctgaacgccatctacctgagcgccaagtggaagaccaccttcgaccccaagaagacccgcatggagcccttccacttcaagaacagcgtgatcaaggtgcccatgatgaacagcaagaagtaccccgtggcccacttcatcgaccagaccctgaaggccaaggtgggccagctgcagctgagccacaacctgagcctggtgatcctggtgccccagaacctgaagcaccgcctggaggacatggagcaggccctgagccccagcgtgttcaaggccatcatggagaagctggagatgagcaagttccagcccaccctgctgaccctgccccgcatcaaggtgaccaccagccaggacatgctgagcatcatggagaagctggagttcttcgacttcagctacgacctgaacctgtgcgggctgaccgaggaccccgacctgcaggtgagcgccatgcagcaccagaccgtgctggagctgaccgagaccggcgtggaggccgccgccgccagcgccatcagcgtggcccgcaccctgctggtgttcgaggtgcagcagcccttcctgttcgtgctgtgggaccagcagcacaagttccccgtgttcatgggccgcgtgtacgacccccgcgcctaacctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 26 - DNA, 3810 bp, Artificial Sequence
  Synethic polynucleotide
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggcttctaggctgaccctgctgactctgctgctgctgctgctggccggcgacagggcttcttctaatcccaacgccacctctagcagcagccaggatcccgagagcctgcaggacaggggggaggggaaggtggccaccaccgtgatcagcaagatgctgttcgtggagcctatcctggaggtgtctagcctgcccaccaccaactctaccaccaacagcgccaccaagattactgctaacaccaccgacgagcccaccacccagcccactaccgagcctaccactcagcccaccattcagcccacccagcctactactcagctgcccactgacagccccacccagcctaccaccggcagcttttgccccggccccgtgaccctgtgcagcgacctggagagccattctaccgaggccgtgctgggcgacgccctggtggacttcagcctgaagctgtatcatgcctttagcgctatgaagaaggtggagactaacatggccttcagccccttctctatcgcctctctgctgacccaggtgctgctgggcgccggggagaacaccaagaccaatctggagagcatcctgagctaccccaaggatttcacttgcgtgcaccaggctctgaagggcttcaccaccaagggcgtgactagcgtgtctcagatcttccactctcccgacctggccattagggacactttcgtgaacgccagcaggactctgtacagcagcagccccagggtgctgagcaacaatagcgacgctaacctggagctgatcaacacctgggtggccaagaacaccaacaacaagatcagcaggctgctggacagcctgcccagcgataccaggctggtgctgctgaacgctatctacctgtctgctaagtggaaaactaccttcgatcctaagaaaaccaggatggagcccttccatttcaagaatagcgtgatcaaggtgcccatgatgaacagcaagaagtaccccgtggcccacttcatcgaccagaccctgaaggccaaggtgggccagctgcagctgagccacaacctgagcctggtgatcctggtgccccagaatctgaagcacaggctggaggacatggagcaggccctgagccccagcgtgttcaaggccatcatggagaagctggagatgtctaagttccagcccaccctgctgaccctgcccaggatcaaggtgaccactagccaggacatgctgagcattatggagaagctggagttcttcgacttctcttacgacctgaacctgtgcggcctgactgaggatcccgatctgcaggtgtctgctatgcagcaccagaccgtgctggagctgaccgagactggcgtggaggccgccgccgccagcgccatcagcgtggccaggaccctgctggtgttcgaggtgcagcagccctttctgtttgtgctgtgggatcagcagcacaagttccctgtgttcatggggagagtgtacgaccctagggcctgagctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 27 - DNA, 4767 bp, Artificial Sequence
  Synethic polynucleotide
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggccagccgcctgaccctgctgaccctgctgctgctgctgctggccggcgaccgcgccagcagcaaccccaacgccaccagcagcagcagccaggaccccgagagcctgcaggaccgcggcgagggcaaggtggccaccaccgtgatcagcaagatgctgttcgtggagcccatcctggaggtgagcagcctgcccaccaccaacagcaccaccaacagcgccaccaagatcaccgccaacaccaccgacgagcccaccacccagcccaccaccgagcccaccacccagcccaccatccagcccacccagcccaccacccagctgcccaccgacagccccacccagcccaccaccggcagcttctgccccggccccgtgaccctgtgcagcgacctggagagccacagcaccgaggccgtgctgggcgacgccctggtggacttcagcctgaagctgtaccacgccttcagcgccatgaagaaggtggagaccaacatggccttcagccccttcagcatcgccagcctgctgacccaggtgctgctgggcgccggcgagaacaccaagaccaacctggagagcatcctgagctaccccaaggacttcacctgcgtgcaccaggccctgaagggcttcaccaccaagggcgtgaccagcgtgagccagatcttccacagccccgacctggccatccgcgacaccttcgtgaacgccagccgcaccctgtacagcagcagcccccgcgtgctgagcaacaacagcgacgccaacctggagctgatcaacacctgggtggccaagaacaccaacaacaagatcagccgcctgctggacagcctgcccagcgacacccgcctggtgctgctgaacgccatctacctgagcgccaagtggaagaccaccttcgaccccaagaagacccgcatggagcccttccacttcaagaacagcgtgatcaaggtgcccatgatgaacagcaagaagtaccccgtggcccacttcatcgaccagaccctgaaggccaaggtgggccagctgcagctgagccacaacctgagcctggtgatcctggtgccccagaacctgaagcaccgcctggaggacatggagcaggccctgagccccagcgtgttcaaggccatcatggagaagctggagatgagcaagttccagcccaccctgctgaccctgccccgcatcaaggtgaccaccagccaggacatgctgagcatcatggagaagctggagttcttcgacttcagctacgacctgaacctgtgcgggctgaccgaggaccccgacctgcaggtgagcgccatgcagcaccagaccgtgctggagctgaccgagaccggcgtggaggccgccgccgccagcgccatcagcgtggcccgcaccctgctggtgttcgaggtgcagcagcccttcctgttcgtgctgtgggaccagcagcacaagttccccgtgttcatgggccgcgtgtacgacccccgcgcctaacctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 28 - DNA, 4767 bp, Artificial Sequence
  Synethic polynucleotide
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggcctccagactgaccctcctcacactgctcctgctgctccttgctggagatagagccagctccaaccccaatgccacaagcagcagcagccaggaccctgagagcctgcaggacagaggagagggcaaggtggccacaacagtgatctctaaaatgctcttcgttgaacccatcctggaagtgtcctctctgcctaccacaaacagcaccacaaattcagccaccaagatcacagccaataccacagatgagcccacaacccaacctaccacagaaccaacaactcagcctacaatccagcctactcagccaaccacccagctgcctacagacagccccacacagcctacaacaggcagcttctgccctggacctgtgaccctgtgcagtgatctggaatctcactctacagaggctgtgctgggagatgccctggtggacttcagcctgaaactgtatcacgccttctctgccatgaaaaaggtggagacaaatatggcttttagccctttttctattgccagcctgctgacccaagtgctgctgggagctggagagaacacaaagaccaacttggaaagcatcctgagctaccctaaggacttcacctgtgtgcaccaggccctgaagggcttcacaaccaagggagtgacaagtgtttcccaaatcttccacagccctgatctggccatcagagacacctttgtgaatgcctccagaaccctgtacagcagctctcctagagtgctgagcaacaactctgatgccaacctggaactgatcaacacctgggtggccaaaaacacaaacaacaagatcagcagactgcttgatagcctcccttctgatactagactggtcctgctgaatgctatctacctgtctgctaagtggaagaccacttttgaccccaagaaaaccagaatggaacctttccactttaagaacagtgtgatcaaggtgcctatgatgaactccaagaaataccctgtggcccacttcattgaccagaccctgaaggccaaggtgggccagctgcaactgagccacaacctgagcctggtgattctggttcctcagaacctgaagcacagactggaagatatggagcaggccctgtctccatctgtgttcaaggccatcatggagaagctggaaatgagcaagttccagcccaccctgctgacactccccagaatcaaggtgaccaccagccaggatatgctgtctatcatggaaaagcttgagttctttgacttctcctatgacctgaacctctgtggcctgacagaagatccagacctccaggtgtctgccatgcagcaccagacagtgttggagctgacagaaacaggagtggaagctgcagctgcctctgccatctctgtggccagaacactgctggtgtttgaggtgcagcagcctttcctgtttgtgctgtgggaccaacagcacaagttccctgtgtttatgggcagagtgtatgaccctagagcctgacctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 29 - DNA, 4767 bp, Artificial Sequence
  Synethic polynucleotide
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggccagcagacttaccctgcttaccctgctcttgcttctgttggctggagacagagccagctccaatcctaatgctacctccagctccagtcaagatccagagagcctgcaggatagaggagagggcaaggtggccaccacagtcataagtaaaatgctctttgtggaacccatcctggaggtgagttctcttcctaccactaactctaccacaaactctgcaaccaagatcacagccaatactacagatgagcctacaactcaaccaactacagaacccacaactcaacctacaatccagcctactcaacccactacacaactgcccactgactcacctacccagcctaccacagggtcattttgtccagggccagttaccctgtgtagtgaccttgagagccatagtacagaggctgttctgggggatgctcttgtagattttagccttaaactgtaccatgctttcagtgctatgaaaaaagttgaaacaaacatggctttttccccttttagtattgcttctctccttactcaggttcttctgggagctggggagaacacaaaaactaatcttgaatctatcctcagctatcctaaagactttacttgtgtgcaccaagctcttaaaggatttacaactaagggagttactagtgtgagtcagatttttcacagtccagaccttgctatcagagatacatttgttaatgcttcaagaactctgtatagtagcagtcccagggtcttgtcaaataacagtgatgccaatcttgagctcataaatacctgggtggctaagaatacaaacaataaaatatcaaggcttctggactctctcccaagtgataccagacttgttctcctgaatgcaatatacctcagtgctaaatggaaaactacatttgatcccaaaaaaactagaatggagccatttcactttaaaaatagtgtaatcaaggtacctatgatgaacagcaaaaaatatcctgtagctcactttattgaccaaactctcaaagccaaagtggggcaactccagctgagtcacaacctgagtttggtaattctggtaccacaaaatctgaagcacagacttgaagatatggaacaagctctctctcccagtgtttttaaagccataatggaaaagcttgaaatgagtaaatttcaacccacactcctcaccctccctaggatcaaagttaccacatcccaggacatgctcagtataatggagaaacttgagttctttgattttagctatgatctgaatctttgtggcctgacagaggatccagatctccaggtatctgccatgcagcaccagactgtgctggagctgacagaaacaggtgtagaagctgcagcagcctcagctataagtgtggctagaacattgcttgtctttgaggtccaacagccttttttgtttgttctttgggaccaacagcataaatttcctgtctttatgggaagagtttatgacccaagagcttaacctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 30 - DNA, 4767 bp, Artificial Sequence
  Synethic polynucleotide
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggccagcagacttaccctgcttaccctgctcttgcttctgttggctggagacagagccagctccaatcctaatgctacctccagctccagtcaagatcccgagagcctgcaggatagaggagagggcaaggtggccaccacagtgatcagcaagatgctgtttgtggagcctatcctggaggtgagctctctgcctaccacaaatagcaccacaaattctgccaccaagatcacagccaataccactgatgaacccactacccagcccacaactgagcctacaacccaacctactatccaacctacccagcctaccactcaactgcccactgacagccctacccaacctacaactggctccttctgccctggccctgtgaccctgtgcagtgatctggagagccactctactgaggctgtgctgggagatgctctggtggacttcagcctgaagctgtaccacgccttctctgctatgaagaaggtggagaccaatatggctttcagccccttctctattgccagcctgctgacccaagtgctcctgggagctggagagaataccaagaccaatctggagtctatcctgagctaccccaaggatttcacctgtgtgcaccaagccctgaagggcttcaccacaaagggagtgacctctgtgagccagatcttccacagcccagatctggctatcagagatacctttgtgaatgccagcagaaccctgtacagctcttcccctagagtgctgagcaacaattctgatgctaatctggagctgatcaatacctgggtggccaagaataccaacaataagatcagcagactcctggactccctgccctctgataccagactggtgctcctgaatgctatctacctgtctgccaagtggaagaccacttttgaccccaagaagaccagaatggagcccttccacttcaagaattctgtgatcaaggtgcccatgatgaatagcaagaagtaccctgtggcccacttcattgatcagaccctgaaggctaaggtgggccaactccagctgagccacaatctcagcctggtgatcctggtgccccagaatctgaagcacagactggaggatatggagcaagccctgagcccctctgtgttcaaggctattatggaaaagcttgaaatgagcaagttccagcccacactcctgaccctgcccagaatcaaggtgaccactagccaagatatgctgtctattatggaaaaactggaattctttgacttcagctatgacctgaatctgtgtggcctgactgaagatcccgacctgcaagtgtctgccatgcagcaccaaactgtgctggagctcactgagacaggagtggaggctgcagctgcttctgccatctctgtggccagaaccctcctggtgtttgaggtgcaacagcctttcctgtttgtgctgtgggatcagcaacacaagttccctgtgttcatgggcagagtgtatgatcccagagcctaacctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 31 - DNA, 4280 bp, Artificial Sequence
  Synethic polynucleotide
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggcctccagactgaccctcctcacactgctcctgctgctccttgctggagatagagccagctccaaccccaatgccacaagcagcagcagccaggaccctgagagcctgcaggacagaggagagggcaaggtggccacaacagtgatctctaaaatgctcttcgttgaacccatcctggaagtgtcctctctgcctaccacaaacagcaccacaaattcagccaccaagatcacagccaataccacagatgagcccacaacccaacctaccacagaaccaacaactcagcctacaatccagcctactcagccaaccacccagctgcctacagacagccccacacagcctacaacaggcagcttctgccctggacctgtgaccctgtgcagtgatctggaatctcactctacagaggctgtgctgggagatgccctggtggacttcagcctgaaactgtatcacgccttctctgccatgaaaaaggtggagacaaatatggcttttagccctttttctattgccagcctgctgacccaagtgctgctgggagctggagagaacacaaagaccaacttggaaagcatcctgagctaccctaaggacttcacctgtgtgcaccaggccctgaagggcttcacaaccaagggagtgacaagtgtttcccaaatcttccacagccctgatctggccatcagagacacctttgtgaatgcctccagaaccctgtacagcagctctcctagagtgctgagcaacaactctgatgccaacctggaactgatcaacacctgggtggccaaaaacacaaacaacaagatcagcagactgcttgatagcctcccttctgatactagactggtcctgctgaatgctatctacctgtctgctaagtggaagaccacttttgaccccaagaaaaccagaatggaacctttccactttaagaacagtgtgatcaaggtgcctatgatgaactccaagaaataccctgtggcccacttcattgaccagaccctgaaggccaaggtgggccagctgcaactgagccacaacctgagcctggtgattctggttcctcagaacctgaagcacagactggaagatatggagcaggccctgtctccatctgtgttcaaggccatcatggagaagctggaaatgagcaagttccagcccaccctgctgacactccccagaatcaaggtgaccaccagccaggatatgctgtctatcatggaaaagcttgagttctttgacttctcctatgacctgaacctctgtggcctgacagaagatccagacctccaggtgtctgccatgcagcaccagacagtgttggagctgacagaaacaggagtggaagctgcagctgcctctgccatctctgtggccagaacactgctggtgtttgaggtgcagcagcctttcctgtttgtgctgtgggaccaacagcacaagttccctgtgtttatgggcagagtgtatgaccctagagcctgacctcgagcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcctgggcctagggctgtgccagctgcctcgtcccgtcaccttctggcttcttctctccctccatatcttagctgttttcctcatgagaatgttccaaattcgaaatttctatttaaccattatatatttacttgtttgctattatctctgcccccagtagattgttagctccagaagagaaaggatcatgtcttttgcttatctagatatgcccatctgcctggtacaatctctggcacatgttacaggcaacaactacttgtggaattggtgaatgcatgaatagaagaatgagtgaatgaatgaatagacaaaaggcagaaatccagcctcaaagagcttacagtctggtaagaggaataaaatgtctgcaaatagccacaggacaggtcaaaggaaggaggggctatttccagctgagggcaccccatcaggaaagcaccccagacttcctacaactactagacacatctcgatgcttttcacttctctatcaatgggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 32 - DNA, 4280 bp, Artificial Sequence
  Synethic polynucleotide
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggccagcagacttaccctgcttaccctgctcttgcttctgttggctggagacagagccagctccaatcctaatgctacctccagctccagtcaagatccagagagcctgcaggatagaggagagggcaaggtggccaccacagtcataagtaaaatgctctttgtggaacccatcctggaggtgagttctcttcctaccactaactctaccacaaactctgcaaccaagatcacagccaatactacagatgagcctacaactcaaccaactacagaacccacaactcaacctacaatccagcctactcaacccactacacaactgcccactgactcacctacccagcctaccacagggtcattttgtccagggccagttaccctgtgtagtgaccttgagagccatagtacagaggctgttctgggggatgctcttgtagattttagccttaaactgtaccatgctttcagtgctatgaaaaaagttgaaacaaacatggctttttccccttttagtattgcttctctccttactcaggttcttctgggagctggggagaacacaaaaactaatcttgaatctatcctcagctatcctaaagactttacttgtgtgcaccaagctcttaaaggatttacaactaagggagttactagtgtgagtcagatttttcacagtccagaccttgctatcagagatacatttgttaatgcttcaagaactctgtatagtagcagtcccagggtcttgtcaaataacagtgatgccaatcttgagctcataaatacctgggtggctaagaatacaaacaataaaatatcaaggcttctggactctctcccaagtgataccagacttgttctcctgaatgcaatatacctcagtgctaaatggaaaactacatttgatcccaaaaaaactagaatggagccatttcactttaaaaatagtgtaatcaaggtacctatgatgaacagcaaaaaatatcctgtagctcactttattgaccaaactctcaaagccaaagtggggcaactccagctgagtcacaacctgagtttggtaattctggtaccacaaaatctgaagcacagacttgaagatatggaacaagctctctctcccagtgtttttaaagccataatggaaaagcttgaaatgagtaaatttcaacccacactcctcaccctccctaggatcaaagttaccacatcccaggacatgctcagtataatggagaaacttgagttctttgattttagctatgatctgaatctttgtggcctgacagaggatccagatctccaggtatctgccatgcagcaccagactgtgctggagctgacagaaacaggtgtagaagctgcagcagcctcagctataagtgtggctagaacattgcttgtctttgaggtccaacagccttttttgtttgttctttgggaccaacagcataaatttcctgtctttatgggaagagtttatgacccaagagcttaacctcgagcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcctgggcctagggctgtgccagctgcctcgtcccgtcaccttctggcttcttctctccctccatatcttagctgttttcctcatgagaatgttccaaattcgaaatttctatttaaccattatatatttacttgtttgctattatctctgcccccagtagattgttagctccagaagagaaaggatcatgtcttttgcttatctagatatgcccatctgcctggtacaatctctggcacatgttacaggcaacaactacttgtggaattggtgaatgcatgaatagaagaatgagtgaatgaatgaatagacaaaaggcagaaatccagcctcaaagagcttacagtctggtaagaggaataaaatgtctgcaaatagccacaggacaggtcaaaggaaggaggggctatttccagctgagggcaccccatcaggaaagcaccccagacttcctacaactactagacacatctcgatgcttttcacttctctatcaatgggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 33 - DNA, 4280 bp, Artificial Sequence
  Synethic polynucleotide
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggccagcagacttaccctgcttaccctgctcttgcttctgttggctggagacagagccagctccaatcctaatgctacctccagctccagtcaagatcccgagagcctgcaggatagaggagagggcaaggtggccaccacagtgatcagcaagatgctgtttgtggagcctatcctggaggtgagctctctgcctaccacaaatagcaccacaaattctgccaccaagatcacagccaataccactgatgaacccactacccagcccacaactgagcctacaacccaacctactatccaacctacccagcctaccactcaactgcccactgacagccctacccaacctacaactggctccttctgccctggccctgtgaccctgtgcagtgatctggagagccactctactgaggctgtgctgggagatgctctggtggacttcagcctgaagctgtaccacgccttctctgctatgaagaaggtggagaccaatatggctttcagccccttctctattgccagcctgctgacccaagtgctcctgggagctggagagaataccaagaccaatctggagtctatcctgagctaccccaaggatttcacctgtgtgcaccaagccctgaagggcttcaccacaaagggagtgacctctgtgagccagatcttccacagcccagatctggctatcagagatacctttgtgaatgccagcagaaccctgtacagctcttcccctagagtgctgagcaacaattctgatgctaatctggagctgatcaatacctgggtggccaagaataccaacaataagatcagcagactcctggactccctgccctctgataccagactggtgctcctgaatgctatctacctgtctgccaagtggaagaccacttttgaccccaagaagaccagaatggagcccttccacttcaagaattctgtgatcaaggtgcccatgatgaatagcaagaagtaccctgtggcccacttcattgatcagaccctgaaggctaaggtgggccaactccagctgagccacaatctcagcctggtgatcctggtgccccagaatctgaagcacagactggaggatatggagcaagccctgagcccctctgtgttcaaggctattatggaaaagcttgaaatgagcaagttccagcccacactcctgaccctgcccagaatcaaggtgaccactagccaagatatgctgtctattatggaaaaactggaattctttgacttcagctatgacctgaatctgtgtggcctgactgaagatcccgacctgcaagtgtctgccatgcagcaccaaactgtgctggagctcactgagacaggagtggaggctgcagctgcttctgccatctctgtggccagaaccctcctggtgtttgaggtgcaacagcctttcctgtttgtgctgtgggatcagcaacacaagttccctgtgttcatgggcagagtgtatgatcccagagcctaacctcgagcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcctgggcctagggctgtgccagctgcctcgtcccgtcaccttctggcttcttctctccctccatatcttagctgttttcctcatgagaatgttccaaattcgaaatttctatttaaccattatatatttacttgtttgctattatctctgcccccagtagattgttagctccagaagagaaaggatcatgtcttttgcttatctagatatgcccatctgcctggtacaatctctggcacatgttacaggcaacaactacttgtggaattggtgaatgcatgaatagaagaatgagtgaatgaatgaatagacaaaaggcagaaatccagcctcaaagagcttacagtctggtaagaggaataaaatgtctgcaaatagccacaggacaggtcaaaggaaggaggggctatttccagctgagggcaccccatcaggaaagcaccccagacttcctacaactactagacacatctcgatgcttttcacttctctatcaatgggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 34 - DNA, 4767 bp, Artificial Sequence
  Synethic polynucleotide
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 35 - DNA, 4385 bp, Artificial Sequence
  Synethic polynucleotide
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagtttttagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 36 - DNA, 3765 bp, Artificial Sequence; identical to SEQ ID NO: 8
  Synethic polynucleotide

SEQ ID NO: 37 - protein, 736 aa, Adeno-Associated Virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 38 - protein, 736 aa, Adeno-Associated Virus Rh.8
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 39 - protein, 738 aa, Adeno-Associated Virus Rh.10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 40 - protein, 738 aa, Adeno-Associated Virus Rh.74
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 41 - protein, 736 aa, Adeno-Associated Virus 1
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 42 - protein, 735 aa, Adeno-Associated Virus 2 [sequence omitted]

SEQ ID NO: 43 - protein, 736 aa, Adeno-Associated Virus 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 44 - protein, 736 aa, Adeno-Associated Virus 3B; identical to SEQ ID NO: 41

SEQ ID NO: 45 - protein, 734 aa, Adeno-Associated Virus 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 46 - protein, 724 aa, Adeno-Associated Virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 47 - protein, 736 aa, Adeno-Associated Virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 48 - protein, 737 aa, Adeno-Associated Virus 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 49 - protein, 738 aa, Adeno-Associated Virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 50 - protein, 738 aa, Adeno-Associated Virus 10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 51 - protein, 733 aa, Adeno-Associated Virus 11
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 52 - protein, 742 aa, Adeno-Associated Virus 12
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 53 - protein, 733 aa, Adeno-Associated Virus 13
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAKTAPGKKRPVEQSPAEPDSSSGIGKSGQQPARKRLNFGQTGDTESVPDPQPLGQPPAAPSGVGSTTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYHLNGRDSLVNPGPAMASHKDDKEKFFPMHGTLIFGKEGTNANNADLENVMITDEEEIRTTNPVATEQYGTVSNNLQNSNAGPTTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 54 - DNA, 130 bp, Adeno-Associated Virus
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 55 - DNA, 130 bp, Adeno-Associated Virus
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 56 - protein, 742 aa, Artificial Sequence
  Synthetic polypeptide
MAADGYLPDWLEDNLSEGIREWWALKPGAPQPKANQQHQDNARGLVLPGYKYLGPGNGLDKGEPVNEADAAALEHDKAYDQQLKSGDNPYLKYNHADAEFQERLQEDTSFGGNLGRAVFQAKKRILEPLGLVEEGVKTAPGKKRPLEKTPNRPTNPDSGKAPAKKKQKDGETADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSLPPFPNDVFMVPQYGYCGVVTGENQNQTDRNAFYCLEYFPSQMLRTGNNFEISYQFEKVPFHSMYAHSQSLDRMMNPLLDQYLWHLQSTTTGNSLNQGTAITTYGKITTGDFAYYRKNWLPGAGIKQQKFSKNASQNYKIPASGGDALLKYDTHTTLNGRWSNMAPGPPMATAGAGDSDFSNSQLIFAGPNQSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHEPRAIGSRFLTHHL

SEQ ID NO: 57 - DNA, 2954 bp, Artificial Sequence
  Synthetic polynucleotide
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 58 - DNA, 3853 bp, Artificial Sequence
  Synthetic polynucleotide
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgacctcgagcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcctgggcctagggctgtgccagctgcctcgtcccgtcaccttctggcttcttctctccctccatatcttagctgttttcctcatgagaatgttccaaattcgaaatttctatttaaccattatatatttacttgtttgctattatctctgcccccagtagattgttagctccagaagagaaaggatcatgtcttttgcttatctagatatgcccatctgcctggtacaatctctggcacatgttacaggcaacaactacttgtggaattggtgaatgcatgaatagaagaatgagtgaatgaatgaatagacaaaaggcagaaatccagcctcaaagagcttacagtctggtaagaggaataaaatgtctgcaaatagccacaggacaggtcaaaggaaggaggggctatttccagctgagggcaccccatcaggaaagcaccccagacttcctacaactactagacacatctcgatgcttttcacttctctatcaatgggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 59 - DNA, 1508 bp, Artificial Sequence
  Synthetic polynucleotide
atggccagcaggctcactctgctgactctgctgctgctgctgctggctggggacagggccagcagcaaccccaatgccaccagcagcagcagccaggaccctgagagcctccaggacaggggggagggcaaggtggccaccactgtgatcagcaagatgctgtttgtggagcccatcctggaggtcagcagcctgcccaccaccaacagcaccaccaactctgccaccaagatcactgccaacactactgatgaacccactacccaacccaccactgagcccaccactcagcccactatccagcccacccagcccaccacccagctgcccactgacagccccacccagcccaccactggcagcttctgccctggccctgtgactctgtgcagtgatctggagagccacagcactgaggctgtgctgggggatgccctggtggacttcagcctgaagctgtaccacgccttcagtgctatgaagaaggtggagaccaatatggccttcagccccttcagtattgccagcctgctgactcaggtgctgctgggggctggggagaacactaagaccaacctggagagtatcctgagctaccccaaggacttcacttgtgtgcaccaggccctgaagggcttcactaccaagggggtcactagtgtgagccagatcttccacagccctgatctggccatcagggacacttttgtgaatgccagcaggaccctgtacagcagcagccccagggtgctgagcaacaacagtgatgccaacctggagctgatcaatacctgggtggccaagaataccaacaacaagatcagcaggctgctggacagcctgcccagtgataccaggctggtgctgctgaatgctatctacctgagtgccaagtggaagaccacctttgatcccaagaagaccaggatggagcccttccacttcaagaacagtgtgatcaaggtgcccatgatgaacagcaagaagtaccctgtggcccacttcattgatcagaccctgaaggccaaggtgggccagctgcagctgagccacaacctgagcctggtcatcctggtgccccagaacctgaagcacaggctggaggatatggagcaggccctgagccccagtgtgttcaaggctattatggagaagctcgaaatgagcaagttccagcccaccctgctgactctgcccaggatcaaggtcactaccagccaggatatgctgtctattatggagaaactggagttctttgacttcagctatgatctgaacctgtgtgggctgactgaggaccctgatctgcaagtgagtgccatgcagcaccagactgtgctggagctgactgagactggggtggaggctgctgctgctagtgccatcagtgtggccaggaccctgctggtctttgaggtgcagcagcccttcctgtttgtgctgtgggaccagcagcacaagttccctgtgttcatgggcagggtctatgatcccagggcctaacctcg

SEQ ID NO: 60 - DNA, 1508 bp, Artificial Sequence
  Synthetic polynucleotide
atggccagccgcctgaccctgctgaccctgctgctgctgctgctggccggcgaccgcgccagcagcaaccccaacgccaccagcagcagcagccaggaccccgagagcctgcaggaccgcggcgagggcaaggtggccaccaccgtgatcagcaagatgctgttcgtggagcccatcctggaggtgagcagcctgcccaccaccaacagcaccaccaacagcgccaccaagatcaccgccaacaccaccgacgagcccaccacccagcccaccaccgagcccaccacccagcccaccatccagcccacccagcccaccacccagctgcccaccgacagccccacccagcccaccaccggcagcttctgccccggccccgtgaccctgtgcagcgacctggagagccacagcaccgaggccgtgctgggcgacgccctggtggacttcagcctgaagctgtaccacgccttcagcgccatgaagaaggtggagaccaacatggccttcagccccttcagcatcgccagcctgctgacccaggtgctgctgggcgccggcgagaacaccaagaccaacctggagagcatcctgagctaccccaaggacttcacctgcgtgcaccaggccctgaagggcttcaccaccaagggcgtgaccagcgtgagccagatcttccacagccccgacctggccatccgcgacaccttcgtgaacgccagccgcaccctgtacagcagcagcccccgcgtgctgagcaacaacagcgacgccaacctggagctgatcaacacctgggtggccaagaacaccaacaacaagatcagccgcctgctggacagcctgcccagcgacacccgcctggtgctgctgaacgccatctacctgagcgccaagtggaagaccaccttcgaccccaagaagacccgcatggagcccttccacttcaagaacagcgtgatcaaggtgcccatgatgaacagcaagaagtaccccgtggcccacttcatcgaccagaccctgaaggccaaggtgggccagctgcagctgagccacaacctgagcctggtgatcctggtgccccagaacctgaagcaccgcctggaggacatggagcaggccctgagccccagcgtgttcaaggccatcatggagaagctggagatgagcaagttccagcccaccctgctgaccctgccccgcatcaaggtgaccaccagccaggacatgctgagcatcatggagaagctggagttcttcgacttcagctacgacctgaacctgtgcgggctgaccgaggaccccgacctgcaggtgagcgccatgcagcaccagaccgtgctggagctgaccgagaccggcgtggaggccgccgccgccagcgccatcagcgtggcccgcaccctgctggtgttcgaggtgcagcagcccttcctgttcgtgctgtgggaccagcagcacaagttccccgtgttcatgggccgcgtgtacgacccccgcgcctaacctcg

SEQ ID NO: 61 - DNA, 196 bp, Artificial Sequence
  Synthetic polynucleotide
gtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccacc

SEQ ID NO: 62 - DNA, 317 bp, Artificial Sequence
  Synthetic polynucleotide
gtaagtatgcctttcactgcgaggggttctggagaggcttagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgttcatgccttcttctttttcctacagctcctgggcaacgtgctggttgttgtgctgtctcatcattttggcaaacctgcaggccacc

SEQ ID NO: 63 - DNA, 376 bp, Artificial Sequence
  Synthetic polynucleotide
gtaagtatgcctttcactgcgaggggttctggagaggcttctgagctccccatggcccaggcaggcagcaggtctggggcaggaggggggttgtggagtagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgttcatgccttcttctttttcctacagctcctgggcaacgtgctggttgttgtgctgtctcatcattttggcaaacctgcaggccacc

SEQ ID NO: 64 - DNA, 435 bp, Artificial Sequence
  Synthetic polynucleotide
gtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggca

SEQ ID NO: 65 - DNA, 864 bp, Artificial Sequence
  Synthetic polynucleotide
gtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactct

SEQ ID NO: 66 - DNA, 1439 bp, Artificial Sequence
  Synthetic polynucleotide
gtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagtttt

SEQ ID NO: 67 - DNA, 1821 bp, Artificial Sequence
  Synthetic polynucleotide
gtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgc

SEQ ID NO: 68 - DNA, 130 bp, Artificial Sequence
  Synthetic polynucleotide
gtaagtatgcctttcactgcgagaggttctggagaggcttctgagctccccatggcccaggcaggcagcaggtctggggcaggaggggggttgtggagtgggtatccgcctgctgaggtgcagggcagat

SEQ ID NO: 69 - DNA, 98 bp, Artificial Sequence
  Synthetic polynucleotide
gtaagtatgcctttcactgcgaggggttctggagaggcttctgagctccccatggcccaggcaggcagcaggtctggggcaggaggggggttgtggag

SEQ ID NO: 70 - DNA, 123 bp, Artificial Sequence
  Synthetic polynucleotide
tagcagctacaatccagctaccattctgcttttattttatggttgggataaggctggattattctgagtccaagctaggcccttttgctaatcatgttcatacctcttatcttcctcccacag

SEQ ID NO: 71 - DNA, 53 bp, Artificial Sequence
  Synthetic polynucleotide
ctcctgggcaacgtgctggtctgtgtgctggcccatcactttggcaaagaatt

SEQ ID NO: 72 - DNA, 131 bp, Artificial Sequence
  Synthetic polynucleotide
catcatgtgccttgactcggggcctggcccccccatctctgtcttgcaggacaattgccgtcttctgtctcgtggggcatcctcctgctggcaggcctgtgctgcctggtccctgtctccctggctgagga